(19) United States
(12) Patent Application Publication
(10) Pub. No.: US 2019/0352717 A1
(43) Pub. Date: Nov. 21, 2019

(54) TARGETED NON-INVASIVE PRENATAL TESTING

(71) Applicant: 10X GENOMICS, INC., Pleasanton, CA (US)

(72) Inventor: Michael Schnall-Levin, San Francisco, CA (US)

(21) Appl. No.: 16/415,617

(22) Filed: May 17, 2019

Related U.S. Application Data
(60) Provisional application No. 62/673,302, filed on May 18, 2018.

(51) Int. Cl. C12Q 1/6883; C12Q 1/6806; C12Q 1/6837; C12Q 1/6874

(52) U.S. Cl. CPC C12Q 1/6883; C12Q 1/6806; C12Q 2600/156; C12Q 1/6874; C12Q 2600/118; C12Q 1/6837

(57) ABSTRACT

The present disclosure relates to methods, compositions and systems for targeted haplotype phasing, SNP identification, and copy number variation assays. Included within this disclosure are methods and systems for combining oligonucleotide barcodes with nucleic acid samples in multiple separate partitions, as well as methods of processing, sequencing and analyzing barcoded samples.

### CROSS-REFERENCE

This application claims the benefit of U.S. Provisional Patent Application No. 62/673,302, filed May 18, 2018, which application is entirely incorporated herein by reference.

### BACKGROUND

Non-invasive prenatal testing (NIPT) can be used to identify abnormalities in fetal DNA, such as fetal DNA derived from a maternal cell free DNA (cfDNA) sample. A fundamental understanding of a particular fetal genome may require more than simply identifying the presence or absence of certain genetic variations such as mutations. In many circumstances, it is also important to determine whether certain genetic variations appear on the same or different chromosomes (also known as phasing), or whether a particular variant was maternally or paternally inherited. Information about patterns of genetic variations, such as haplotypes may also be important, in addition to information about the number of copies of genes.

The term “haplotype” refers to sets of DNA sequence variants (alleles) that are inherited together in contiguous blocks. In general, the human genome contains two copies of each gene—a maternal copy and a paternal copy. For a pair of genes each having two possible alleles, for example gene alleles “A” and “a”, and gene alleles “B” and “b”, the genome of a given individual will include one of two haplotypes, “AB/ab”, where the A and B alleles reside on the same chromosome (the “cis” configuration), or “Ab/aB, where the A and B alleles reside on different chromosomes (the “trans” configuration). Phasing methods or assays can be used to determine whether a specified set of alleles reside on the same or different chromosomes. In some cases, several linked alleles that define a haplotype may correlate with, or be associated with, a particular disease phenotype; in such cases, a haplotype, rather than any one particular genetic variant, may be the most determinative factor as to whether a patient will display the disease.

Gene copy number may also play a role in some disease phenotypes. Most genes are normally present in two copies, however, amplified genes are genes that are present in more than two functional copies. In some instances, genes may undergo a loss of one or more functional copies. A loss or gain in gene copy number can lead to the production of abnormal levels of mRNA and protein expression, potentially leading to a cancerous state or other disorder. Cancer and other genetic disorders are often correlated with abnormal (increased or decreased) chromosome numbers (“aneuploidy.”) Cytogenetic techniques such as fluorescence in situ hybridization or comparative genomic hybridization can be used to detect the presence of abnormal gene or chromosome copy numbers, but improved methods of detecting genetic phasing information, haplotypes, or copy number variations are needed in the art.

### SUMMARY

Detection of paternally inherited fetal single nucleotide polymorphisms (SNPs) can be determined based on SNPs present in a maternal cell free DNA (cfDNA) sample that are absent from the maternal genome. Alternatively, methods that utilize haplotyping information to increase the ability to call mutations, such as relative haplotype dosing analysis (RHDO) can be used to determine the maternally derived half of the fetal genome. This technique can decipher genomic regions for which the father is homozygous and the mother is heterozygous based on comparing the relative concentrations of such haplotypes in a maternal cfDNA sample. Specifically, RHDO can be performed using sequential probability ratio tests (SPRT)-based classification.

The present disclosure provides methods and systems that may be useful in providing significant advances in the characterization of genetic material. In some cases, genetic material from a fetus may be characterized, specifically determining the source of fetal genomic variation as maternal or paternal in source. These methods and systems can be useful in providing genetic characterizations that may be substantially difficult using generally available technologies, including, for example, haplotype phasing, identifying structural variations, e.g., deletions, duplications, copy-number variants, insertions, inversions, translocations, long tandem repeats (LTRs), short tandem repeats (STRs), and a variety of other useful characterizations. Furthermore, the present disclosure provides methods and systems for generation of a phased targeted library of parental DNA from, e.g., a maternal cfDNA sample.

Disclosed herein in some embodiments, are methods for nucleic acid analysis, comprising: (a) generating a plurality of barcoded parental nucleic acid molecules in a plurality of partitions using (i) a plurality of parental nucleic acid molecules derived from a parental biological sample, and (ii) a plurality of nucleic acid barcode molecules; (b) enriching the plurality of barcoded parental nucleic acid molecules or derivatives thereof for target nucleic acid molecules comprising one or more target regions to generate an enriched set of barcoded parental nucleic acid molecules; (c) using the enriched set of barcoded parental nucleic acid molecules or derivatives thereof to generate parental nucleic acid sequence information comprising one or more nucleic acid sequences of the plurality of parental nucleic acid molecules; (d) processing the parental nucleic acid sequence information to identify one or more maternal or paternal haplotype blocks from the parental biological sample; and (e) processing cell-free nucleic acid sequence information derived from a maternal cell-free biological sample against the one or more maternal or paternal haplotype blocks, to identify one or more genomic variations in one or more fetal nucleic acid sequences of the maternal cell-free biological sample. In some embodiments, the processing in (e) comprises performing a relative haplotype dosing analysis. In some embodiments, performing the relative haplotype dosing analysis comprises performing a sequential probability ratio test of allelic imbalance in the cell-free nucleic acid sequence information derived from a maternal cell-free biological sample.

In some embodiments, the aforementioned methods disclosed herein further comprise, prior to (a), generating a plurality of partitions comprising (i) the plurality of parental nucleic acid molecules, and (ii) the plurality of nucleic acid barcode molecules. In some embodiments, in (c), the parental nucleic acid sequence information is generated by sequencing the enriched set of barcoded parental nucleic acid molecules or derivatives thereof. In some embodiments, prior to (b), the plurality of barcoded parental nucleic acid molecules are removed or released from the plurality of partitions. In some embodiments, the enriching of (b) is performed using nucleic acid capture of the one or more target regions in the plurality of barcoded parental nucleic acid molecules. In some embodiments, the nucleic acid capture is exome capture. In some embodiments, the enriching of (b) is performed by nucleic acid amplification of the one or more target regions in the plurality of barcoded parental nucleic acid molecules.

In some embodiments, the aforementioned methods disclosed herein further comprise obtaining, from a subject having a fetus, a maternal biological sample, and deriving from the maternal biological sample (i) the plurality of parental nucleic acid molecules, and (ii) the maternal cell-free biological sample comprising one or more fetal nucleic acid molecules of the fetus. In some embodiments, the maternal biological sample is whole blood. In some embodiments, the maternal biological sample is a buffy coat sample from the whole blood. In some embodiments, the maternal cell-free biological sample is a plasma sample from the whole blood.

In some embodiments, the afore mentioned methods disclosed herein further comprise sequencing the one or more fetal nucleic acid molecules of the maternal cell-free biological sample to generate the cell-free nucleic acid sequence information. In some embodiments, in (a), the plurality of parental nucleic acid molecules is derived from a maternal biological sample, and wherein the parental nucleic acid sequence information in (d) comprises one or more haplotype blocks derived from the maternal biological sample.

In some embodiments, the aforementioned methods disclosed herein further comprise generating paternal nucleic acid sequence information from a plurality of nucleic acid molecules derived from a paternal biological sample, and processing the paternal nucleic acid sequence information to identify one or more maternal or paternal haplotype blocks from the paternal biological sample. In some embodiments, a given partition of the plurality of partitions comprises a parental nucleic acid molecule from the plurality of parental nucleic acid molecules, wherein the parental nucleic acid molecule has a length longer than 10 kilobases. In some embodiments, the parental nucleic acid molecule has a length longer than 100 kilobases. In some embodiments, the plurality of partitions further comprise a plurality of beads, wherein a given bead of the plurality of beads comprises a plurality of nucleic acid barcode molecules attached thereto, and wherein a given partition of the plurality of partitions further comprises a single bead. In some embodiments, the plurality of partitions is a plurality of droplets. In some embodiments, the plurality of partitions is a plurality of wells.

Also disclosed herein, in some embodiments, are methods for nucleic acid analysis, comprising: (a) providing a plurality of parental nucleic acid molecules derived from a parental biological sample and a plurality of beads, wherein a given bead of the plurality of beads comprises a plurality of nucleic acid barcode molecules attached thereto, and wherein the plurality of nucleic acid barcode molecules comprise a sequence complementary to one or more target sequences of the plurality of parental nucleic acid molecules; (b) generating a plurality of partitions, wherein a given partition of the plurality of partitions comprises (i) a parental nucleic acid molecule from the plurality of parental nucleic acid molecules, and (ii) a single bead from the plurality of beads; (c) in the plurality of partitions, synthesizing a plurality of barcoded, targeted parental nucleic acid molecules using (i) parental nucleic acid molecules from the plurality of parental nucleic acid molecules, and (ii) nucleic acid barcode molecules from the plurality of nucleic acid barcode molecules, wherein the barcoded, targeted parental nucleic acid molecules comprise the one or more target sequences; (d) using the barcoded, targeted parental nucleic acid molecules or derivatives thereof to generate parental nucleic acid sequence information comprising one or more nucleic acid sequences of the plurality of parental nucleic acid molecules; (e) processing the parental nucleic acid sequence information to identify one or more maternal or paternal haplotype blocks from the parental biological sample; and (f) processing cell-free nucleic acid sequence information derived from a maternal cell-free biological sample against the one or more maternal or paternal haplotype blocks, to identify one or more genomic variations in one or more fetal nucleic acid sequences of the cell-free nucleic acid sequence information. In some embodiments, the processing in (f) comprises performing a relative haplotype dosing analysis. In some embodiments, performing the relative haplotype dosing analysis comprises performing a sequential probability ratio test of allelic imbalance in the cell-free nucleic acid sequence information derived from a maternal cell-free biological sample. In some embodiments, in (d), the parental nucleic acid sequence information is generated by sequencing the barcoded, targeted parental nucleic acid molecules or derivatives thereof.

In some embodiments, the aforementioned methods disclosed herein further comprise obtaining, from a subject having a fetus, a maternal biological sample, and deriving from the maternal biological sample (i) the plurality of parental nucleic acid molecules, and (ii) the maternal cell-free biological sample comprising one or more fetal nucleic acid molecules of the fetus. In some embodiments, the maternal biological sample is whole blood. In some embodiments, the maternal biological sample is a buffy coat sample from the whole blood. In some embodiments, the maternal cell-free biological sample is a plasma sample from the whole blood.

In some embodiments, the methods described herein further comprise sequencing the one or more fetal nucleic acid molecules of the maternal cell-free biological sample to generate the cell-free nucleic acid sequence information. In some embodiments, in (a), the plurality of parental nucleic acid molecules is derived from a maternal biological sample, and wherein the parental nucleic acid sequence information in (e) comprises one or more haplotype blocks derived from the maternal biological sample.

In some embodiments, the aforementioned methods disclosed herein further comprise generating paternal nucleic acid sequence information from a plurality of nucleic acid molecules derived from a paternal biological sample, and processing the paternal nucleic acid sequence information to identify one or more maternal or paternal haplotype blocks from the parental biological sample. In some embodiments, the parental nucleic acid molecule from the plurality of parental nucleic acid molecules has a length longer than 1 kilobase (kb). In some embodiments, the parental nucleic acid molecule from the plurality of parental nucleic acid molecules has a length longer than 10 kb. In some embodiments, the plurality of partitions is a plurality of droplets. In some embodiments, the plurality of partitions is a plurality of wells.

Disclosed herein, in some embodiments, are methods for nucleic acid analysis, comprising: (a) generating a plurality of partitions comprising (i) a plurality of parental nucleic acid molecules derived from a parental biological sample, (ii) a plurality of nucleic acid barcode molecules, and (iii) a plurality of oligonucleotide primers, wherein the plurality of oligonucleotide primers is capable of amplifying one or more target sequences of the plurality of parental nucleic acid molecules; (b) in the plurality of partitions, generating a plurality of amplified parental nucleic acid molecules using (i) nucleic acid molecules from the plurality of parental nucleic acid molecules, and (ii) oligonucleotide primers from the plurality of oligonucleotide primers; (c) in the plurality of partitions, generating a plurality of barcoded, amplified parental nucleic acid molecules using (i) amplified parental nucleic acid molecules from the plurality of amplified parental nucleic acid molecules and (ii) nucleic acid barcode molecules from the plurality of nucleic acid barcode molecules; (d) sequencing the plurality of barcoded, amplified parental nucleic acid molecules or derivatives thereof to generate parental nucleic acid sequence information comprising one or more nucleic acid sequences of the plurality of parental nucleic acid molecules; (e) processing the parental nucleic acid sequence information to identify one or more maternal or paternal haplotype blocks from the parental biological sample; and (f) processing cell-free nucleic acid sequence information derived from a maternal cell-free biological sample against the one or more maternal or paternal haplotype blocks, to identify one or more genomic variations in one or more fetal nucleic acid sequences of the cell-free nucleic acid sequence information. In some embodiments, the processing in (f) comprises performing a relative haplotype dosing analysis. In some embodiments, the plurality of partitions is a plurality of droplets. In some embodiments, the plurality of partitions is a plurality of wells.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 provides a schematic illustration of identification and analysis of phased variants using conventional processes versus example processes and systems described herein.

FIG. 2 provides a schematic illustration of the identification and analysis of structural variations using conventional processes versus example processes and systems described herein.

FIG. 3 illustrates an example workflow for performing an assay to detect copy number or haplotype using methods and compositions disclosed herein.

FIG. 4 provides a schematic illustration of an example process for combining a nucleic acid sample with beads and partitioning the nucleic acids and beads into discrete droplets.

FIG. 5 provides a schematic illustration of an example process for barcoding and amplification of chromosomal nucleic acid fragments.

FIG. 6 provides a schematic illustration of an example use of barcoding of chromosomal nucleic acid fragments in attributing sequence data to individual chromosomes.

FIG. 7 provides a schematic illustration of an example of phased sequencing processes.

FIG. 8 provides a schematic illustration of an example subset of the genome of a healthy patient (top panel) and a cancer patient with a gain in haplotype copy number (central panel) or loss of haplotype copy number (bottom panel).

FIG. 9A illustrates a schematic illustration showing a relative contribution of tumor DNA. FIG. 9B illustrates a representation of detecting such copy gains and losses by ordinary sequencing methods.

FIG. 10 provides a schematic illustration of an example of detecting copy gains and losses using a single variant position (left panel) and combined variant positions (right panel).

FIG. 11 provides a schematic illustration of the potential of described methods and systems to identify gains and losses in copy number.

FIG. 12 illustrates an example workflow for performing an aneuploidy test based on determination of chromosome number and copy number variation using methods and compositions described herein.

FIGS. 13A-B illustrate an example overview of a process for identifying structural variations such as translocations and gene fusions in genetic samples. FIG. 13A illustrates an example of identification of a non-translocated genotype. FIG. 13B illustrates an example of identification of a translocated genotype.

FIG. 14 schematically depicts an example workflow of analyzing a paternal nucleic acid sequence as described herein.

FIG. 15 schematically depicts an example workflow of analyzing a maternal nucleic acid sequence as described herein.

FIG. 16 schematically depicts an example workflow of analyzing a fetal nucleic acid sequence as described herein.

FIG. 17 schematically depicts an example workflow of analyzing a reference nucleic acid sequence as described herein.

FIG. 18 schematically depicts an example workflow of analyzing a sample nucleic acid sequence as described herein.

FIG. 19 shows an example of a microfluidic channel structure for partitioning individual biological particles.

FIG. 20 shows an example of a microfluidic channel structure for delivering barcode carrying beads to droplets.

FIG. 21 shows an example of a microfluidic channel structure for co-partitioning biological particles and reagents.

FIG. 22 shows an example of a microfluidic channel structure for the controlled partitioning of beads into discrete droplets.

FIG. 23 shows an example of a microfluidic channel structure for increased droplet generation throughput.

FIG. 24 shows another example of a microfluidic channel structure for increased droplet generation throughput.

FIGS. 25A-B illustrate another example of a microfluidic channel structure with a geometric feature for controlled partitioning. FIG. 25A shows a cross-section view of the microfluidic channel structure. FIG. 25B shows a perspective view of the channel structure of FIG. 25A.

FIG. 26 illustrates an example of a barcode carrying bead.

FIG. 27 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle,” as used herein, generally refers to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

As used herein, the term “organism” generally refers to a contiguous living system. Non-limiting examples of organisms includes animals (e.g., humans, other types of mammals, birds, reptiles, insects, other example types of animals described elsewhere herein), plants, fungi and bacterium.

As used herein, the term “contig” generally refers to a contiguous nucleic acid sequence of a given length. The contiguous sequence may be derived from an individual sequence read, including either a short or long read sequence read, or from an assembly of sequence reads that are aligned and assembled based upon overlapping sequences within the reads, or that are defined as linked within a fragment based upon other known linkage data, e.g., the tagging with common barcodes as described elsewhere herein. These overlapping sequence reads may likewise include short reads, e.g., less than 500 bases, e.g., in some cases from approximately 100 to 500 bases, and in some cases from 100 to 250 bases, or based upon longer sequence reads, e.g., greater than 500 bases, 1000 bases or even greater than 10,000 bases.

### Overview

This disclosure provides methods and systems useful in providing significant advances in the characterization of genetic material. In some cases, the methods and systems can be useful in providing genetic characterizations that are very difficult or even impossible using generally available technologies, including, for example, haplotype phasing, identifying structural variations, e.g., deletions, duplications, copy-number variants, insertions, inversions, retrotransposons, translocations, LTRs, STRs, and a variety of other useful characterizations. In some cases, the disclosure provides methods and systems useful in characterizing nucleic acid sequence information derived from a biological sample.

The nucleic acid molecules described herein can be nucleic acid molecules derived from a biological sample. In some embodiments, the biological sample is a maternal biological sample and/or a paternal biological sample. For example, the maternal biological sample is a maternal cell-free biological sample. The maternal cell-free biological sample can comprise nucleic acid molecules derived from a maternal source and nucleic acid molecules derived from a fetal source. The maternal biological sample or paternal biological sample can be a whole blood sample or a tissue sample. The maternal biological sample or paternal biological sample can be a buffy coat sample from said whole blood sample. The maternal cell-free biological sample can be a plasma sample. The biological sample can comprise at least about 1 ng of DNA. The biological sample can comprise at least about 1 ng, 5 ng, 10 ng, 50 ng, or 100 ng, or more, of DNA. In some cases, when the biological sample is plasma, the biological sample is less than 1 mL. Alternatively, when the biological sample is plasma, the biological sample can be about 1 mL, 1.5 mL, 2 mL, 2.5 mL, 3 mL, or greater than 3 mL.

In general, the methods and systems described herein accomplish the above goals by providing for the sequencing of long individual nucleic acid molecules, which permit the identification and use of long range variant information, e.g., relating variations to different sequence segments, including sequence segments containing other variations, that are separated by significant distances in the originating sequence, e.g., longer than is provided by short read sequencing technologies. However, these methods and systems achieve these objectives with the advantage of extremely low sequencing error rates of short read sequencing technologies, and far below those of the reported long read-length sequencing technologies, e.g., single molecule sequencing, such as SMRT Sequencing and nanopore sequencing technologies.

In general, the methods and systems described herein segment long nucleic acid molecules into smaller fragments that are sequenceable using high-throughput, higher accuracy short-read sequencing technologies, but do such segmentation in a manner that allows the sequence information derived from the smaller fragments to be attributed to the originating longer individual nucleic acid molecules. By enriching for specific target regions of the parental genome, sequencing efficiency and depth of coverage can be increased compared to a counterpart non-enriched sample. By attributing sequence reads to an originating longer nucleic acid molecule, one can gain significant characterization information for that longer nucleic acid sequence that one cannot generally obtain from short sequence reads alone. As noted, such characterization information can include haplotype phasing, identification of structural variations, and identifying copy number variations.

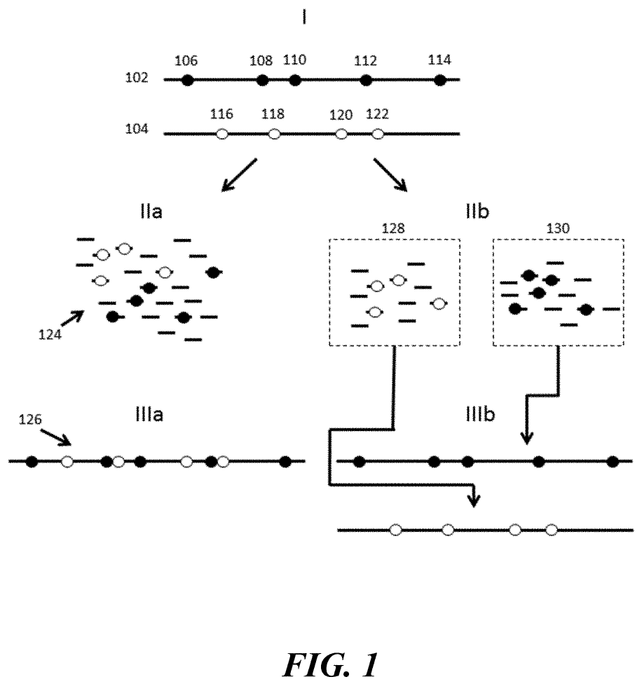

The advantages of the methods and systems described herein are described with respect to a number of general examples. In a first example, phased sequence variants are identified and characterized using the methods and systems described herein. FIG. 1 schematically illustrates the challenges of phased variant calling and the solutions presented by the methods described herein. As shown, nucleic acids 102 and 104 in Panel I represent two haploid sequences of the same region of different chromosomes, e.g., maternally and paternally inherited chromosomes. Each sequence includes a series of variants, e.g., variants 106-114 on nucleic acid 102, and variants 116-122 on nucleic acid 104, at different alleles that characterize each haploid sequence. Because of their very short sequence reads, most sequencing technologies are unable to provide the context of individual variants relative to other variants on the same haploid sequence. Additionally, because they rely on sample preparation techniques that do not separate individual molecular components, e.g., each haploid sequence, one is unable to identify the phasing of the various variants, e.g., the haploid sequence from which a variant derives. As a result, these short read technologies are unable to resolve these variants to their originating molecules. The difficulties with this approach are schematically illustrated in Panels IIa and IIIa. Briefly, pooled fragments from both haploid sequences, shown in Panel IIa, are sequenced, resulting in a large number of short sequence reads 124, and the resulting sequence 126 is assembled (shown in Panel IIIa). As shown, because one does not have the relative phasing context of any of the shorter sequence reads in Panel IIa, one may be unable to resolve the variants as between two different haploid sequences in the assembly process. Accordingly, the resulting assembly shown in Panel IIIa, results in single consensus sequence assembly 126, including all of variants 106-122.

In contrast, and as shown in Panel IIb of FIG. 1, the methods and systems described herein breakdown or segment the longer nucleic acids 102 and 104 into shorter, sequenceable fragments, as with the above described approach, but retain with those fragments the ability to attribute them to their originating molecular context. This is schematically illustrated in Panel IIb, in which different fragments are grouped or “compartmentalized” according to their originating molecular context. In the context of the disclosure, this grouping can be accomplished through one or both of physically partitioning the fragments into groups that retain the molecular context, as well as tagging those fragments in order to subsequently be able to elucidate that context.

This grouping is schematically illustrated as the allocation of the shorter sequence reads as between groups 128 and 130, representing short sequence reads from nucleic acids 102 and 104, respectively. Because the originating sequence context is retained through the sequencing process, one can employ that context in resolving the original molecular context, e.g., the phasing, of the various variants 106-114 and 116-122 as between sequences 102 and 104, respectively.

In another example advantaged application, the methods and systems are useful in characterizing structural variants that are generally unidentifiable or at least difficult to identify, using short read sequence technologies.

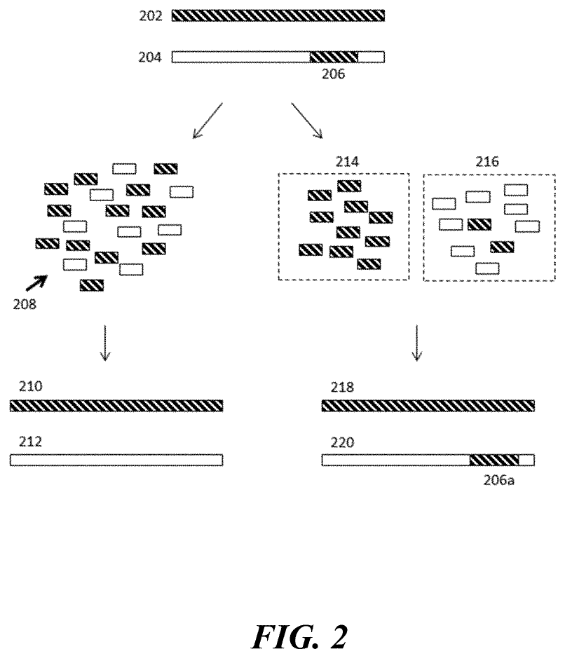

This is schematically illustrated with reference to a simple translocation event in FIG. 2. As shown, a genomic sample may include nucleic acids that include a translocation event, e.g., a translocation of genetic element 206 from sequence 202 to sequence 204. Such translocations may be any of a variety of different translocation types, including, for example, translocations between different chromosomes, whether to the same or different regions, between different regions of the same chromosome.

Again, as with the example illustrated in FIG. 1, above, conventional sequencing starts by breaking up the sequences 202 and 204 in Panel I into small fragments and producing short sequence reads 208 from those fragments, as shown in Panel IIa. Because these sequence fragments 208 are relatively short, the context of the translocated sequence 206, i.e., as originating from a variant location on the same or a different sequence, is easily lost during the assembly process. Further, because of their short read lengths, sequence assemblies are often predicated on the use of a reference sequence that would, almost by definition, not reflect structural variations. As such, the short sequence reads 208 would invariably be assembled to disregard the proper location of the translocated sequence 206, and would instead assemble the non-variant sequences 210 and 212, as shown in Panel IIIa.

In contrast, using the methods and systems described herein, the short sequence reads derived from sequences 202 and 204, are provided with a compartmentalization, shown in Panel IIb as groups 214 and 216, that retain the original molecular grouping of the smaller sequence fragments, allowing their assembly as sequences 218 and 220, shown in Panel IIIb allowing attribution back to the originating sequences 202 and 204, and identification of the translocation variation, e.g., translocated sequence segment 206a in correct sequence assemblies 218 and 220, as illustrated in Panel IIIb.

As noted above, the methods and systems described herein provide individual molecular context for short sequence reads of longer nucleic acids. As used herein, individual molecular context refers to sequence context beyond the specific sequence read, e.g., relation to adjacent or proximal sequences, that are not included within the sequence read itself, and as such, will generally be such that they may not be included in whole or in part in a short sequence read, e.g., a read of about 150 bases, or about 300 bases for paired reads. In some aspects, the methods and systems provide long range sequence context for short sequence reads. Such long range context includes relationship or linkage of a given sequence read to sequence reads that are within a distance of each other of longer than 1 kilobase (kb), longer than 5 kb, longer than 10 kb, longer than 15 kb, longer than 20 kb, longer than 30 kb, longer than 40 kb, longer than 50 kb, longer than 60 kb, longer than 70 kb, longer than 80 kb, longer than 90 kb or even longer than 100 kb, or longer. By providing longer range individual molecular context, the methods and systems described herein also provide much longer inferred molecular context. Sequence context, as described herein, can include lower resolution context, e.g., from mapping the short sequence reads to the individual longer molecules or contigs of linked molecules, as well as the higher resolution sequence context, e.g., from long range sequencing of large portions of the longer individual molecules, e.g., having contiguous determined sequences of individual molecules where such determined sequences are longer than 1 kb, longer than 5 kb, longer than 10 kb, longer than 15 kb, longer than 20 kb, longer than 30 kb, longer than 40 kb, longer than 50 kb, longer than 60 kb, longer than 70 kb, longer than 80 kb, longer than 90 kb or even longer than 100 kb. As with sequence context, the attribution of short sequences to longer nucleic acids, e.g., both individual long nucleic acid molecules or collections of linked nucleic acid molecules or contigs, may include both mapping of short sequences against longer nucleic acid stretches to provide high level sequence context, as well as providing assembled sequences from the short sequences through these longer nucleic acids. Furthermore, while one may utilize the long range sequence context associated with long individual molecules, having such long range sequence context also allows one to infer even longer range sequence context. By way of one example, by providing the long range molecular context described above, one can identify overlapping variant portions, e.g., phased variants, translocated sequences, etc., among long sequences from different originating molecules, allowing the inferred linkage between those molecules. Such inferred linkages or molecular contexts are referred to herein as “inferred contigs.” In some cases when discussed in the context of phased sequences, the inferred contigs may represent commonly phased sequences, e.g., where by virtue of overlapping phased variants, one can infer a phased contig of substantially greater length than the individual originating molecules. These phased contigs are referred to herein as “phase blocks.”

By starting with longer single molecule reads, one can derive longer inferred contigs or phase blocks than may otherwise be attainable using short read sequencing technologies or other approaches to phased sequencing. See, e.g., published U.S. Patent Publication No. 2013/0157870, the full disclosure of which is herein incorporated by reference in its entirety. In particular, using the methods and systems described herein, one can obtain inferred contig or phase block lengths having an N50 (the contig or phase block length for which the collection of all phase blocks or contigs of that length or longer contain at least half of the sum of the lengths of all contigs or phase blocks, and for which the collection of all contigs or phase blocks of that length or shorter also contains at least half the sum of the lengths of all contigs or phase blocks), mode, mean, or median of at least about 10 kilobases (kb), at least about 20 kb, at least about 50 kb. In some aspects, inferred contig or phase block lengths have an N50, mode, mean, or median of at least about 100 kb, at least about 150 kb, at least about 200 kb, and in some cases, at least about 250 kb, at least about 300 kb, at least about 350 kb, at least about 400 kb, and in some cases, at least about 500 kb, at least about 750 kb, at least about 1 Mb, at least about 1.75 Mb, at least about 2.5 Mb or more, are attained. In still other cases, maximum inferred contig or phase block lengths of at least or in excess of 20 kb, 40 kb, 50 kb, 100 kb, 200 kb, 300 kb, 400 kb, 500 kb, 750 kb, 1 megabase (Mb), 1.75 Mb, 2 Mb or 2.5 Mb may be obtained. In still other cases, inferred contigs or phase blocks lengths can be at least about 20 kb, at least about 40 kb, at least about 50 kb, at least about 100 kb, at least about 200 kb, and in some cases, at least about 500 kb, at least about 750 kb, at least about 1 Mb, and in some cases at least about 1.75 Mb, at least about 2.5 Mb or more.

In one aspect, the methods and systems described herein provide for the compartmentalization, depositing, or partitioning of sample nucleic acids, or fragments thereof, into discrete compartments or partitions (referred to interchangeably herein as partitions), where each partition maintains separation of its own contents from the contents of other partitions. Unique identifiers, e.g., barcodes, may be previously, subsequently or concurrently delivered to the partitions that hold the compartmentalized or partitioned sample nucleic acids, in order to allow for the later attribution of the characteristics, e.g., nucleic acid sequence information, to the sample nucleic acids included within a particular compartment, and particularly to relatively long stretches of contiguous sample nucleic acids that may be originally deposited into the partitions. Nucleic acids tagged with unique identifiers can then be enriched for target sequences of interest (e.g., whole exome) prior to further processing and/or nucleic acid sequencing and analysis.

The sample nucleic acids can be partitioned such that the nucleic acids are present in the partitions in relatively long fragments or stretches of contiguous nucleic acid molecules, also referred to herein as a long nucleic acid molecule. These fragments can represent a number of overlapping fragments of the overall sample nucleic acids to be analyzed, e.g., an entire chromosome, exome, or other large genomic fragment. These sample nucleic acids may include whole genomes, individual chromosomes, exomes, amplicons, or any of a variety of different nucleic acids of interest. In some cases, these fragments of the sample nucleic acids may be longer than 100 bases, longer than 500 bases, longer than 1 kb, longer than 5 kb, longer than 10 kb, longer than 15 kb, longer than 20 kb, longer than 30 kb, longer than 40 kb, longer than 50 kb, longer than 60 kb, longer than 70 kb, longer than 80 kb, longer than 90 kb, or even longer than 100 kb, which permits the longer range molecular context described above. In some cases, a plurality of partitions is generated. A given partition of the plurality of partitions can comprise a long nucleic acid molecule from a plurality of nucleic acid molecules derived from the biological sample. The biological sample can be a maternal or paternal biological sample. In some embodiments, the maternal biological sample is a maternal cell free sample from a pregnant woman that comprises both maternal and fetal nucleic acid sequences.

The sample nucleic acids can also be partitioned at a level whereby a given partition has a very low probability of including two overlapping fragments of the starting sample nucleic acid. This can be accomplished by providing the sample nucleic acid at a low input amount and/or concentration during the partitioning process. As a result, in some cases, a given partition may include a number of long, but non-overlapping fragments of the starting sample nucleic acids. The sample nucleic acids in the different partitions are then associated with unique identifiers, where for any given partition, nucleic acids contained therein possess the same unique identifier, but where different partitions may include different unique identifiers. Moreover, because the partitioning allocates the sample components into very small volume partitions or droplets, it will be appreciated that in order to achieve the allocation as set forth above, one need not conduct substantial dilution of the sample, as may be required in higher volume processes, e.g., in tubes, or wells of a multiwell plate. Further, because the systems described herein employ such high levels of barcode diversity, one can allocate diverse barcodes among higher numbers of genomic equivalents, as provided above. In particular, previously described, multiwell plate approaches (see, e.g., U.S. Patent Publication No. 2013/0079231 and 2013/0157870, the full disclosures of which are herein incorporated by reference in their entireties) may only operate with a hundred to a few hundred different barcode sequences, and employ a limiting dilution process of their sample in order to be able to attribute barcodes to different cells/nucleic acids. As such, they generally operate with far fewer than 100 cells, which can provide a ratio of genomes:(barcode type) on the order of 1:10, and certainly well above 1:100. The systems described herein, on the other hand, because of the high level of barcode diversity, e.g., in excess of 10,000, 100,000, 500,000, etc. diverse barcode types, can operate at genome:(barcode type) ratios that are on the order of 1:50 or less, 1:100 or less, 1:1000 or less, or even smaller ratios, while also allowing for loading higher numbers of genomes (e.g., on the order of greater than 100 genomes per assay, greater than 500 genomes per assay, 1000 genomes per assay, or even more) while still providing for far improved barcode diversity per genome.

Often, the sample is combined with a set of oligonucleotide tags that are releasably-attached to beads prior to the partitioning. The oligonucleotides may comprise at least a first and second region. The first region may be a barcode region that, as between oligonucleotides within a given partition, may comprise substantially the same barcode sequence, but as between different partitions, may and, in most cases, comprise a different barcode sequence. The second region may be an N-mer (e.g., a random N-mer or a sequence designed to target a particular sequence) that can be used to prime the nucleic acids within the sample within the partitions. In some cases, where the N-mer is designed to target a particular sequence, it may be designed to target a particular chromosome (e.g., chromosome 1, 13, 18, or 21), or region of a chromosome, e.g., an exome or other targeted region. In some cases, the N-mer may be designed to target a particular gene or genetic region, such as a gene or region associated with a disease or disorder (e.g., cancer). Within the partitions, an amplification reaction may be conducted using the N-mer sequence to prime the nucleic acid sample at different places along the length of the nucleic acid. As a result of the amplification, each partition may contain amplified products of the nucleic acid that are attached to an identical or near-identical barcode, and that may represent overlapping, smaller fragments of the nucleic acids in each partition. The barcode can serve as a marker that signifies that a set of nucleic acids originated from the same partition, and thus potentially also originated from the same strand of nucleic acid. In some embodiments, when sample nucleic acids are amplified by random N-mers, following amplification, select regions of the amplified nucleic acid fragments are targeted (e.g., by nucleic acid capture) to enrich for sequences of interest (e.g., whole exome or other sequences of interest) in the amplified nucleic acid fragments. In other embodiments, when sample nucleic acids are amplified by N-mers targeted to one or more specific sequence, select regions of the sample nucleic acid are targeted in the partition by an amplification reaction to enrich for sequences of interest. Following amplification, the amplified nucleic acids may be released from the partition, pooled, sequenced, aligned using one or more sequencing algorithms, and further analyzed for genetic features of interest (e.g., relative haplotype dosing (RHDO)). Because shorter sequence reads may, by virtue of their associated barcode sequences, be aligned and attributed to a long fragment of the sample nucleic acid, all of the identified variants on that sequence can be attributed to an originating fragment and originating chromosome. Further, by aligning multiple co-located variants across multiple long fragments, one can further characterize that chromosomal contribution. Accordingly, conclusions regarding the phasing of particular genetic variants may then be drawn. Such information may be useful for identifying haplotypes, which are generally a specified set of genetic variants that reside on the same nucleic acid strand or on different nucleic acid strands. Copy number variations may also be identified in this manner.

The described methods and systems provide significant advantages over current nucleic acid sequencing technologies and their associated sample preparation methods. Haplotype phasing and copy number variation data may not be available by sequencing genomic DNA because biological samples (blood, cells, or tissue samples, for example) are processed en masse to extract the genetic material from an ensemble of cells, and convert it into sequencing libraries that are configured specifically for a given sequencing technology. As a result of this ensemble sample processing approach, sequencing data generally provides non-phased genotypes, in which it is not possible to determine whether genetic information is present on the same or different chromosomes.

In addition to the inability to attribute genetic characteristics to a particular chromosome, such ensemble sample preparation and sequencing methods are also predisposed towards primarily identifying and characterizing the majority constituents in the sample, and are not designed to identify and characterize minority constituents, e.g., genetic material contributed by one chromosome, or by one or a few cells, or fragmented tumor cell DNA molecule circulating in the bloodstream, that constitute a small percentage of the total DNA in the extracted sample. In contrast, the methods described herein provide targeted, phased nucleic acid sequence information from nucleic acid molecules present in a biological sample. Thus, instead of generating a phased whole genome sequencing library, a targeted phased sequencing library is generated allowing for decreased sequencing costs and/or increased sequencing depth thereby increasing the efficiency and quality of fetal mutation calls and/or CNVs.

The described methods and systems also provide a significant advantage for detecting minor populations that are present in a larger sample. As such, they can be useful for assessing copy number variations in a sample since often only a small portion of a clinical sample contains tissue with copy number variations. For example, if the sample is a blood sample from a pregnant woman, only a small fraction of the sample contains circulating cell-free fetal DNA.

The use of the barcoding technique disclosed herein confers the unique capability of providing individual molecular context for a given set of genetic markers, i.e., attributing a given set of genetic markers (as opposed to a single marker) to individual sample nucleic acid molecules, and through variant coordinated assembly, to provide a broader or even longer range inferred individual molecular context, among multiple sample nucleic acid molecules, and/or to a specific chromosome. These genetic markers may include specific genetic loci, e.g., variants, such as SNPs, or they may include short sequences. Furthermore, the use of barcoding confers the additional advantages of facilitating the ability to discriminate between minority constituents and majority constituents of the total nucleic acid population extracted from the sample, e.g., for detection and characterization of circulating cell-free fetal DNA in the bloodstream, and also reduces or eliminates amplification bias during any amplification. In addition, implementation in a microfluidics format confers the ability to work with extremely small sample volumes and low input quantities of DNA, as well as the ability to rapidly process large numbers of sample partitions (e.g., droplets) to facilitate genome-wide tagging.

As described previously, an advantage of the methods and systems described herein is that they can achieve results through the use of ubiquitously available, short read sequencing technologies. Such short read sequencing technologies have the advantages of being readily available and widely dispersed within the research community, with protocols and reagent systems that are well characterized and highly effective. These short read sequencing technologies include those available from, e.g., Illumina, Inc. (e.g., GXII, NextSeq, MiSeq, HiSeq, X10), Ion Torrent division of Thermo-Fisher (e.g., Ion Proton and Ion PGM), pyrosequencing methods, as well as others.

Of particular advantage is that the methods and systems described herein utilize these short read sequencing technologies and do so with their associated low error rates. In particular, the methods and systems described herein achieve individual molecular read lengths or context, as described above, but with individual sequencing reads, excluding mate pair extensions, that are shorter than 1,000 bp, shorter than 500 bp, shorter than 300 bp, shorter than 200 bp, shorter than 150 bp or even shorter; and with sequencing error rates for such individual molecular read lengths that are less than 5%, less than 1%, less than 0.5%, less than 0.1%, less than 0.05%, less than 0.01%, less than 0.005%, or even less than 0.001%.

### Work Flow Overview

In one example aspect, the methods and systems described in the disclosure provide for depositing or partitioning individual samples (e.g., nucleic acids) into discrete partitions, where each partition maintains separation of its own contents from the contents in other partitions. As used herein, the partitions refer to containers or vessels that may include a variety of different forms, e.g., droplet emulsions, wells, tubes, micro or nanowells, through holes, or the like. In some aspects, however, the partitions are flowable within fluid streams. These vessels may be comprised of, e.g., microcapsules or micro-vesicles that have an outer barrier surrounding an inner fluid center or core, or they may be a porous matrix that is capable of entraining and/or retaining materials within its matrix. In some aspects, however, these partitions may comprise droplets of aqueous fluid within a non-aqueous continuous phase, e.g., an oil phase. A variety of different vessels are described in, for example, U.S. Patent Publication No. 2014/0155295, filed Aug. 13, 2013. Likewise, emulsion systems for creating stable droplets in non-aqueous or oil continuous phases are described in detail in, e.g., U.S. Patent Publication No. 2010/0105112, the full disclosure of which is herein incorporated by reference in its entirety. In certain cases, microfluidic channel networks can be suited for generating partitions as described herein. Examples of such microfluidic devices include those described in detail in U.S. Pat. No. 9,694,361, filed Apr. 9, 2015, the full disclosure of which is incorporated herein by reference in its entirety for all purposes. Alternative mechanisms may also be employed in the partitioning of individual cells, including porous membranes through which aqueous mixtures of cells are extruded into non-aqueous fluids. Such systems are generally available from, e.g., Nanomi, Inc.

In the case of droplets in an emulsion, partitioning of sample materials, e.g., nucleic acids, into discrete partitions may generally be accomplished by flowing an aqueous, sample containing stream, into a junction into which is also flowing a non-aqueous stream of partitioning fluid, e.g., a fluorinated oil, such that aqueous droplets are created within the flowing stream partitioning fluid, where such droplets include the sample materials. As described below, the partitions, e.g., droplets, can also include co-partitioned barcode oligonucleotides. The relative amount of sample materials within any particular partition may be adjusted by controlling a variety of different parameters of the system, including, for example, the concentration of sample in the aqueous stream, the flow rate of the aqueous stream and/or the non-aqueous stream, and the like. The partitions described herein are often characterized by having extremely small volumes. For example, in the case of droplet based partitions, the droplets may have overall volumes that are less than 1000 picoliters (pL), less than 900 pL, less than 800 pL, less than 700 pL, less than 600 pL, less than 500 pL, less than 400 pL, less than 300 pL, less than 200 pL, less than 100 pL, less than 50 pL, less than 20 pL, less than 10 pL, or even less than 1 pL. Where co-partitioned with beads, it will be appreciated that the sample fluid volume within the partitions may be less than 90% of the above described volumes, less than 80%, less than 70%, less than 60%, less than 50%, less than 40%, less than 30%, less than 20%, or even less than 10% the above described volumes. In some cases, the use of low reaction volume partitions can be advantageous in performing reactions with very small amounts of starting reagents, e.g., input nucleic acids. Methods and systems for analyzing samples with low input nucleic acids are presented in U.S. Patent Publication No. 2015/0376605, filed Jun. 26, 2015, the full disclosure of which is hereby incorporated by reference in its entirety.

Once the samples are introduced into their respective partitions, in accordance with the methods and systems described herein, the sample nucleic acids within partitions are generally provided with unique identifiers such that, upon characterization of those nucleic acids they may be attributed as having been derived from their respective origins. Accordingly, the sample nucleic acids can be co-partitioned with the unique identifiers (e.g., barcode sequences). In some aspects, the unique identifiers are provided in the form of oligonucleotides that comprise nucleic acid barcode sequences that may be attached to those samples. The oligonucleotides are partitioned such that as between oligonucleotides in a given partition, the nucleic acid barcode sequences contained therein are the same, but as between different partitions, the oligonucleotides can have differing barcode sequences. In some aspects, only one nucleic acid barcode sequence may be associated with a given partition, although in some cases, two or more different barcode sequences may be present.

The nucleic acid barcode sequences can include from 6 to about 20 or more nucleotides within the sequence of the oligonucleotides. These nucleotides may be completely contiguous, i.e., in a single stretch of adjacent nucleotides, or they may be separated into two or more separate subsequences (e.g., barcode sequence segments) that are separated by one or more nucleotides. In some cases, separated subsequences may be from about 4 to about 16 nucleotides in length.

The co-partitioned oligonucleotides can also comprise other functional sequences useful in the processing of the partitioned nucleic acids. These sequences include, e.g., targeted or random/universal amplification primer sequences for amplifying the genomic DNA from the individual nucleic acids within the partitions while attaching the associated barcode sequences, sequencing primers, hybridization or probing sequences, e.g., for identification of presence of the sequences, or for pulling down barcoded nucleic acids, or any of a number of other potential functional sequences. Again, co-partitioning of oligonucleotides and associated barcodes and other functional sequences along with sample material is described in, for example, U.S. Patent Publication No. 2014/0378345, filed on Jun. 26, 2014, as well as U.S. Pat. No. 9,644,204, filed Feb. 7, 2014, the full disclosures of which is hereby incorporated by reference in their entireties.

Briefly, in one example process, beads are provided that each include large numbers of the above described oligonucleotides releasably attached to the beads, where all of the oligonucleotides attached to a particular bead include the same nucleic acid barcode sequence, but where a large number of diverse barcode sequences may be represented across the population of beads used. In some cases, the population of beads provides a diverse barcode sequence library that includes at least 1,000 different barcode sequences, at least 10,000 different barcode sequences, at least 100,000 different barcode sequences, or in some cases, at least 1,000,000 different barcode sequences. Additionally, each bead may be provided with large numbers of oligonucleotide molecules attached. In particular, the number of oligonucleotide molecules comprising the barcode sequence on an individual bead may be at least about 10,000 oligonucleotides, at least 100,000 oligonucleotide molecules, at least 1,000,000 oligonucleotide molecules, at least 100,000,000 oligonucleotide molecules, and in some cases at least 1 billion oligonucleotide molecules.

In some embodiments, the barcode oligonucleotides are releasable from the beads upon the application of a particular stimulus to the beads. In some cases, the stimulus may be a photo-stimulus, e.g., through cleavage of a photo-labile linkage that releases the oligonucleotides. In some cases, a thermal stimulus may be used, where elevation of the temperature of the bead environment results in cleavage of a linkage or otherwise causes the release of the oligonucleotides from the beads. In some cases, a chemical stimulus may be used that cleaves a linkage of the oligonucleotides to the beads, or otherwise results in release of the oligonucleotides from the beads.

In accordance with the methods and systems described herein, the beads including the attached oligonucleotides may be co-partitioned with the individual samples, such that a single bead and a sample (e.g., a single HMW DNA molecule) are contained within an individual partition. In some cases, where single bead partitions are desired, the relative flow rates of the fluids can be controlled such that, on average, the partitions contain less than one bead per partition in order to ensure that the partitions are primarily singly occupied. Likewise, one may wish to control the flow rate to provide that a higher percentage of partitions are occupied, e.g., allowing for only a small percentage of unoccupied partitions. In some aspects, the flows and channel architectures are controlled as to ensure a desired number of singly occupied partitions are less than a certain level of unoccupied partitions, and/or less than a certain level of multiply occupied partitions.

In some embodiments, for example, nucleic acid molecules from a biological sample (e.g., a maternal cell free nucleic acid sample) and a plurality of beads comprising a plurality of nucleic acid barcode molecules releasable attached thereto are partitioned such that at least some partitions contain: (a) a high molecular weight (HMW) nucleic acid molecule from the biological sample (e.g., a single HMW nucleic acid molecule); and (b) a single bead comprising nucleic acid barcode molecules comprising (i) a common barcode sequence, and (ii) a random N-mer sequence. The HMW nucleic acid molecule can range from about 10 kb to over 100 kb in size. In some instances, the HMW nucleic acid molecule is at least 10 kb, 20 kb, 30 kb, 40 kb, 50 kb, 60 kb, 70 kb, 80 kb, 90 kb, or 100 kb in length. In other cases, the HMW nucleic acid molecule is over 100 kb in length. In each partition, before or after releasing the barcode oligonucleotides from the beads, barcode oligonucleotides from the beads are utilized to generate a set of barcoded nucleic acid fragments derived from the HMW nucleic acid molecule. The barcoded nucleic acid molecules or derivatives thereof may then be enriched (e.g., by nucleic acid capture) to generate an enriched set of barcoded nucleic acid molecules. The enriched barcoded nucleic acid molecules can subsequently be processed to generate a nucleic acid sequencing library and sequenced to generate nucleic acid sequence information. The nucleic acid sequence information can be maternal, maternal/fetal, or paternal nucleic acid sequence information of the nucleic acid molecules derived from the biological sample (e.g., maternal and fetal sequence information derived from a maternal cell free nucleic acid sample from a pregnant female).

In some cases, enriching the barcoded sample nucleic acid molecules is performed using nucleic acid capture. The nucleic acid capture can comprise nucleic acid capture of one or more target regions in the plurality of barcoded nucleic acid molecules. The one or more target regions can comprise a particular gene or targeted gene panel. The one or more target regions can be one or more regions of the genome indicative of a disease or condition. The disease or condition can be a disease or condition of the fetus. The disease or condition can be caused by a genetic variation. The genetic variation can be an aneuploidy, structural variation, copy number variation, single nucleotide variant (SNV), or a combination thereof. The nucleic acid capture can be transcriptome capture. The nucleic acid capture can be exome capture, also referred to herein as exome enrichment. Exome enrichment can be performed using any suitable methodology, such as using an Agilent SureSelect kit. In some embodiments, the nucleic acid capture comprises selectively capturing the one or more target regions via nucleic acid hybridization. The hybridization can be hybridization of the one or more target regions to one or more complementary probes. In some cases, the enriching is performed prior to barcoding of the nucleic acid molecules. In some cases, the enriching is performed after barcoding of the nucleic acid molecules. In some cases, the sequencing is performed after the enriching.

In some cases, enriching the barcoded nucleic acid molecules is performed using nucleic acid amplification using primers designed to amplify one or more target regions from the plurality of barcoded nucleic acid molecules to yield amplified, targeted barcoded nucleic acid molecules.

In other cases, nucleic acid molecules from a biological sample (e.g., a maternal cell free nucleic acid sample) and a plurality of beads comprising a plurality of nucleic acid barcode molecules releasable attached thereto are partitioned such that at least some partitions contain: (1) a high molecular weight (HMW) nucleic acid molecule from the biological sample; and (2) a single bead comprising nucleic acid barcode molecules comprising (i) a common barcode sequence, and (ii) one or more primer sequences targeting one or more regions in the sample nucleic acid molecules. In each partition, before or after releasing the barcode oligonucleotides from the beads, barcode oligonucleotides from the beads are utilized to generate a set of targeted, barcoded nucleic acid fragments derived from the HMW nucleic acid molecule. In other embodiments, the one or more primer sequences targeting one or more regions in the sample nucleic acid are not present in the barcode oligonucleotide molecules, but instead, are contained in separate nucleic acid molecules (e.g., primers) that are partitioned with the BMW DNA and single bead.

Pooling of the barcoded oligonucleotides from each partition can create an oligonucleotide library. The nucleic acid capture can occur on the oligonucleotide library after the pooling to produce a targeted oligonucleotide library. In some embodiments, targeted PCR amplification is performed on the oligonucleotide library or the targeted oligonucleotide library to enrich for one or more target regions. Sequencing can be performed on the oligonucleotide library, the targeted oligonucleotide library, or the targeted PCR amplification products, or derivatives thereof. The sequencing can comprise sequencing to a depth of at least about 50×, 100×, 150×, 200×, 250×, or 300×, or more.

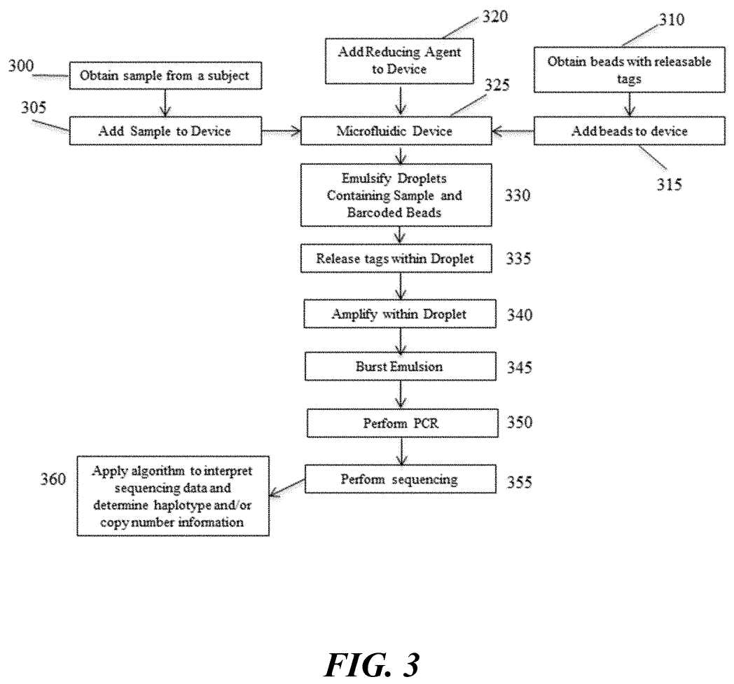

FIG. 3 illustrates an example method for barcoding and subsequently sequencing a sample nucleic acid, such as for use in a copy number variation or haplotype assay. First, a sample comprising nucleic acid may be obtained from a source, 300, and a set of barcoded beads may also be obtained, 310. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, a low quantity of the sample comprising nucleic acid, 305, barcoded beads, 315, and, in some cases, other reagents, e.g., a reducing agent, 320, are combined and subject to partitioning. By way of example, such partitioning may involve introducing the components to a droplet generation system, such as a microfluidic device, 325. With the aid of the microfluidic device 325, a water-in-oil emulsion 330 may be formed, where the emulsion contains aqueous droplets that contain sample nucleic acid, 305, reducing agent, 320, and barcoded beads, 315. The reducing agent may dissolve or degrade the barcoded beads, thereby releasing the oligonucleotides with the barcodes and random N-mers from the beads within the droplets, 335. The random N-mers may then prime different regions of the sample nucleic acid, resulting in amplified copies of the sample after amplification, where each copy is tagged with a barcode sequence, 340. In other cases, the oligonucleotides with the barcodes have a primer sequence directed to a specific target(s) of interest (e.g., a specific gene, locus, or whole exome). In some cases, each droplet contains a set of oligonucleotides that contain identical barcode sequences and different random N-mer sequences. Subsequently, the emulsion is broken, 345, and the barcoded sample nucleic acid fragments can be enriched for particular targets of interest. For example, barcoded sample fragments can be targeted by nucleic acid capture (e.g., hybridization to capture probes) to enrich for sequences of interest (e.g., the whole exome). In other cases, barcoded sample nucleic acid fragments can be enriched by nucleic acid amplification using primers directed to sequences of interest. Subsequent (or prior to enrichment), additional sequences (e.g., sequences that aid in particular sequencing methods, additional barcodes, etc.) may be added, via, for example, amplification methods, 350 (e.g., PCR). Sequencing may then be performed, 355, and an algorithm applied to interpret the sequencing data, 360. Sequencing algorithms are generally capable, for example, of performing analysis of barcodes to align sequencing reads and/or identify the sample from which a particular sequence read belongs. Further analysis on the sequencing reads can then be performed to identify phasing information and variant analysis (e.g., using RHDO or other methods described herein).

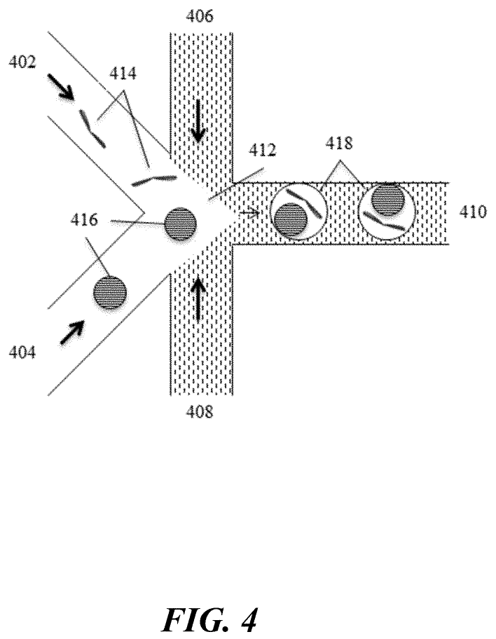

As noted above, while single bead occupancy may be desired, it will be appreciated that multiply occupied partitions, or unoccupied partitions may at times be present. An example of a microfluidic channel structure for co-partitioning samples and beads comprising barcode oligonucleotides is schematically illustrated in FIG. 4. As shown, channel segments 402, 404, 406, 408, and 410 are provided in fluid communication at channel junction 412. An aqueous stream comprising the individual samples 414 is flowed through channel segment 402 toward channel junction 412. As described elsewhere herein, these samples may be suspended within an aqueous fluid prior to the partitioning process.

Concurrently, an aqueous stream comprising the barcode carrying beads 416 is flowed through channel segment 404 toward channel junction 412. A non-aqueous partitioning fluid is introduced into channel junction 412 from each of side channels 406 and 408, and the combined streams are flowed into outlet channel 410. Within channel junction 412, the two combined aqueous streams from channel segments 402 and 404 are combined, and partitioned into droplets 418, that include co-partitioned samples 414 and beads 416. As noted previously, by controlling the flow characteristics of each of the fluids combining at channel junction 412, as well as controlling the geometry of the channel junction, one can optimize the combination and partitioning to achieve a desired occupancy level of beads, samples or both, within the partitions 418 that are generated.

As will be appreciated, a number of other reagents may be co-partitioned along with the samples and beads, including, for example, chemical stimuli, nucleic acid extension enzymes (e.g., polymerases), reverse transcription enzymes, and/or amplification reagents such as polymerases, reverse transcriptases, nucleoside triphosphates or NTP analogues, primer sequences and additional cofactors such as divalent metal ions used in such reactions, ligation reaction reagents, such as ligase enzymes and ligation sequences, dyes, labels, or other tagging reagents.

Once co-partitioned, the oligonucleotides disposed upon the bead may be used to barcode and amplify the partitioned samples. As utilized herein, the term “amplify” or “amplification” includes reactions such as polymerase chain reaction (PCR) and nucleic acid extension reaction (such as primer extension). An example process for use of these barcode oligonucleotides in amplifying and barcoding samples is described in detail in U.S. Patent Application Publication No. US2014/0378345, filed on Jun. 26, 2014, the full disclosures of which are hereby incorporated by reference in their entireties. Briefly, in one aspect, the oligonucleotides present on the beads that are co-partitioned with the samples and released from their beads into the partition with the samples. The oligonucleotides can include, along with the barcode sequence, a primer sequence at its 5′end. This primer sequence may be a random oligonucleotide sequence intended to randomly prime numerous different regions of the samples, or it may be a specific primer sequence targeted to prime upstream of a specific targeted region of the sample.

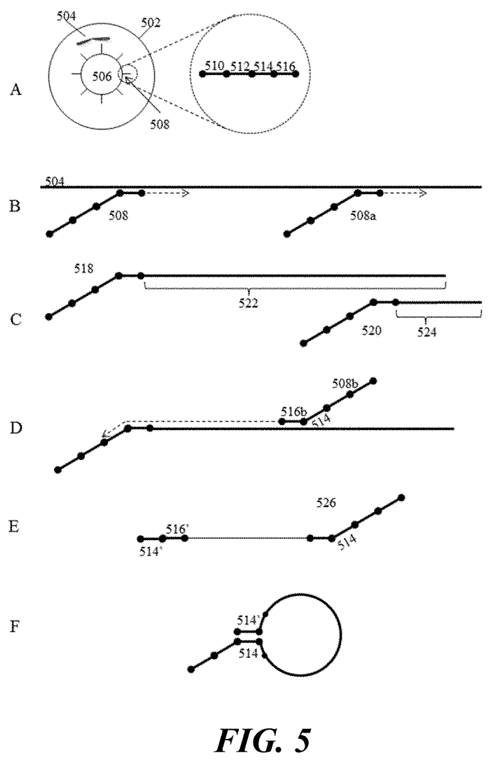

Once released, the primer portion of the oligonucleotide can anneal to a complementary region of the sample. Extension reaction reagents, e.g., a DNA polymerase, nucleoside triphosphates, co-factors (e.g., Mg²⁺ or Mn²⁺ etc.), that are also co-partitioned with the samples and beads, then extend the primer sequence using the sample as a template, to produce a complementary fragment to the strand of the template to which the primer annealed, with complementary fragment includes the oligonucleotide and its associated barcode sequence. Annealing and extension of multiple primers to different portions of the sample may result in a large pool of overlapping complementary fragments of the sample, each possessing its own barcode sequence indicative of the partition in which it was created. In some cases, these complementary fragments may themselves be used as a template primed by the oligonucleotides present in the partition to produce a complement of the complement that again, includes the barcode sequence. In some cases, this replication process is configured such that when the first complement is duplicated, it produces two complementary sequences at or near its termini, to allow the formation of a hairpin structure or partial hairpin structure that reduces the ability of the molecule to be the basis for producing further iterative copies. A schematic illustration of one example of this is shown in FIG. 5.

As the figure shows, oligonucleotides that include a barcode sequence are co-partitioned in, e.g., a droplet 502 in an emulsion, along with a sample nucleic acid 504. As noted elsewhere herein, the oligonucleotides 508 may be provided on a bead 506 that is co-partitioned with the sample nucleic acid 504, which oligonucleotides 508 can be releasable from the bead 506, as shown in panel A. The oligonucleotides 508 include a barcode sequence 512, in addition to one or more functional sequences, e.g., sequences 510, 514, and 516. For example, oligonucleotide 508 is shown as comprising barcode sequence 512, as well as sequence 510 that may function as an attachment or immobilization sequence for a given sequencing system, e.g., a P5 sequence used for attachment in flow cells of an Illumina Hiseq or Miseq system. As shown, the oligonucleotides also include a primer sequence 516, which may include a random or targeted N-mer for priming replication of portions of the sample nucleic acid 504. Also included within oligonucleotide 508 is a sequence 514 which may provide a sequencing priming region, such as a “read 1” or R1 priming region, that is used to prime polymerase mediated, template directed sequencing by synthesis reactions in sequencing systems. In some cases, the barcode sequence 512, immobilization sequence 510, and R1 sequence 514 may be common to all of the oligonucleotides attached to a given bead. The primer sequence 516 may vary for random N-mer primers, or may be common to the oligonucleotides on a given bead for certain targeted applications.

Based upon the presence of primer sequence 516, the oligonucleotides are able to prime the sample nucleic acid as shown in panel B, which allows for extension of the oligonucleotides 508 and 508a using polymerase enzymes and other extension reagents also co-portioned with the bead 506 and sample nucleic acid 504. As shown in panel C, following extension of the oligonucleotides that, for random N-mer primers, would anneal to multiple different regions of the sample nucleic acid 504; multiple overlapping complements or fragments of the nucleic acid are created, e.g., fragments 518 and 520. Although including sequence portions that are complementary to portions of sample nucleic acid, e.g., sequences 522 and 524, these constructs are generally referred to herein as comprising fragments of the sample nucleic acid 504, having the attached barcode sequences. As will be appreciated, the replicated portions of the template sequences as described above are often referred to herein as “fragments” of that template sequence. Notwithstanding the foregoing, however, the term “fragment” encompasses any representation of a portion of the originating nucleic acid sequence, e.g., a template or sample nucleic acid, including those created by other mechanisms of providing portions of the template sequence, such as actual fragmentation of a given molecule of sequence, e.g., through enzymatic, chemical or mechanical fragmentation. In some aspects, however, fragments of a template or sample nucleic acid sequence may denote replicated portions of the underlying sequence or complements thereof.

The barcoded nucleic acid fragments may then be subjected to characterization, e.g., through sequence analysis, or they may be further amplified in the process, as shown in panel D. For example, additional oligonucleotides, e.g., oligonucleotide 508b, also released from bead 306, may prime the fragments 518 and 520. In particular, again, based upon the presence of the random N-mer primer 516b in oligonucleotide 508b (which in some cases can be different from other random N-mers in a given partition, e.g., primer sequence 516), the oligonucleotide anneals with the fragment 518, and is extended to create a complement 526 to at least a portion of fragment 518 which includes sequence 528, that comprises a duplicate of a portion of the sample nucleic acid sequence. Extension of the oligonucleotide 508b continues until it has replicated through the oligonucleotide portion 508 of fragment 518. As noted elsewhere herein, and as illustrated in panel D, the oligonucleotides may be configured to prompt a stop in the replication by the polymerase at a desired point, e.g., after replicating through sequences 516 and 514 of oligonucleotide 508 that is included within fragment 518. As described herein, this may be accomplished by different methods, including, for example, the incorporation of different nucleotides and/or nucleotide analogues that are not capable of being processed by the polymerase enzyme used. For example, this may include the inclusion of uracil containing nucleotides within the sequence region 512 to prevent a non-uracil tolerant polymerase to cease replication of that region. As a result a fragment 526 is created that includes the full-length oligonucleotide 508b at one end, including the barcode sequence 512, the attachment sequence 510, the R1 primer region 514, and the random N-mer sequence 516b. At the other end of the sequence can be included the complement 516′ to the random N-mer of the first oligonucleotide 508, as well as a complement to all or a portion of the R1 sequence, shown as sequence 514′. The R1 sequence 514 and its complement 514′ are then able to hybridize together to form a partial hairpin structure 528. As will be appreciated because the random N-mers differ among different oligonucleotides, these sequences and their complements may not be expected to participate in hairpin formation, e.g., sequence 516′, which is the complement to random N-mer 516, would not be expected to be complementary to random N-mer sequence 516b. This may not be the case for other applications, e.g., targeted primers, where the N-mers may be common among oligonucleotides within a given partition.

By forming these partial hairpin structures, it allows for the removal of first level duplicates of the sample sequence from further replication, e.g., preventing iterative copying of copies. The partial hairpin structure also provides a useful structure for subsequent processing of the created fragments, e.g., fragment 526.

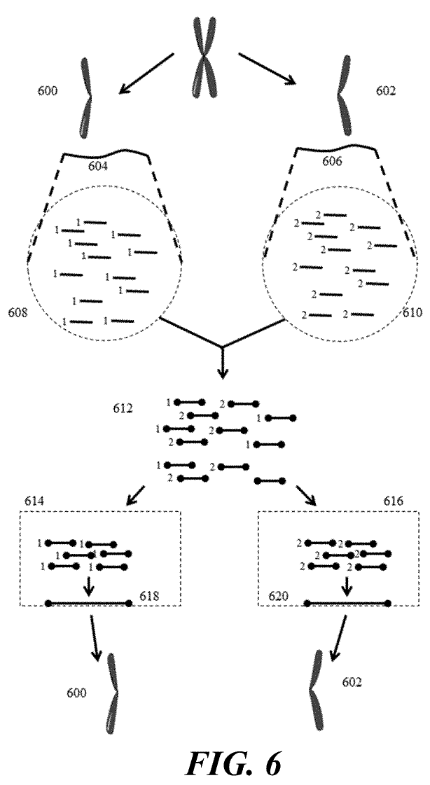

All of the fragments from multiple different partitions may then be pooled for sequencing on high throughput sequencers as described herein. In some instances, the barcoded sample nucleic acid fragments are enriched for one or more target sequences prior to further processing and sequencing. Because each fragment is coded as to its partition of origin, the sequence of that fragment may be attributed back to its origin based upon the presence of the barcode. This is schematically illustrated in FIG. 6. As shown in one example, a nucleic acid 604 originated from a first source 600 (e.g., individual chromosome, strand of nucleic acid, etc.) and a nucleic acid 606 derived from a different chromosome 602 or strand of nucleic acid are each partitioned along with their own sets of barcode oligonucleotides as described above.

Within each partition, each nucleic acid 604 and 606 is then processed to separately provide overlapping set of second fragments of the first fragment(s), e.g., second fragment sets 608 and 610. This processing also provides the second fragments with a barcode sequence that is the same for each of the second fragments derived from a particular first fragment. As shown, the barcode sequence for second fragment set 608 is denoted by “1” while the barcode sequence for fragment set 610 is denoted by “2.” A diverse library of barcodes may be used to differentially barcode large numbers of different fragment sets. However, it is not necessary for every second fragment set from a different first fragment to be barcoded with different barcode sequences. In some cases, multiple different first fragments may be processed concurrently to include the same barcode sequence. Diverse barcode libraries are described in detail elsewhere herein.

The barcoded fragments, e.g., from fragment sets 608 and 610, may then be pooled for sequencing using, for example, sequence by synthesis technologies available from Illumina or Ion Torrent. In some instances, the barcoded sample nucleic acid fragments are enriched (e.g., by nucleic acid capture or amplification) for one or more target sequences prior to further processing and sequencing the enriched, barcoded sample nucleic acid fragments, or derivatives thereof. Once sequenced, the sequence reads 612 can be attributed to their respective fragment set, e.g., as shown in aggregated reads 614 and 616, at least in part based upon the included barcodes, and in some cases, in part based upon the sequence of the fragment itself. The attributed sequence reads for each fragment set are then assembled to provide the assembled sequence for each sample fragment, e.g., sequences 618 and 620, which in turn, may be further attributed back to their respective original chromosomes (600 and 602). Methods and systems for assembling genomic sequences are described in, for example, U.S. Patent Publication No. US2015/0379196, filed Jun. 26, 2015, the full disclosure of which is hereby incorporated by reference in its entirety. In some examples, genomic sequences are assembled by de novo assembly and/or reference based assembly (e.g., mapping to a reference).

In some cases, sequencing the enriched set of barcoded nucleic acid molecules or derivatives thereof generates nucleic acid sequence information. The nucleic acid sequence information can be maternal or paternal nucleic acid sequence information comprising one or more nucleic acid sequences of a plurality of nucleic acid molecules derived from the maternal or paternal biological sample, respectively. The nucleic acid sequence information can be fetal nucleic acid sequence information comprising one or more fetal nucleic acid sequences of a plurality of fetal nucleic acid molecules derived from the maternal cell-free biological sample.

The nucleic acid sequence information can be processed to identify one or more haplotype blocks. The one or more haplotype blocks can be one or more maternal or paternal haplotype blocks. In some cases, the method further comprises processing nucleic acid sequence information from a maternal cell-free biological sample against the one or more maternal or paternal haplotype blocks to identify one or more genomic variations in one or more fetal nucleic acid sequences of the nucleic acid sequence information derived from the maternal cell-free biological sample.

In some cases, processing nucleic acid sequence information from a maternal cell-free biological sample against the one or more maternal or paternal haplotype blocks to identify one or more genomic variations in one or more fetal nucleic acid sequence comprises performing a relative haplotype dosing (RHDO) analysis. Methods of performing RHDO analysis to determine fetal genotype classification is described in, for example, New et al. “Noninvasive prenatal diagnosis of congenital adrenal hyperplasia using cell-free fetal DNA in maternal plasma” J Clin Endocrinol Metab. 2014 June; 99(6):E1022-30; Lo et al. “Maternal plasma DNA sequencing reveals the genome-wide genetic and mutational profile of the fetus Sci Transl Med. 2010 Dec. 8; 2(61):61ra91; Hui et al. “Universal haplotype-based noninvasive prenatal testing for single gene diseases.” Clinical Chemistry 2017 63:2. Published Dec. 8, 2016; and Lam et al. “Noninvasive prenatal diagnosis of monogenic diseases by targeted massively parallel sequencing of maternal plasma: Application to β-Thalassemia” Clin Chem. 2012 October; 58(10):1467-75, each of which is incorporated entirely herein by reference.

Relative haplotype dosing (RHDO) analysis can comprise performing a sequential probability ratio test (SPRT) of allelic imbalance in the nucleic acid sequence information derived from the maternal cell-free biological sample. SPRT can estimate the balance or imbalance of the dosage of a haplotype. Single nucleotide polymorphisms (SNPs) that are informative in an RHDO analysis can be SNPs that are heterozygous in the mother and homozygous in the father. SNPs that are informative in an RHDO analysis can be SNPs that are heterozygous in the mother and heterozygous in the father. In some cases, only informative SNPs are processed in the SPRT.

In some cases, paternal inheritance is determined by a Kolmogorov-Smirnov (KS) test. In some cases, SNPs that are informative in a KS test for paternal inheritance are SNPs that are heterozygous in the father and homozygous in the mother.

In some embodiments, digital relative mutation dosage (RMD) is used to determine fetal genotype classification. In some cases, RMD comprises the use of digital nucleic acid size selection (NASS). NASS can enrich for fetal DNA.

### Application of Methods and Systems to Phasing and Copy Number Assays

In one aspect of the systems and methods described herein, the ability to attribute sequence reads to longer originating molecules is used in determining phase information about the sequence. In one example, barcodes associated with sequences that reveal two or more specific gene variant sequences (e.g., alleles, genetic markers) are compared to determine whether or not that set of genetic markers reside on the same chromosome or different chromosomes in the sample. Such phasing information can be used in order to determine the relative copy number of certain target chromosomes or genes in a sample. An advantage of the described methods and symptoms is that multiple locations, loci, variants, etc. can be used to identify individual chromosomes or nucleic acid strands from which they originate in order to determine phasing and copy number information. Often, multiple locations (e.g., greater than 2, 3, 4, 5, 6, 7, 8, 9, 10, 20, 30 ,40, 50, 100, 500, 1000, 5000, 10000, 50000, 100000, or 500000) along a chromosome are used in order to determine phasing, haplotype and copy number variation information described herein.

By way of example, as noted above, the methods and systems described herein, by virtue of the partitioning and attribution aspects described above, can be useful at providing effective long sequence reads from individual nucleic acid fragments, e.g., individual nucleic acid molecules, despite utilizing sequencing technology that may provide relatively shorter sequence reads. Because these long sequence reads may be attributed to single starting fragments or molecules, variant locations in the sequence can, likewise, be attributed to a single molecule, and by extrapolation, to a single chromosome. In addition, one may employ the multiple locations on any given fragment, as alignment features for adjacent fragments, to provide aligned sequences that can be inferred as originating from the same chromosome. By way of example, a first fragment may be sequenced, and by virtue of the attribution methods and systems described above, the variants present on that sequence may all be attributed to a single chromosome. A second fragment that shares a plurality of these variants that are determined to be present only on one chromosome, may then be assumed to be derived from the same chromosome, and thus aligned with the first, to create a phased alignment of the two fragments. Repeating this allows for the identification of long range phase information. Identification of variants on a single chromosome can be obtained from either known references, e.g., HapMap, or from an aggregation of the sequencing data, e.g., showing differing variants on an otherwise identical sequence stretch. Targeting specific regions (e.g., whole exome) of the barcoded, short fragments allows for the retention of the phasing information generated by the above described methods while reducing the amount of sequencing required in the absence of targeting. Furthermore, because more information of interest can be captured in targeted phased libraries and because less input DNA is required compared to whole genome phased libraries, targeted libraries can be sequenced to a much greater depth (thereby increasing the accuracy of mutation calls, etc.) than whole genome phased libraries.

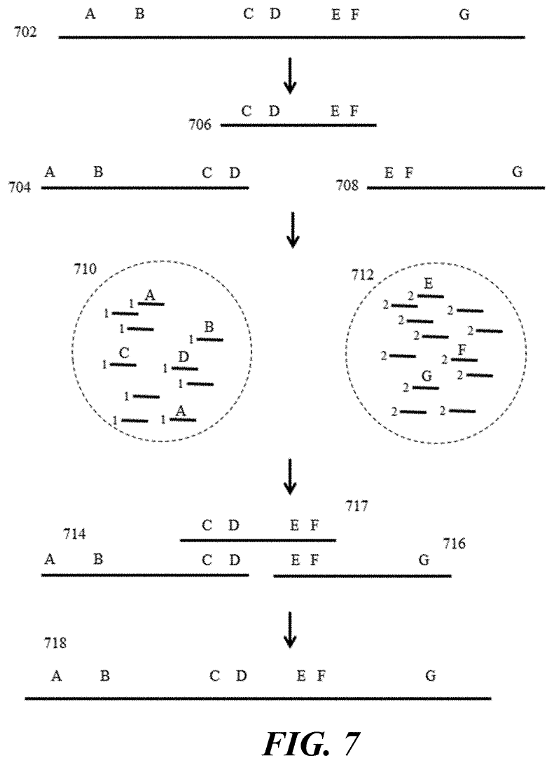

FIG. 7 provides a schematic illustration of an example phased sequencing process. As shown, an originating nucleic acid 702, such as, for example, a chromosome, a chromosome fragment, an exome, or other large, single nucleic acid molecule, can be fragmented into multiple large fragments 704, 706, 708. The originating nucleic acid 702 may include a number of sequence variants (A, B, C, D, E, F, and G) that are specific to the particular nucleic acid molecule, e.g., chromosome. In accordance with the processes described herein, the originating nucleic acid can be fragmented into multiple large, overlapping fragments 704, 706, and 708 that include subsets of the associated sequence variants. Each fragment can then be partitioned, further fragmented into subfragments, and barcoded, as described herein to provide multiple overlapping, barcoded subfragments of the larger fragments, where subfragments of a given larger fragment bear the same barcode sequence. For example, subfragments associated with barcode sequence “1” and barcode sequence “2” are shown in partitions 710 and 712, respectively. The barcoded subfragments can then be pooled, and subjected to enrichment (e.g., by nucleic acid capture) for particular sequences of interest (e.g., whole exome). The barcoded, enriched subfragments, or derivatives thereof, can then be sequenced, and the sequenced subfragments assembled to provide long fragment sequences 714, 716, and 717. One or more of the long fragment sequences 714, 716, and 717 can include multiple variants. The long fragment sequences may then be further assembled based upon overlapping phased variant information from sequences 714, 716, and 717 to provide a phased sequence 718, from which phased locations can be determined.

Once the phased locations are determined, one may further exploit that information in a variety of ways. For example, one can utilize knowledge of phased variants in assessing genetic risk for certain disorders, identify paternal vs. maternal characteristics, identify aneuploidies, or identify haplotyping information.

In some aspects of the systems and methods disclosed herein, copy number variation assays are performed using simultaneous detection of two or more phased genetic markers to improve the accuracy of copy number counting. Utilizing the phasing information can increase the relative strength of the signal compared to the variance under a naïve method just based on counting reads over multiple loci and across haplotypes. Additionally, utilizing phasing information allows for normalization of position-specific biases, boosting the signal substantially further. Copy number variation (CNV) accuracy may depend on myriad factors including sequencing depth, length of CNV, number of copies, etc). The methods and systems provided herein may determine CNV with an accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, the methods and systems provided herein determine CNV with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. Similarly, the methods and systems provided herein may detect phasing/haplotype information of two or more genetic variants with an accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, the methods and systems provided herein determine phasing or haplotype information with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. This disclosure also provides methods of removing locus-specific biases, where the locus-specific variance are reduced by at least 2-fold, 3-fold, 4-fold, 5-fold, 10-fold, 20-fold, 30-fold, 40-fold, 50-fold, 60-fold, 70-fold, 80-fold, 90-fold, 100-fold, 200-fold, 500-fold, 1000-fold, 5000-fold, or 10000-fold. The methods and systems provided herein can be used to detect variations in copy number, such as where the change in copy number reflects a change in the number of chromosomes, or portions of chromosomes. In some cases, the methods and systems provided herein can be used to detect variations in copy number of a gene present on the same chromosome.

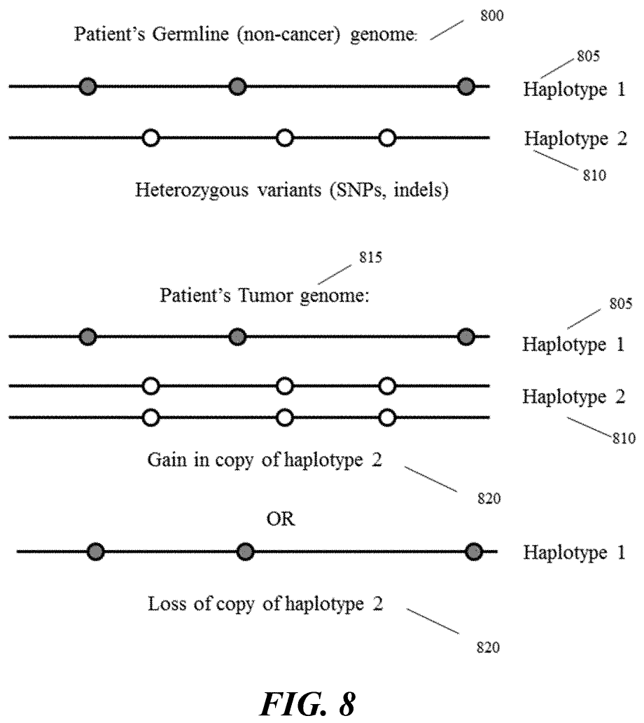

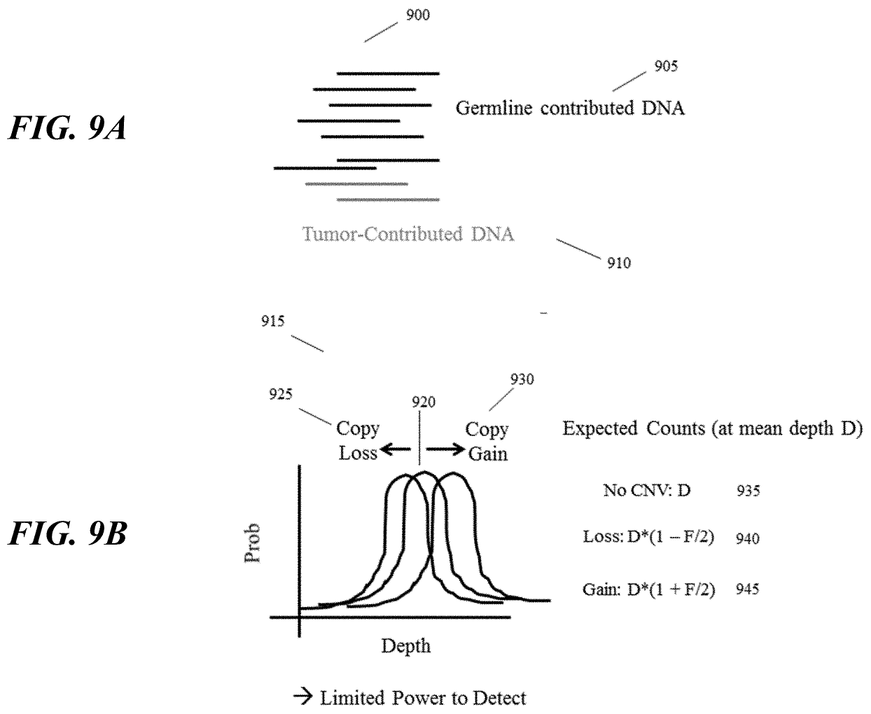

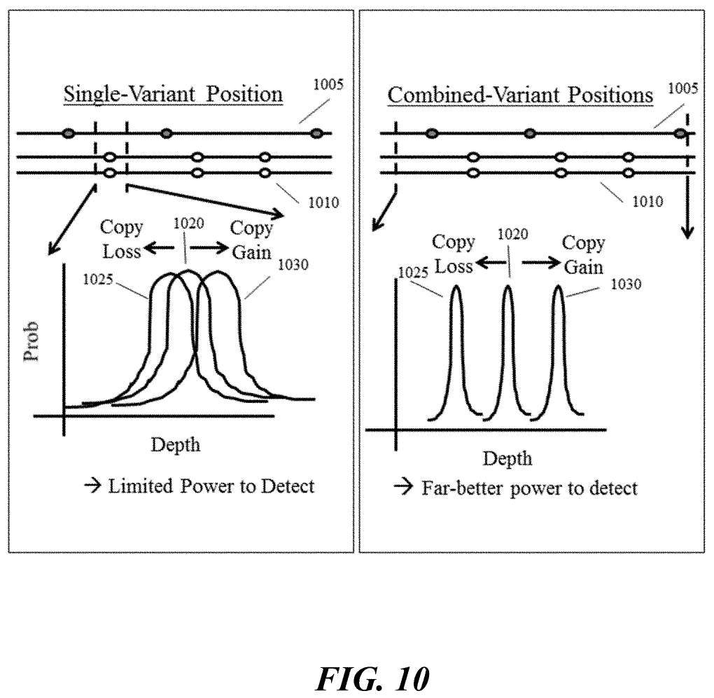

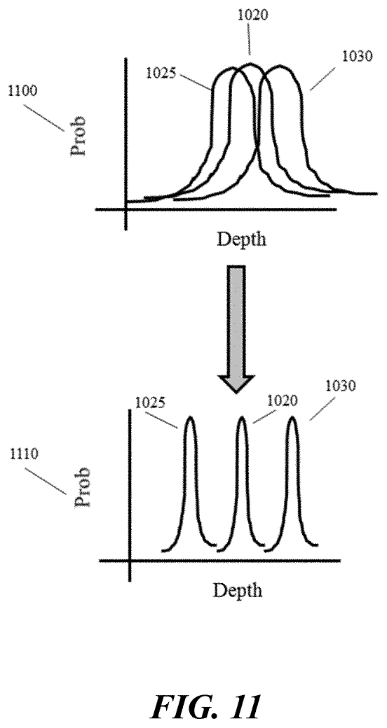

FIG. 8 (top panel) is a schematic illustrating a subset of a patient's germline (non-cancer) genome. This patient has a heterozygous genotype at the indicated loci and two separate haplotypes (1 and 2) 805, 810 located on separate chromosome strands. The patient's naturally-occurring variations (such as SNPs or indels) are depicted as circles. FIG. 8 also depicts the same patient's tumor genome 815. Certain cancers are associated with a gain in haplotype copy number. The middle panel depicts a gain in a haplotype 2, 810. Cancers may also be associated with a loss in haplotype number, as depicted in the bottom panel of FIG. 8, which shows a loss of haplotype 2 820. Common sequencing techniques cannot accurately determine this loss or gain of haplotype copies. As shown in FIG. 9A this is in part due to the fact that the tumor-contributed DNA 910 in a patient's blood is only a small fraction of the total DNA, of which a majority is the DNA contributed by normal tissue 905. This low concentration of tumor DNA results in imprecise detection of copy number with normal sequencing techniques, see FIG. 9B. The difference in the peaks of expected counts at mean depth D 935 for no copy variation 920 and the peaks for copy loss 925 (940) and copy gain 930 (945) is difficult to detect. For any given individual marker, the distribution of results of the copy number assay in replicate testing can be distributed around the correct answer in a manner approximating a Poisson distribution, where the width of the distribution is dependent on various sources of random error in the assay. Since for a give sample the change in copy number may be relatively small portion of the sample, broad probability distributions for monitoring of single genetic markers can mask the correct result. This difficulty is due to the fact that normal sequencing techniques only look at one single variant position of a haplotype at a time, as shown in FIG. 10 (left panel). Using such techniques, there can be significant overlap between peaks representing copy loss 1025, normal copy 1020, and copy gain 1030. The targeted techniques disclosed herein allow for detection of whole (or partial) haplotypes, increasing the resolution and improving the detection of copy gain and loss, FIG. 10 (right panel). This improvement is schematically shown in FIG. 11, where normal detection 1100 results in spread out, overlapping peaks while the techniques herein 1110 allow for finer peaks and improved resolution of copy gain or loss. The use of simultaneous monitoring of two or more phased genetic markers, particularly markers that are known to be co-located on a single chromosome, and which can therefore most likely always appear in greater or lesser number in a synchronized, non-random fashion has the effect of narrowing the width of the expected results distribution and simultaneously improving the accuracy of the count.

In addition to advantages in detecting and diagnosing cancers, the methods and systems provided herein also provide more accurate and sensitive processes for detecting fetal aneuploidy.

Fetal aneuploidies are aberrations in fetal chromosome number. Aneuploidies commonly result in significant physical and neurological impairments. For example, a reduction in the number of X chromosomes is responsible for Turner's syndrome. An increase in copy number of chromosome number 21 results in Down Syndrome. Invasive testing such as amniocentesis or Chorionic Villus Sampling (CVS) can lead to risk of pregnancy loss and less invasive methods of testing the maternal blood are used here.

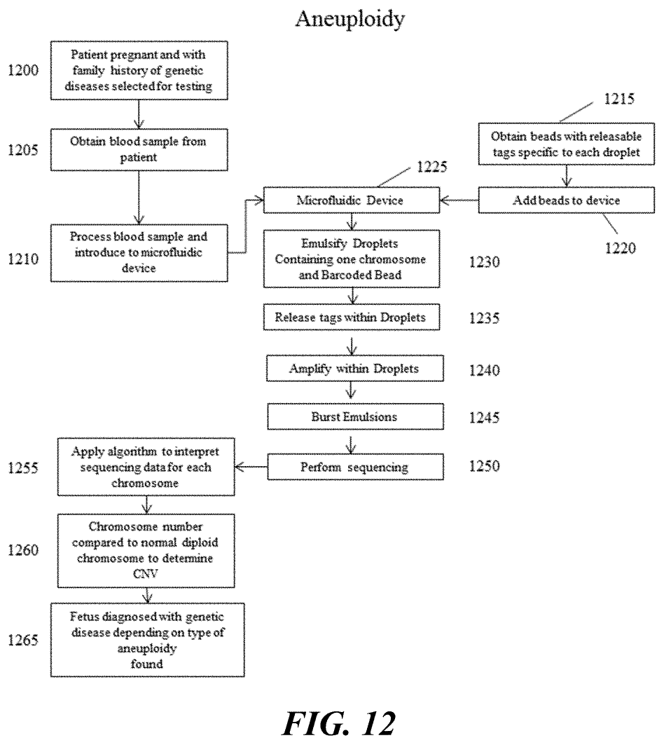

Methods described herein may be useful in non-invasively detecting fetal aneuploidies. An example process is shown in FIG. 12. A pregnant woman at risk of carrying a fetus with an aneuploid genome is tested, 1200. A maternal blood sample containing fetal genetic material is collected, 1205. Genetic material (e.g., cell-free nucleic acids) is then extracted from the blood sample, 1210. A set of barcoded beads may also be obtained, 1215. The beads can be linked to oligonucleotides containing one or more barcode sequences, as well as a primer, such as a random N-mer or other targeted primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, a sample, 1210, barcoded beads, 1220, and, in some cases, other reagents, e.g., a reducing agent, are combined and subjected to partitioning. By way of example, such partitioning may involve introducing the components to a droplet generation system, such as a microfluidic device, 1225. With the aid of the microfluidic device 1225, a water-in-oil emulsion 1230 may be formed, where the emulsion contains aqueous droplets that contain sample nucleic acid, 1210, barcoded beads, 1215, and, in some cases, a reducing agent. The reducing agent may dissolve or degrade the barcoded beads, thereby releasing the oligonucleotides with the barcodes and random N-mers from the beads within the droplets, 1235. The random N-mers may then prime different regions of the sample nucleic acid, resulting in amplified copies of the sample after amplification, where each copy is tagged with a barcode sequence, 1240. In some cases, each droplet contains a set of oligonucleotides that contain identical barcode sequences and different random N-mer sequences. In other cases, each droplet contains a set of oligonucleotides that contain identical barcode sequences and one or more primer sequence(s) directed to a specific target(s) of interest (e.g., a specific gene, locus, or whole exome). In other embodiments, individual droplets comprise unique barcode sequences; or, in some cases, a certain proportion of the total population of droplets has unique sequences. Subsequently, the emulsion is broken, 1245 and the barcoded sample nucleic acid fragments can be enriched for particular targets of interest. For example, barcoded sample fragments can be targeted by nucleic acid capture (e.g., hybridization to capture probes) to enrich for sequences of interest (e.g., the whole exome). In other cases, barcoded sample nucleic acid fragments can be enriched by nucleic acid amplification using primers directed to sequences of interest. Subsequent (or prior to enrichment), additional sequences (e.g., sequences that aid in particular sequencing methods, additional barcodes, etc.) may be added, via, for example, amplification methods (e.g., PCR). Sequencing may then be performed via any suitable type of sequencing platform (e.g., Illumina, Ion Torrent, Pacific Biosciences SMRT, Roche 454 sequencing SOLiD sequencing, etc.), 1250, and an algorithm applied to interpret the sequencing data, 1255. Sequencing algorithms are generally capable, for example, of performing analysis of barcodes to align sequencing reads and/or identify the sample from which a particular sequence read belongs. The aligned sequences may be further attributed to their respective genetic origins (e.g., chromosomes) based upon, the unique barcodes attached. The number of chromosome copies is then compared to that of a normal diploid chromosome, 1260. The patient is informed of any copy number aberrations for different chromosomes and the associated risks/disease, 1265.

Phasing, e.g. determining whether genetic variants are linked or reside on different chromosomes can provide useful information for a variety of applications. By way of example, phasing is useful for determining if certain translocations of a genome associated with diseases are present. Detection of such translocations can also allow for differential diagnosis and modified treatment. Determination of which alleles in a genome are linked can be useful for considering how genes are inherited.

It can often be useful to know the pattern of alleles, the haplotype, for each individual chromosome of a chromosome pair. For example, two copies of an inactivating mutation present on one chromosome may be of limited consequence, but may have significant effect if distributed between the two chromosomes, e.g., where neither chromosome supplies active gene product. These effects can be expressed e.g., with increased risk of disease or lack of response to certain medications.

### Application of Methods and Systems to Characterization of Structural Variations

In other applications, the method and systems described herein are highly useful in obtaining the long range molecular sequence information for identification and characterization of a wide range of different genetic structural variations. As noted above, these variations include a wide variety of different variant events, including insertions, deletions, duplications, retrotransposons, translocations, inversions short and long tandem repeats, and the like. These structural variations are of significant scientific interest, as they are believed to be associated with a range of diverse genetic diseases. In some cases, the disclosure provides methods and systems useful in obtaining targeted, long range molecule sequence information for identification and characterization of different genetic structural variations from a maternal cell-free biological sample.

Despite the interest in these variations, there are few effective and efficient methods of identifying and characterizing these structural variations. In part, this is because these variations are not characterized by the presence of abnormal sequence segments, but instead, involve and abnormal sequence context of what would be considered normal sequence segments, or simply missing sequence information. Because of their relatively short read lengths, most sequencing technologies are unable to provide significant context, and especially, long range sequence context, e.g., beyond their read lengths, for the sequence reads they produce, and thus lose the identification of these variations in the assembly process. The difficulties in identifying these variations is further complicated by the ensemble approach of these technologies in which many molecules, e.g., multiple chromosomes, are combined to yield a consensus sequence that may include genomic material that both includes and does not include the variation.

In the context of the presently described methods and systems, however, one can utilize short read sequencing technologies to derive long range sequence information that is attributable to individual originating nucleic acid molecules, and thus retain the long range sequence context of variant regions contained in whole or in part in those individual molecules. By targeting specific regions (e.g., whole exome) of the barcoded, short fragments allows for the retention of the long range phasing information while reducing the amount of sequencing required in the absence of targeting. Furthermore, because more information of interest can be captured in targeted phased libraries and because less input DNA is required compared to whole genome phased libraries, targeted libraries can be sequenced to a much greater depth (thereby increasing the accuracy of mutation calls, etc.) than whole genome phased libraries.

As described above, the methods and systems described herein are capable of providing long range sequence information that is attributable to individual originating nucleic acid molecules, and further, in possessing this long range sequence information, inferring even longer range sequence context, through the comparing and overlapping of these longer sequence information. Such long range sequence information and/or inferred sequence context allows the identification and characterization numerous structural variations not easily identified using available techniques.

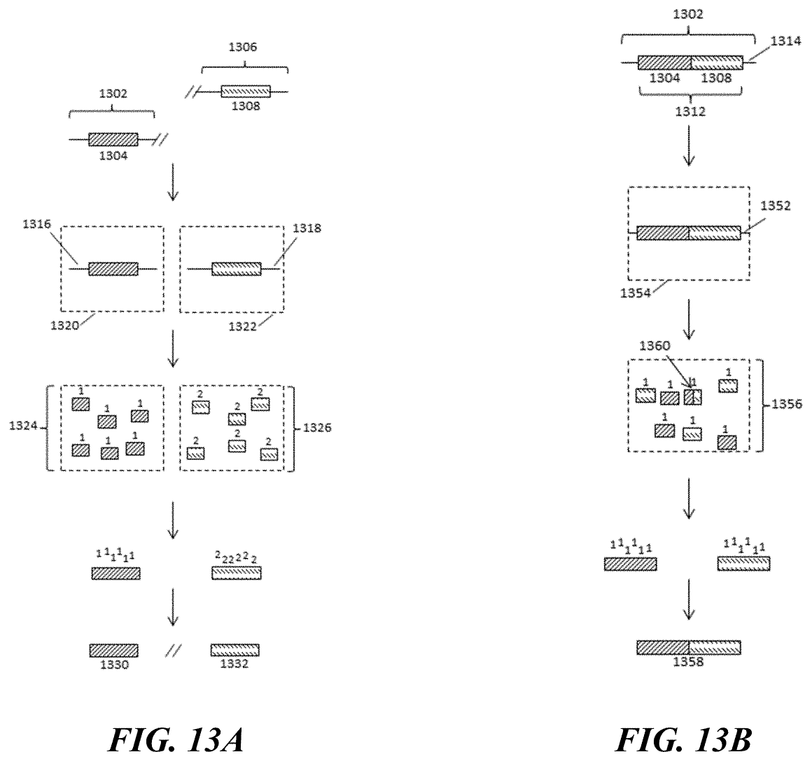

While illustrated in simplified fashion in FIG. 2, FIGS. 13A and 13B provide a more detailed example process for identifying certain types of structural variations using the methods and systems described herein. As shown, the genome of an organism, or tissue from an organism, might ordinarily include the first genotype illustrated in FIG. 13A, where a first gene region 1302 including first gene 1304 is separated from a second gene region 1306 including second gene 1308. This separation may reflect a range of distances between the genes, including, e.g., different regions in the same exon, different exons on the same chromosome, different chromosomes, etc. As shown in FIG. 13B however, a genotype is shown that reflects a translocation event having occurred in which gene 1308 is inserted into gene region 1304 such that it creates a gene fusion between genes 1304 and 1308 as gene fusion 1312 in variant sequence 1314.

Current methods for detecting large genomic structural variants (such as large inversions or translocations) rely on read pairs that span the breakpoints of the variants (for example the genomic loci where the translocated parts fused together). To ensure that such read pairs are observed during a sequencing experiment, very deep sequencing can be required. In traditional targeted sequencing (such as exome sequencing) in the absence of phasing information, detecting structural variants with current sequencing technologies is almost impossible, unless the breakpoint is within the targeted regions (e.g. in an exon), which is very unlikely.

Information provided by the barcode methods and systems described herein, however, can greatly improve the ability to detect structural variants. Intuitively, the loci to the left and to the right of a breakpoint can tend to be on a common fragment of genomic DNA and therefore be maintained within a single partition, and thus barcoded with a common or shared barcode sequence. Due to the stochastic nature of shearing, this sharing of barcodes decreases as the sequences are more distant from the breakpoint. Using statistical methods, one can determine whether the barcode overlap between two genomic loci is significantly larger than what would be expected by chance. Such an overlap may suggest the presence of a breakpoint. Importantly, the barcode information complements information provided by traditional sequencing (such as information from reads spanning the breakpoint) if such information is available. Targeting specific regions (e.g., whole exome) of the barcoded, short fragments allows for the retention of the phasing information and of the fusion events while reducing the amount of sequencing required in the absence of targeting. Furthermore, because more information of interest can be captured in targeted phased libraries and because less input DNA is required compared to whole genome phased libraries, targeted libraries can be sequenced to a much greater depth (thereby increasing the accuracy of mutation calls, detection of the fusion events, etc.) than whole genome phased libraries.

In the context of the methods described herein, the genomic material from the organism, including the relevant gene regions is fragmented such that it includes relatively long fragments, as described above. This is illustrated with respect to the non-translocated genotype in FIG. 13A. As shown two long individual first molecule fragments 1316 and 1318 are created that include gene regions 1302 and 1306 respectively. These fragments are separately partitioned into partitions 1320 and 1322, respectively, and each of the first fragments is fragmented into a number of second fragments 1324 and 1326, respectively within the partition, which fragmenting process attaches a unique identifier tag or barcode sequence to the second fragments that is common to all of the second fragments within a given partition. The tag or barcode is indicated by “1” or “2,” for each of partitions 1320 and 1322, respectively. As a result, completely separate genes 1304 and 1308 can result in differently partitioned, and differently barcoded groups of second fragments.

Once barcoded, the second fragments may then be pooled, targeted, and subjected to nucleic acid sequencing processes, which can provide both the sequence of the second fragment as well as the barcode sequence for that fragment. Based upon the presence of a particular barcode, e.g., 1 or 2, a the second fragment sequences may then be attributed to a certain originating sequence, e.g., gene 1304 or 1308, as shown by the attribution of barcodes to each sequence. In some cases, mapping of barcoded second fragment sequences as to separate originating first fragment sequences may be sufficiently definitive to determine that no translocation has occurred. However, in some cases, one may assemble the second fragment sequences to provide an assembled sequence for all or a portion of the originating first fragment sequence, e.g., as shown by assembled sequences 1330 and 1332.

In contrast to the non-translocated genotype example shown in FIG. 13A, FIG. 13B shows a schematic illustration of the same process applied to a translocation containing genotype. As shown, a first long nucleic acid fragment 1352 is generated from the variant sequence 1314, and includes at least a portion of the translocation variant, e.g., gene fusion 1312. The first fragment 1352 is then partitioned into discrete partition 1354. Within partition 1354, first fragment 1352 is further fragmented into second fragments 1356 that again, include unique barcodes that are the same for all second fragments 1356 within the partition 1354 (shown as barcode “1”). As above, pooling the second fragments and sequencing provides the underlying sequences of the second fragments as well as their associated barcodes. These barcoded sequences can then be attributed to their respective gene sequences. As shown, however, both genes can reflect attributed second fragment sequences that include the same barcode sequences, indicating that they originated from the same partition, and potentially the same originating molecule, indicating a gene fusion. This may be further validated by providing a number of overlapping first fragments that also include at least portions of the gene fusion, but processed in different partitions with different barcodes.

In some cases, the presence of multiple different barcode sequences (and their underlying fragment sequences) that attribute to each of the originally separated genes can be indicative of the presence of a gene fusion or other translocation event. In some cases, attribution of at least 2 barcodes, at least 3 different barcodes, at least 4 different barcodes, at least 5 different barcodes, at least 10 different barcodes, at least 20 different barcodes or more, to two genetic regions that would have been expected to have been separated based upon a reference sequence, may provide indication of a translocation event that has placed those regions proximal to, adjacent to or otherwise integrated with each other. In some cases, the size of the fragments that are partitioned can indicate the sensitivity with which one can identify variant linkage. In particular, where the fragments in a given droplet are 10 kb in length, it would be expected that linkages that are within that 10 kb size range would be detectable.

Likewise, where both the variant and the wild type structure fall within the same 10 kb fragment, it would be expected that identification of that variant may be more difficult, as both would show linkage through common or shared barcodes. As such, fragment size selection may be used to adjust the relative proximity of detected linked sequences, whether as wild type or variants. In general, however, structural variants that result in proximal sequences that are normally separated by more than 100 bases, more than 500 bases, more than 1 kb, 10 kb, more than 20 kb, more than 30 kb, more than 40 kb, more than 50 kb, more than 60 kb, more than 70 kb, more than 80 kb, more than 90 kb, more than 100 kb, more than 200 kb or even greater, may be readily identified herein by identifying the linkage between those unlinked sequence segments in variant genomes, which linkage is indicated by shared or common barcodes, and/or, as noted, by sequence data that spans a breakpoint. Such linkage is generally identifiable when those linked sequences are separated within the genomic sequence by less than 50 kb, less than 40 kb, less than 30 kb, less than 20 kb, less than 10 kb, less than 5 kb, less than 4 kb, less than 3 kb, less than 2 kb, less than 1 kb, less than 500 bases, less than 200 bases or even less.

In some cases, a structural variation resulting in two sequences being positioned proximal to each other or linked, where they would normally be separated by, e.g., more than 10 kb, more than 20 kb, more than 30 kb, more than 40 kb, or more than 50 kb or more, may be identified by the percentage of the total number of mappable barcoded sequences that include barcodes that are common to the two sequence portions.

As will be appreciated, in some cases, the processes described herein can ensure that sequences that are within a certain sequence distance will be included, whether as wild type or variant sequences, within a single partition, e.g., as a single nucleic acid fragment. For example, where common or overlapping barcode sequences are greater than 1% of the total number of barcodes mapped to the two sequences, it may be used to identify linkage as between two sequence segments, and particularly, as between two sequence segments that would not normally be linked, e.g., a structural variation. In some cases, the shared or common barcodes can be more than 2%, more than 3%, more than 4%, more than 5%, more than 6%, more than 7%, more than 8%, and in some cases more than 9% or even more than 10% of the total mappable barcodes to two normally separated sequences, in order to identify a structural linkage that constitutes a structural variation within the genome. In some cases, the shared or common barcodes can be detected at a proportion or number that is statistically significantly greater than a control genome that is known not to have the structural variation. Additionally, where second sequence fragments span the point where the variant sequence meets the “normal” sequence, or “breakpoint,” e.g., as in second fragment 1358 one can use this information as additional evidence of the gene fusion.

Again, as above, one can further elucidate the structure of the gene fusion 1312, by assembling the second fragment sequences to yield the assembled sequence of the gene fusion 1312, shown as assembled sequence 1360.

Further, while the presence of the barcode sequences allows the assembly of the short sequences into sequences for the longer originating fragments, these longer fragments also permit the inference of longer range sequence information from overlapping long fragments assembled from different, overlapping originating long fragments. This resulting assembly allows for longer range sequence level identification and characterization of gene fusion 1312.

In some cases, the methods described above are useful in identifying the presence of retrotransposons. Retrotransposons can be created by transcription followed by reverse transcription of spliced messenger RNA (mRNA) and insertion into a new location in the genome. Hence, these structural variants lack introns and are often interchromosomal but otherwise have diverse features. When retrotransposons introduce functional copies of genes they are referred to as retrogenes, which have been reported in human and Drosophila genomes. In other cases, retrocopies may contain the entire transcript, specific transcript isoforms or an incomplete transcript. In addition, alternative transcription start sites and promoter sequences sometimes reside within a transcript so retrotransposons sometimes introduce promotor sequences within the reinserted region of the genome that may drive expression of downstream sequences.

Unlike tandem duplications, retrotransposons insert far away from the parental gene within exons or introns. When inserted near genes retrotransposons can exploit local regulatory sequences for expression. Insertions near genes can also inactivate the receiving gene or create new chimera transcripts. Retrotransposon mediated chimeric gene transcripts have been reported in RNA-seq data from human samples.

Despite the significance of retrotransposons their detection can be limited to directed approaches relying on paired read support from mate pair libraries, exon-exon junction discovery in whole genome sequencing (WGS) or RNA-seq recognition of retrotransposon chimeras. All of these methods can have false positives that complicate analysis.

Retrotransposons can be identified from whole genome libraries using the systems and methods described herein, and their insertion site can be mapped using the barcode mapping discussed above. For example, the Ceph NA12878 genome has a SKA3-DDX10 chimeric retrotransposon. The SKA3 intron-less transcript is inserted in between exons 10 and 11 of DDX10. Furthermore the CBX3-C15ORF17 retrotransposon can also be detected in NA12878 using the methods described herein. Isoform 2 of CBX3 is inserted in between exons 2 and 3 of C15ORF17. This chimeric transcript has been observed in 20% of European RNA-seq samples from the HapMap project (D. R. Schrider et al. PLoS Genetics 2013).

Retrotransposons can also be detected in whole exome libraries prepared using the methods and systems described herein. While retrotransposons are easily enriched with exome targeting it can be difficult or not possible to differentiate between a translocation event and a retrotransposon since introns are removed during capture. However, using the systems and methods described herein, one may identify retrotransposons in whole exome sequencing (WES) libraries by introducing intronic baits for suspected retrotransposons (see also U.S. Patent Publication No. US2016/0122817, filed Oct. 29, 2015, incorporated herein by reference in its entirety for all purposes) or by enriching (e.g., by nucleic acid sequence capture) for regions containing suspected retrotransposons in the barcoded short fragments. Alternatively, one may utilize barcoded oligonucleotides comprising a sequence targeted to suspected retrotransposons to barcode these regions directly in partitions. Lack of intron signal can be indicative of retrotransposon structural variants whereas intron signal can be indicative of a translocation. As will be appreciated, the ability to use longer range sequence context in identifying and characterizing of the above-described variations is equally applicable to identifying the range of other structural variations, including insertions, deletion, retrotransposons, inversions, etc., by mapping barcodes to regions within the variation, and/or spanning the variation.

Diseases & Disorders Arising from Copy Number Variation

The present methods and systems provide a highly accurate and sensitive approach to diagnosing and/or detecting a wide range of diseases and disorders. Diseases associated with copy number variations can include, for example, DiGeorge/velocardiofacial syndrome (22q11.2 deletion), Prader-Willi syndrome (15q11-q13 deletion), Williams-Beuren syndrome (7q11.23 deletion), Miller-Dieker syndrome (MDLS) (17p13.3 microdeletion), Smith-Magenis syndrome (SMS) (17p11.2 microdeletion), Neurofibromatosis Type 1 (NF1) (17q11.2 microdeletion), Phelan-McErmid Syndrome (22q13 deletion), Rett syndrome (loss-of-function mutations in MECp2 on chromosome Xq28), Merzbacher disease (CNV of PLP1), spinal muscular atrophy (SMA) (homozygous absence of telomerec SMN1 on chromosome 5q13), Potocki-Lupski Syndrome (PTLS, duplication of chromosome 17p.11.2). Additional copies of the PMP22 gene can be associated with Charcot-Marie-Tooth neuropathy type IA (CMT1A) and hereditary neuropathy with liability to pressure palsies (HNPP). The disease can be a disease described in Lupski J. (2007) Nature Genetics 39: S43-S47.

The methods and systems provided herein can also accurately detect or diagnose a wide range of fetal aneuploidies. Often, the methods provided herein comprise analyzing a sample (e.g., blood sample) taken from a pregnant woman in order to evaluate the fetal nucleic acids within the sample. Fetal aneuploidies, can include, e.g., trisomy 13 (Patau syndrome), trisomy 18 (Edwards syndrome), trisomy 21 (Down Syndrome), Klinefelter Syndrome (XXY), monosomy of one or more chromosomes (X chromosome monosomy, Turner's syndrome), trisomy X, trisomy of one or more chromosomes, tetrasomy or pentasomy of one or more chromosomes (e.g., XXXX, XXYY, XXXY, XYYY, XXXXX, XXXXY, XXXYY, XYYYY and XXYYY), triploidy (three of every chromosome, e.g. 69 chromosomes in humans), tetraploidy (four of every chromosome, e.g. 92 chromosomes in humans), and multiploidy. In some embodiments, an aneuploidy can be a segmental aneuploidy. Segmental aneuploidies can include, e.g., 1p36 duplication, dup(17)(p11.2p11.2) syndrome, Down syndrome, Pelizaeus-Merzbacher disease, dup(22)(q11.2q11.2) syndrome, and cat-eye syndrome. In some cases, an abnormal genotype, e.g., fetal genotype, is due to one or more deletions of sex or autosomal chromosomes, which can result in a condition such as Cri-du-chat syndrome, Wolf-Hirschhorn, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, Steroid sulfatase deficiency, Kallmann syndrome, Microphthalmia with linear skin defects, Adrenal hypoplasia, Glycerol kinase deficiency, Pelizaeus-Merzbacher disease, Testis-determining factor on Y, Azospermia (factor a), Azospermia (factor b), Azospermia (factor c), or 1p36 deletion. In some embodiments, a decrease in chromosomal number results in an XO syndrome.

Excessive genomic DNA copy number variation is also associated with Li-Fraumeni cancer predisposition syndrome (Shlien et al. (2008) PNAS 105:11264-9). CNV is associated with malformation syndromes, including CHARGE (coloboma, heart anomaly, choanal atresia, retardation, genital, and ear anomalies), Peters-Plus, Pitt-Hopkins, and thrombocytopenia-absent radius syndrome (see e.g., Ropers H H (2007) Am J of Hum Genetics 81: 199-207). The relationship between copy number variations and cancer is described, e.g., in Shlien A. and Malkin D. (2009) Genome Med. 1(6): 62. Copy number variations are associated with, e.g., autism, schizophrenia, and idiopathic learning disability. See e.g., Sebat J., et al. (2007) Science 316: 445-9; Pinto J. et al.

As described herein, the methods and systems provided herein are also useful to detect CNVs associated with different types of cancer. For example, the methods and systems can be used to detect EGFR copy number, which can be increased in non-small cell lung cancer.

The methods and systems provided herein can also be used to determine a subject's level of susceptibility to a particular disease or disorder, including susceptibility to infection from a pathogen (e.g., viral, bacterial, microbial, fungal, etc.). For example, the methods can be used to determine a subject's susceptibility to HIV infection by analyzing the copy number of CCL3L1, given that a relatively high level of CCL3L1 is associated with lower susceptibility to HIV infection (Gonzalez E. et al. (2005) Science 307: 1434-1440). In another example, the methods can be used to determine a subject's susceptibility to system lupus erythematosus. In such cases, for example, the methods can be used to detect copy number of FCGR3B (CD16 cell surface immunoglobulin receptor) since a low copy number of this molecule is associated with an increased susceptibility to systemic lupus erythematosus (Aitman T. J. et al. (2006) Nature 439: 851-855). The methods and systems provided herein can also be used to detect CNVs associated with other diseases or disorders, such as CNVs associated with autism, schizophrenia, or idiopathic learning disability (Kinght et al., (1999) The Lancet 354 (9191): 1676-81.). Similarly, the methods and systems can be used to detect autosomal-dominant microtia, which is linked to five tandem copies of a copy-number-variable region at chromosome 4p16 (Balikova I. (2008) Am J. Hum Genet. 82: 181-187).

### Detection, Diagnosis and Treatment of Diseases and Disorders

The methods and systems provided herein can also assist with the detection, diagnosis, and treatment of a disease or disorder. In some cases, a method comprises detecting a disease or disorder using a system or method described herein and further providing a treatment to a subject based on the detection of the disease. For example, if a cancer is detected, the subject may be treated by a surgical intervention, by administering a drug designed to treat such cancer, by providing a hormonal therapy, and/or by administering radiation or more generalized chemotherapy.

Often, the methods and systems also permit a differential diagnosis and may further comprise treating a patient with a targeted therapy. In general, differential diagnosis of a disease or disorder (or absence thereof) can be achieved by determining and characterizing a sequence of a sample nucleic acid obtained from a subject suspected of having the disease or disorder and further characterizing the sample nucleic acid as indicative of a disorder or disease state (or absence thereof) by comparing it to a sequence and/or sequence characterization of a reference nucleic acid indicative of the presence (or absence) of the disorder or disease state.

The reference nucleic acid sequence may be derived from a genome that is indicative of an absence of a disease or disorder state (e.g., germline nucleic acid) or may be derived from a genome that is indicative of a disease or disorder state (e.g., cancer nucleic acid, nucleic acid indicative of an aneuploidy, etc.). Moreover, the reference nucleic acid sequence (e.g., having lengths of longer than 1 kb, longer than 5 kb, longer than 10 kb, longer than 15 kb, longer than 20 kb, longer than 30 kb, longer than 40 kb, longer than 50 kb, longer than 60 kb, longer than 70 kb, longer than 80 kb, longer than 90 kb or even longer than 100 kb) may be characterized in one or more respects, with non-limiting examples that include determining the presence (or absence) of a particular sequence, determining the presence (or absence) of a particular haplotype, determining the presence (or absence) of one or more genetic variations (e.g., structural variations (e.g., a copy number variation, an insertion, a deletion, a translocation, an inversion, a retrotransposon, a rearrangement, a repeat expansion, a duplication, etc.), single nucleotide polymorphisms (SNPs), etc.) and combinations thereof. Moreover, any suitable type and number of sequence characteristics of the reference sequence can be used to characterize the sequence of the sample nucleic acid. For example, one or more genetic variations (or lack thereof) or structural variations (or lack thereof) of a reference nucleic acid sequence may be used as a sequence signature to identify the reference nucleic acid as indicative of the presence (or absence) of a disorder or disease state. Based on the characterization of the reference nucleic acid sequence utilized, the sample nucleic acid sequence can be characterized in a similar manner and further characterized/identified as derived (or not derived) from a nucleic acid indicative of the disorder or disease based upon whether or not it displays a similar character to the reference nucleic acid sequence. In some cases, characterizations of sample nucleic acid sequence and/or the reference nucleic acid sequence and their comparisons may be completed with the aid of a programmed computer processor. In some cases, such a programmed computer processor can be included in a computer control system, such as in an example computer control system described elsewhere herein.

The sample nucleic acid may be obtained from any suitable source, including sample sources and biological sample sources described elsewhere herein. In some cases, the sample nucleic acid may comprise cell-free nucleic acid. In some cases, the sample nucleic acid may comprise fetal nucleic acid. In some cases, the sample nucleic acid may comprise circulating maternal DNA. Circulating maternal and/or fetal nucleic acid may be derived or obtained from, for example, from a subject's blood, plasma, other bodily fluid or tissue.

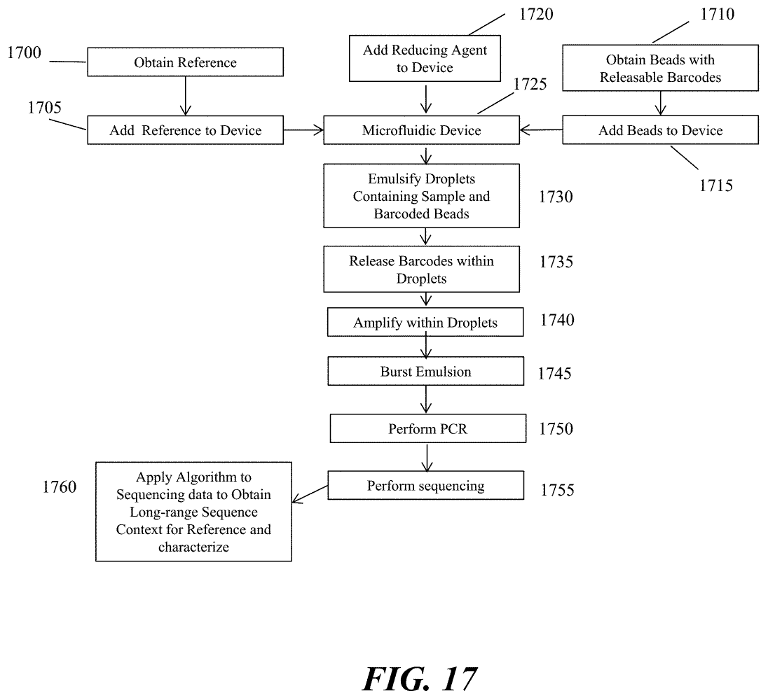

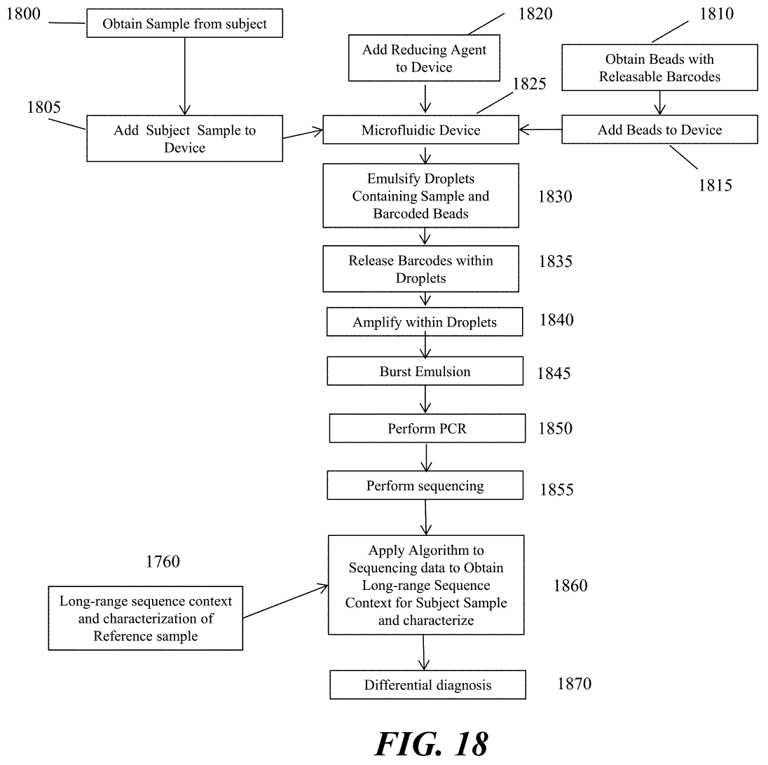

FIGS. 17-18 illustrate an example method for characterizing a sample nucleic acid in the context of disease detection and diagnosis. FIG. 17 demonstrates an example method by which long range sequence context can be determined for a reference nucleic acid (e.g., germline nucleic acid (e.g., germline genomic DNA), nucleic acid associated with a particular disorder or disease state) from shorter barcoded fragments, such as, for example in a manner analogous to that shown in FIG. 6. With respect to FIG. 17, a reference nucleic acid may be obtained 1700, and a set of barcoded beads may also be obtained, 1710. The beads can be linked to oligonucleotides containing one or more barcode sequences, as well as a primer, such as a random N-mer or other targeted primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, reference nucleic acid, 1705, barcoded beads, 1715, and, in some cases, other reagents, e.g., a reducing agent, 1720, are combined and subject to partitioning. In some cases, the reference nucleic acid 1700 may be fragmented prior to partitioning and at least some of the resulting fragments are partitioned as 1705 for barcoding. By way of example, such partitioning may involve introducing the components to a droplet generation system, such as a microfluidic device, 1725. With the aid of the microfluidic device 1725, a water-in-oil emulsion 1730 may be formed, where the emulsion contains aqueous droplets that contain reference nucleic acid, 1705, reducing agent, 1720, and barcoded beads, 1715. The reducing agent may dissolve or degrade the barcoded beads, thereby releasing the oligonucleotides with the barcodes and random N-mers from the beads within the droplets, 1735. The random N-mers may then prime different regions of the reference nucleic acid, resulting in amplified copies of the reference nucleic acid after amplification, where each copy is tagged with a barcode sequence, 1740. In some cases, amplification 1740 may be achieved by a method analogous to that described elsewhere herein and schematically depicted in FIG. 5. In some cases, each droplet contains a set of oligonucleotides that contain identical barcode sequences and different random N-mer sequences. In other cases, each droplet contains a set of oligonucleotides that contain identical barcode sequences and one or more primer sequences directed against one or more target regions. Subsequently, the emulsion is broken, 1745 and the barcoded sample nucleic acid fragments can be enriched for particular targets of interest. For example, barcoded sample fragments can be targeted by nucleic acid capture (e.g., hybridization to capture probes) to enrich for sequences of interest (e.g., the whole exome). In other cases, barcoded sample nucleic acid fragments can be enriched by nucleic acid amplification using primers directed to sequences of interest. Subsequent (or prior to enrichment), additional sequences (e.g., sequences that aid in particular sequencing methods, additional barcodes, etc.) may be added, via, for example, amplification methods, 1750 (e.g., PCR). Sequencing may then be performed, 1755, and an algorithm applied to interpret the sequencing data, 1760. In some cases, interpretation of the sequencing data 1760 may include providing a sequence for at least a portion of the reference nucleic acid. In some cases, long range sequence context for the reference nucleic acid is obtained and characterized such as, for example, in the case where the reference nucleic acid is derived from a disease state (e.g., determination of one or more haplotypes as described elsewhere herein, determination of one or more structural variations (e.g., a copy number variation, an insertion, a deletion, a translocation, an inversion, a rearrangement, a repeat expansion, a duplication, retrotransposon, a gene fusion, etc.), calling of one or more SNPs, etc.). In some cases, variants can be called for various reference nucleic acids obtained from a source and inferred contigs generated to provide longer range sequence context, such as is described elsewhere herein with respect to FIG. 7.

FIG. 18 demonstrates an example of characterizing a sample nucleic acid sequence from the reference 1760 characterization obtained as shown in FIG. 17. Long range sequence context can be obtained for the sample nucleic acid from sequencing of shorter barcoded fragments as is described elsewhere herein, such as, for example, via the method schematically depicted in FIG. 6. As shown in FIG. 18, a nucleic acid sample (e.g., a sample comprising a cell free nucleic acid) can be obtained from a subject suspected of having a disorder or disease 2100 and a set of barcoded beads may also be obtained, 1810. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, sample nucleic acid, 1805, barcoded beads, 1815, and, in some cases, other reagents, e.g., a reducing agent, 1820, are combined and subject to partitioning. In some cases, the fetal sample 1800 is fragmented prior to partitioning and at least some of the resulting fragments are partitioned as 1805 for barcoding. By way of example, such partitioning may involve introducing the components to a droplet generation system, such as a microfluidic device, 1825. With the aid of the microfluidic device 1825, a water-in-oil emulsion 1830 may be formed, where the emulsion contains aqueous droplets that contain sample nucleic acid, 1805, reducing agent, 1820, and barcoded beads, 1815. The reducing agent may dissolve or degrade the barcoded beads, thereby releasing the oligonucleotides with the barcodes and random N-mers from the beads within the droplets, 1835. The random N-mers may then prime different regions of the sample nucleic acid, resulting in amplified copies of the sample nucleic acid after amplification, where each copy is tagged with a barcode sequence, 1840. In some cases, amplification 1840 may be achieved by a method analogous to that described elsewhere herein and schematically depicted in FIG. 5. In some cases, each droplet contains a set of oligonucleotides that contain identical barcode sequences and different random N-mer sequences. In some cases, each droplet contains a set of oligonucleotides that contain identical barcode sequences and one or more primers directed against one or more target sequences. Subsequently, the emulsion is broken, 1845 and the barcoded sample nucleic acid fragments can be enriched for particular targets of interest. For example, barcoded sample fragments can be targeted by nucleic acid capture (e.g., hybridization to capture probes) to enrich for sequences of interest (e.g., the whole exome). In other cases, barcoded sample nucleic acid fragments can be enriched by nucleic acid amplification using primers directed to sequences of interest. Subsequent (or prior to enrichment), additional sequences (e.g., sequences that aid in particular sequencing methods, additional barcodes, etc.) may be added, via, for example, amplification methods, 1850 (e.g., PCR). Sequencing may then be performed, 1855, and an algorithm applied to interpret the sequencing data, 1860. In some cases, interpretation of the sequencing data 1860 may include providing a sequence of the sample nucleic acid. In some cases, long range sequence context for the nucleic acid sample is obtained. The sample nucleic acid sequence can be characterized 1860 (e.g., determination of one or more haplotypes as described elsewhere herein, determination of one or more structural variations (e.g., a copy number variation, an insertion, a deletion, a translocation, an inversion, a rearrangement, a repeat expansion, a duplication, retrotransposon, a gene fusion, etc.) using the characterization of the reference nucleic acid sequence 1760. Based on the comparison of the sample nucleic acid sequence and its characterization with the sequence and characterization of the reference nucleic acid, a differential diagnosis 1870 regarding the presence (or absence) of the disorder or disease state can be made.

As can be appreciated, analysis of reference nucleic acids and sample nucleic acids may completed as separate partitioning analyses or may be completed as part of a single partitioning analysis. For example, sample and reference nucleic acids may be added to the same device and barcoded sample and reference fragments generated in droplets according to FIGS. 17 and 18, where an emulsion comprises the droplets for both types of nucleic acid. The emulsion can then be broken and the contents of the droplets pooled, enriched, and further processed (e.g., bulk addition of additional sequences via PCR) and sequenced as described elsewhere herein. Individual sequencing reads from the barcoded fragments can be attributed to their respective sample sequence via barcode sequences. Sequences obtained from the sample nucleic acid can be characterized based upon the characterization of the reference nucleic acid sequence.

Utilizing methods and systems herein can improve accuracy in determining long range sequence context of nucleic acids, including the long-range sequence context of reference and sample nucleic acid sequences as described herein. The methods and systems provided herein may determine long-range sequence context of reference and/or sample nucleic acids with accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, the methods and systems provided herein may determine long-range sequence context of reference and/or sample nucleic acids with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%.

Moreover, methods and systems herein can also improve accuracy in characterizing a reference nucleic acid sequence and/or sample nucleic acid sequence in one or more aspects (e.g., determination of a sequence, determination of one or more genetic variations, determination of haplotypes, etc.). Accordingly, the methods and systems provided herein may characterize a reference nucleic acid sequence and/or sample nucleic acid sequence in one or more aspects with an accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, the methods and systems provided herein may characterize a reference nucleic acid sequence and/or sample nucleic acid sequence in one or more aspects with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%.

Moreover, as is discussed above, improved accuracy in determining long-range sequence context of reference nucleic acids and characterization of the same can result in improved accuracy in sequencing and characterizing sample nucleic acids and subsequent use in differential diagnosis of a disorder or disease. Accordingly, a sample nucleic acid sequence (including long-range sequence context) can be provided from analysis of a reference nucleic acid sequence with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. In some cases, a sample nucleic acid sequence can be used for differential diagnosis of a disorder or disease (or absence thereof) by comparison with a sequence and/or characterization of a sequence of a reference nucleic acid with accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, a sample nucleic acid sequence can be used for differential diagnosis of a disorder or disease (or absence thereof) by comparison with a sequence and/or characterization of a sequence of a reference nucleic acid with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%.

Characterizing Fetal Nucleic Acid from Parental Nucleic Acid

As noted elsewhere herein, the methods and systems described herein may also be used to characterize circulating nucleic acids within the blood or plasma of a subject. Such analyses include the analysis of circulating tumor DNA, for use in identification of potential disease states in a patient, or circulating fetal DNA within the blood or plasma of a pregnant female, in order to characterize the fetal DNA in a non-invasive way, e.g., without resorting to direct sampling through amniocentesis or other invasive procedures.

In some cases, the methods may be used to characterize fetal nucleic acid sequences, e.g. circulating fetal DNA, based, at least in part, on analysis of parental nucleic acid sequences. For example, long range sequence context can be determined for both paternal and maternal nucleic acids (e.g., having lengths of longer than 1 kb, longer than 5 kb, longer than 10 kb, longer than 15 kb, longer than 20 kb, longer than 30 kb, longer than 40 kb, longer than 50 kb, longer than 60 kb, longer than 70 kb, longer than 80 kb, longer than 90 kb or even longer than 100 kb) from shorter barcoded fragments using methods and systems described herein. Long range sequence context can be used to determine one or more haplotypes and one or more genetic variations, including single nucleotide polymorphisms (SNPs), structural variations in (e.g., a copy number variation, an insertion, a deletion, a translocation, an inversion, a rearrangement, a repeat expansion, a retrotransposon, a duplication, a gene fusion, etc.) in both the paternal and maternal nucleic acid sequences. Moreover, long range sequence context of paternal and maternal nucleic acids and any determined SNP, haplotype and/or structural variation information can be used to characterize a sequence of a fetal nucleic acid obtained from the pregnant mother (e.g., circulating fetal nucleic acid, such as, for example, cell-free fetal nucleic acid). In some cases, characterizations of a fetal nucleic acid, via comparison with maternal and paternal sequences, may be completed with the aid of a programmed computer processor. In some cases, such a programmed computer processor can be included in a computer control system, such as in an example computer control system described elsewhere herein.

For example, a sequence and/or long range sequence context of parental and/or maternal nucleic acids may be used as a reference by which to characterize fetal nucleic acid, including a fetal nucleic acid sequence. Indeed, long range sequence context obtained by methods and systems described herein can provide improved, long range sequence context information for paternal and maternal nucleic acids from which fetal nucleic acid sequences can be characterized. In some cases, characterization of a fetal nucleic acid sequence from parental nucleic acids as references may include determining a sequence for at least a portion of a fetal nucleic acid, and/or calling one or more SNPs of a fetal nucleic acid sequence, determining one or more de novo mutations of a fetal nucleic acid sequence, determining one or more haplotypes of a fetal nucleic acid sequence, and/or determining and characterizing one or more structural variations, etc. in a sequence of the fetal nucleic acid.

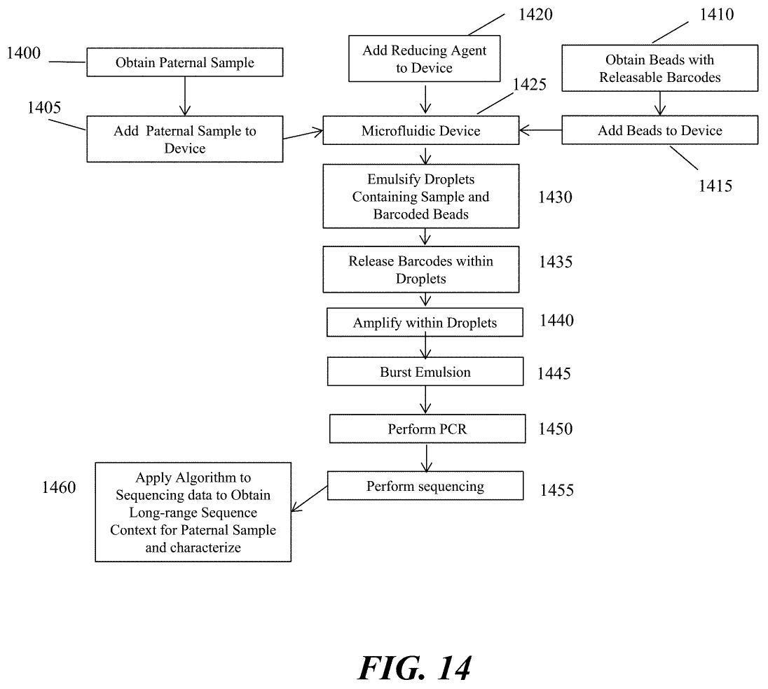

FIGS. 14-16 illustrate an example method for characterizing fetal nucleic acid from longer range sequence context obtained for paternal and maternal nucleic acid, via sequencing of shorter barcoded fragments. FIG. 14 demonstrates an example method by which longer range sequence context can be determined for a paternal nucleic acid sample (e.g., paternal genomic DNA) from shorter barcoded fragments, such as, for example, in a manner analogous to that shown in FIG. 6. With respect to FIG. 14, a sample comprising paternal nucleic acid may be obtained from the father of a fetus, 1400, and a set of barcoded beads may also be obtained, 1410. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, paternal sample comprising nucleic acid, 1405, barcoded beads, 1415, and, in some cases, other reagents, e.g., a reducing agent, 1420, are combined and subject to partitioning. In some cases, the paternal sample 1400 is fragmented prior to partitioning and at least some of the resulting fragments are partitioned as 1405 for barcoding. By way of example, such partitioning may involve introducing the components to a droplet generation system, such as a microfluidic device, 1425. With the aid of the microfluidic device 1425, a water-in-oil emulsion 1430 may be formed, where the emulsion contains aqueous droplets that contain paternal sample nucleic acid, 1405, reducing agent, 1420, and barcoded beads, 1415. The reducing agent may dissolve or degrade the barcoded beads, thereby releasing the oligonucleotides with the barcodes and random N-mers from the beads within the droplets, 1435. The random N-mers may then prime different regions of the paternal sample nucleic acid, resulting in amplified copies of the paternal sample after amplification, where each copy is tagged with a barcode sequence, 1440. In some cases, amplification 1440 may be achieved by a method analogous to that described elsewhere herein and schematically depicted in FIG. 5. In some cases, each droplet contains a set of oligonucleotides that contain identical barcode sequences and different random N-mer sequences. In other cases, each droplet contains a set of oligonucleotides that contain identical barcode sequences and one or more primer sequences directed against one or more target regions. Subsequently, the emulsion is broken, 1445 and the barcoded sample nucleic acid fragments can be enriched for particular targets of interest. For example, barcoded sample fragments can be targeted by nucleic acid capture (e.g., hybridization to capture probes) to enrich for sequences of interest (e.g., the whole exome). In other cases, barcoded sample nucleic acid fragments can be enriched by nucleic acid amplification using primers directed to sequences of interest. Subsequent (or prior to enrichment), additional sequences (e.g., sequences that aid in particular sequencing methods, additional barcodes, etc.) may be added, via, for example, amplification methods, 1450 (e.g., PCR). Sequencing may then be performed, 1455, and an algorithm applied to interpret the sequencing data 1460. In some cases, for example, interpretation of sequencing data 1460 may include providing a sequence for at least a portion of the paternal nucleic acid. In some cases, long range sequence context for the paternal nucleic acid sample can be obtained and characterized (e.g., determination of one or more haplotypes as described elsewhere herein, determination of one or more structural variations (e.g., a copy number variation, an insertion, a deletion, a translocation, an inversion, a rearrangement, a repeat expansion, a duplication, a retrotransposon, a gene fusion, etc.), calling of one or more SNPs, determination of one or more other genetic variations, etc.). In some cases, variants can be called for various paternal nucleic acids and inferred contigs generated to provide longer range sequence context, such as is described elsewhere herein with respect to FIG. 7.

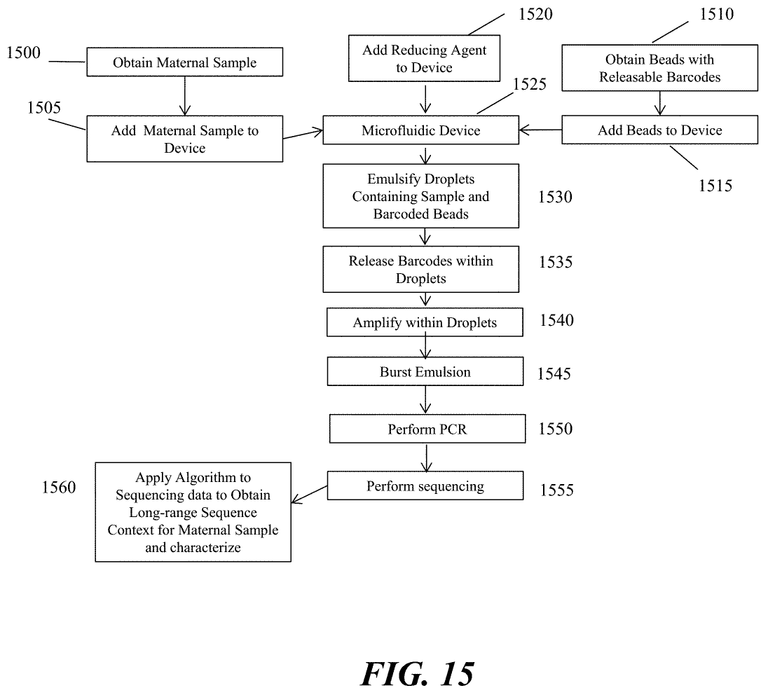

FIG. 15 demonstrates an example method by which long range sequence context can be determined for a maternal nucleic acid sample (e.g., maternal genomic DNA) from shorter barcoded fragments, such as, for example, in a manner analogous to that shown in FIG. 6. With respect to FIG. 15, a sample comprising maternal nucleic acid may be obtained from the pregnant mother of a fetus, 1500, and a set of barcoded beads may also be obtained, 1510. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, maternal sample comprising nucleic acid, 1505, barcoded beads, 1515, and, in some cases, other reagents, e.g., a reducing agent, 1520, are combined and subject to partitioning. In some cases, the maternal sample 1500 is fragmented prior to partitioning and at least some of the resulting fragments are partitioned as 1505 for barcoding. By way of example, such partitioning may involve introducing the components to a droplet generation system, such as a microfluidic device, 1525. With the aid of the microfluidic device 1525, a water-in-oil emulsion 1530 may be formed, where the emulsion contains aqueous droplets that contain maternal sample nucleic acid, 1505, reducing agent, 1520, and barcoded beads, 1515. The reducing agent may dissolve or degrade the barcoded beads, thereby releasing the oligonucleotides with the barcodes and random N-mers from the beads within the droplets, 1535. The random N-mers may then prime different regions of the maternal sample nucleic acid, resulting in amplified copies of the maternal sample after amplification, where each copy is tagged with a barcode sequence, 1540. In some cases, amplification 1540 may be achieved by a method analogous to that described elsewhere herein and schematically depicted in FIG. 5. In some cases, each droplet contains a set of oligonucleotides that contain identical barcode sequences and different random N-mer sequences. In other cases, each droplet contains a set of oligonucleotides that contain identical barcode sequences and one or more primer sequences directed against one or more target regions. Subsequently, the emulsion is broken, 1545 and the barcoded sample nucleic acid fragments can be enriched for particular targets of interest. For example, barcoded sample fragments can be targeted by nucleic acid capture (e.g., hybridization to capture probes) to enrich for sequences of interest (e.g., the whole exome). In other cases, barcoded sample nucleic acid fragments can be enriched by nucleic acid amplification using primers directed to sequences of interest. Subsequent (or prior to enrichment), additional sequences (e.g., sequences that aid in particular sequencing methods, additional barcodes, etc.) may be added, via, for example, amplification methods, 1550 (e.g., PCR). Sequencing may then be performed, 1555, and an algorithm applied to interpret the sequencing data, 1560. In some cases, for example, interpretation of sequencing data 1560 may include providing a sequence for at least a portion of the maternal nucleic acid. In some cases, long range sequence context for the maternal nucleic acid sample can be obtained and characterized (e.g., determination of one or more haplotypes as described elsewhere herein, determination of one or more structural variations (e.g., a copy number variation, an insertion, a deletion, a translocation, an inversion, a rearrangement, a repeat expansion, a duplication, a retrotransposon, a gene fusion, etc.), calling of one or more SNPs, determination of one or more other genetic variations, etc. In some cases, variants can be called for various maternal nucleic acids obtained from a sample and inferred contigs generated to provide longer range sequence context, such as is described elsewhere herein with respect to FIG. 7.

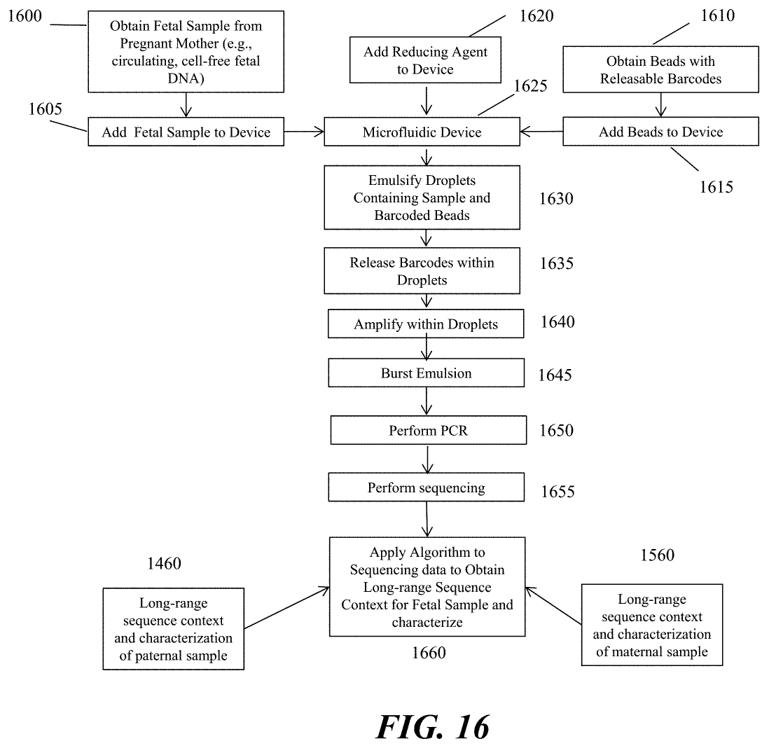

FIG. 16 demonstrates an example of characterizing a fetal sample sequence from the paternal 1460 and maternal 1560 characterizations obtained as shown in FIG. 14 and FIG. 15, respectively. As shown in FIG. 16, a fetal nucleic acid sample can be obtained from the pregnant mother 1600. Long range sequence context can be obtained for the fetal nucleic acid from sequencing of shorter barcoded fragments as is described elsewhere herein, such as, for example, via the method schematically depicted in FIG. 6. In some cases, the fetal nucleic acid sample may be circulating fetal DNA and/or cell-free DNA that may be, for example, obtained from the pregnant mother's blood, plasma, other bodily fluid, or tissue. A set of barcoded beads may also be obtained, 1610. The beads are can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some aspects, the barcoded beads can be degraded or dissolved by an agent, such as a reducing agent to release the barcode sequences. In this example, fetal sample comprising nucleic acid, 1605, barcoded beads, 1615, and, in some cases, other reagents, e.g., a reducing agent, 1620, are combined and subject to partitioning as 1605. In some cases, the fetal sample 1600 is fragmented prior to partitioning and at least some of the resulting fragments are partitioned as 1605 for barcoding. By way of example, such partitioning may involve introducing the components to a droplet generation system, such as a microfluidic device, 1625. With the aid of the microfluidic device 1625, a water-in-oil emulsion 1630 may be formed, where the emulsion contains aqueous droplets that contain maternal sample nucleic acid, 1605, reducing agent, 1620, and barcoded beads, 1615. The reducing agent may dissolve or degrade the barcoded beads, thereby releasing the oligonucleotides with the barcodes and random N-mers from the beads within the droplets, 1635. The random N-mers may then prime different regions of the fetal sample nucleic acid, resulting in amplified copies of the fetal sample after amplification, where each copy is tagged with a barcode sequence, 1640. In some cases, amplification 1640 may be achieved by a method analogous to that described elsewhere herein and schematically depicted in FIG. 5. In some cases, each droplet contains a set of oligonucleotides that contain identical barcode sequences and different random N-mer sequences. In other cases, each droplet contains a set of oligonucleotides that contain identical barcode sequences and one or more primer sequences directed against one or more target regions. Subsequently, the emulsion is broken, 1645 and the barcoded sample nucleic acid fragments can be enriched for particular targets of interest. For example, barcoded sample fragments can be targeted by nucleic acid capture (e.g., hybridization to capture probes) to enrich for sequences of interest (e.g., the whole exome). In other cases, barcoded sample nucleic acid fragments can be enriched by nucleic acid amplification using primers directed to sequences of interest. Subsequent (or prior to enrichment), additional sequences (e.g., sequences that aid in particular sequencing methods, additional barcodes, etc.) may be added, via, for example, amplification methods, 1650 (e.g., PCR). Sequencing may then be performed, 1655, and an algorithm applied to interpret the sequencing data, 1660. In general, longer range sequence context for the fetal nucleic acid sample can be obtained from the shorter barcoded fragments that are sequenced. In some cases, for example, interpretation of sequencing data 1660 may include providing a sequence for at least a portion of the fetal nucleic acid. The fetal nucleic acid sequence can be characterized 1660 (e.g., determination of one or more haplotypes as described elsewhere herein, determination of one or more structural variations (e.g., a copy number variation, an insertion, a deletion, a translocation, an inversion, a rearrangement, a repeat expansion, a duplication, retrotransposon, a gene fusion, etc.), determination of one or more de novo mutations, calling of one or more SNPs, etc.) using the long-range sequence contexts and/or characterizations of the paternal 1460 and maternal 1560 samples. In some cases, phase blocks of the fetal nucleic acid can be determined by comparison of the fetal nucleic acid sequence to the maternal and paternal phase blocks.

As can be appreciated, analysis of paternal nucleic acid, maternal nucleic acid and/or fetal nucleic acid may completed as part of separate partitioning analyses or may be completed as part of one or more combined partitioning analyses. For example, paternal, maternal and fetal nucleic acids may be added to the same device and barcoded maternal, paternal and fetal fragments generated in droplets according to FIGS. 14-16, where an emulsion comprises the droplets for the three types of nucleic acid. The emulsion can then be broken and the contents of the droplets pooled, further processed (e.g., bulk addition of additional sequences via PCR) and sequenced as described elsewhere herein. Individual sequencing reads from the barcoded fragments can be attributed to their respective sample sequence via barcode sequences.

In some cases, the sequence of a fetal nucleic acid, including the sequence of the fetal genome, and/or genetic variations in the fetal nucleic acid sequence may be determined from long range paternal and maternal sequence contexts and characterizations obtained using methods and systems described herein. For example, genome sequencing of paternal and maternal genomes, along with sequencing of circulating fetal nucleic acids, may be used to determine a corresponding fetal genome sequence. An example of determining a sequence of genomic fetal nucleic acid from sequence analysis of parental genomes and cell-free fetal nucleic acid can be found in Kitzman et al. (2012 Jun. 6) Sci Transl. Med. 4(137): 137ra76, which is herein entirely incorporated by reference. Determination of a fetal genome may be useful in the prenatal determination and diagnosis of genetic disorders in the fetus, including, for example, fetal aneuploidy. As discussed elsewhere herein, methods and systems provided herein can be useful in resolving haplotypes in nucleic acid sequences. Haplotype-resolved paternal and maternal sequences can be determined for paternal and maternal sample nucleic acid sequences, respectively which can aid in more accurately determining the sequence of a fetal genome and/or characterizing the same.

Utilizing methods and systems herein can improve accuracy in determining long range sequence context of nucleic acids, including the long-range sequence context of parental nucleic acid sequences (e.g., maternal nucleic acid sequences, paternal nucleic acid sequences). The methods and systems provided herein may determine long-range sequence context of parental nucleic acids with accuracy of at least 70%, 80%, 85%, 90%, 91%, 92%, 93%, 94%, 95%, 99%, 99.1%, 99.2%, 99.3% 99.4%, 99.5%, 99.6%, 99.7%, 99.8%, 99.9%, 99.95%, 99.99%, 99.995%, or 99.999%. In some cases, the methods and systems provided herein may determine long-range sequence context of parental nucleic acids with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. Moreover, methods and systems herein can also improve accuracy in characterizing a paternal nucleic acid sequence in one or more aspects (e.g., determination of a sequence, determination of one or more genetic variations, determination of one or more structural variants, determination of haplotypes, etc.). Accordingly, the methods and systems provided herein may characterize a paternal nucleic acid sequence in one or more aspects with an accuracy of at least 70%, 80%, 85%, 90%, 91%, 92%, 93%, 94%, 95%, 99%, 99.1%, 99.2%, 99.3° A 99.4%, 99.5%, 99.6%, 99.7%, 99.8%, 99.9%, 99.95%, 99.99%, 99.995%, or 99.999%. In some cases, the methods and systems provided herein may characterize a parental nucleic acid sequence in one or more aspects with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%.

Moreover, as is discussed above, improved accuracy in determining long-range sequence context of parental nucleic acids and characterization of the same can result in improved accuracy in sequencing and characterizing fetal nucleic acids. Accordingly, in some cases, a fetal nucleic acid sequence (including long-range sequence context) can be provided from analysis of parental nucleic sequences with accuracy of at least 70%, 80%, 85%, 90%, 91%, 92%, 93%, 94%, 95%, 99%, 99.1%, 99.2%, 99.3° A 99.4%, 99.5%, 99.6%, 99.7%, 99.8%, 99.9%, 99.95%, 99.99%, 99.995%, or 99.999%. In some cases, a fetal nucleic acid sequence (including long-range sequence context) can be provided from analysis of parental nucleic sequences with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. In some cases, a fetal nucleic acid sequence can be characterized in one or more aspects via analysis of parental nucleic acid sequences as described herein (e.g., determination of a sequence, determination of one or more genetic variations, determination of one or more structural variations, determination of haplotypes, etc.) with accuracy of at least 70%, 80%, 85%, 90%, 91%, 92%, 93%, 94%, 95%, 99%, 99.1%, 99.2%, 99.3° A 99.4%, 99.5%, 99.6%, 99.7%, 99.8%, 99.9%, 99.95%, 99.99%, 99.995%, or 99.999%. In some cases, a fetal nucleic acid sequence can be characterized in one or more aspects via analysis of parental nucleic acid sequences as described herein (e.g., determination of a sequence, determination of one or more genetic variations, determination of haplotypes, determination of one or more structural variations, etc.) with an error rate of less than 10%, 9%, 8%, 7%, 6%, 5%, 4%, 3%, 2%, 1%, 0.1%, 0.05%, 0.01%, 0.005%, 0.001%, 0.0005%, 0.0001%, 0.00005%, 0.00001%, or 0.000005%. Samples

Detection of a disease or disorder may begin with obtaining a sample from a patient. The term “sample,” as used herein, generally refers to a biological sample. Examples of biological samples include nucleic acid molecules, amino acids, polypeptides, proteins, carbohydrates, fats, or viruses. In an example, a biological sample is a nucleic acid sample including one or more nucleic acid molecules. Example samples may include polynucleotides, nucleic acids, oligonucleotides, cell-free nucleic acid (e.g., cell-free DNA (cfDNA)), circulating cell-free nucleic acid, circulating tumor nucleic acid (e.g., circulating tumor DNA (ctDNA)), circulating tumor cell (CTC) nucleic acids, nucleic acid fragments, nucleotides, DNA, RNA, peptide polynucleotides, complementary DNA (cDNA), double stranded DNA (dsDNA), single stranded DNA (ssDNA), plasmid DNA, cosmid DNA, chromosomal DNA, genomic DNA (gDNA), viral DNA, bacterial DNA, mtDNA (mitochondrial DNA), ribosomal RNA, cell-free DNA, cell free fetal DNA (cffDNA), mRNA, rRNA, tRNA, nRNA, siRNA, snRNA, snoRNA, scaRNA, microRNA, dsRNA, viral RNA, and the like. In summary, the samples that are used may vary depending on the particular processing needs.

Any substance that comprises nucleic acid may be the source of a sample. The substance may be a fluid, e.g., a biological fluid. A fluidic substance may include, but not limited to, blood, cord blood, saliva, urine, sweat, serum, semen, vaginal fluid, gastric and digestive fluid, spinal fluid, placental fluid, cavity fluid, ocular fluid, serum, breast milk, lymphatic fluid, plasma, or combinations thereof. The substance may be solid, for example, a biological tissue. The substance may comprise normal healthy tissues, diseased tissues, or a mix of healthy and diseased tissues. In some cases, the substance may comprise tumors. Tumors may be benign (non-cancer) or malignant (cancer). Non-limiting examples of tumors may include : fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's , leiomyosarcoma, rhabdomyosarcoma, gastrointestinal system carcinomas, colon carcinoma, pancreatic cancer, breast cancer, genitourinary system carcinomas, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, endocrine system carcinomas, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, or combinations thereof. The substance may be associated with various types of organs. Non-limiting examples of organs may include brain, liver, lung, kidney, prostate, ovary, spleen, lymph node (including tonsil), thyroid, pancreas, heart, skeletal muscle, intestine, larynx, esophagus, stomach, or combinations thereof. In some cases, the substance may comprise a variety of cells, including but not limited to: eukaryotic cells, prokaryotic cells, fungi cells, heart cells, lung cells, kidney cells, liver cells, pancreas cells, reproductive cells, stem cells, induced pluripotent stem cells, gastrointestinal cells, blood cells, cancer cells, bacterial cells, bacterial cells isolated from a human microbiome sample, etc. In some cases, the substance may comprise contents of a cell, such as, for example, the contents of a single cell or the contents of multiple cells. Methods and systems for analyzing individual cells are provided in, e.g., U.S. Patent Publication No. 2015/0376609, filed Jun. 26, 2015, the full disclosure of which is hereby incorporated by reference in its entirety.

Samples may be obtained from various subjects. A subject may be a living subject or a dead subject. Examples of subjects may include, but not limited to, humans, mammals, non-human mammals, rodents, amphibians, reptiles, canines, felines, bovines, equines, goats, ovines, hens, avines, mice, rabbits, insects, slugs, microbes, bacteria, parasites, or fish. In some cases, the subject may be a patient who is having, suspected of having, or at a risk of developing a disease or disorder. In some cases, the subject may be a pregnant woman. In some case, the subject may be a normal healthy pregnant woman. In some cases, the subject may be a pregnant woman who is at a risking of carrying a baby with certain birth defect.

When the subject is a pregnant woman, the sample may be referred to as a maternal biological sample. The maternal biological sample can be a blood sample. The maternal biological sample can be a maternal cell-free biological sample. The maternal cell-free biological sample can be a plasma sample. The maternal biological sample can comprise maternal nucleic acid sequences. In some cases, the maternal biological sample further comprises fetal nucleic acid sequences. The subject can be a male who is the father of a fetus. When the subject is a male who is the father of a fetus, the sample may be referred to as a paternal biological sample. The paternal biological sample can comprise paternal nucleic acid sequences.

A sample may be obtained from a subject by various approaches. For example, a sample may be obtained from a subject through accessing the circulatory system (e.g., intravenously or intra-arterially via a syringe or other apparatus), collecting a secreted biological sample (e.g., saliva, sputum urine, feces, etc.), surgically (e.g., biopsy) acquiring a biological sample (e.g., intra-operative samples, post-surgical samples, etc.), swabbing (e.g., buccal swab, oropharyngeal swab), or pipetting.

CNVs can be associated with efficacy of a therapy. For example, increased HER2 gene copy number can enhance the response to gefitinib therapy in advanced non-small cell lung cancer. See Cappuzzo F. et al. (2005) J. Clin. Oncol. 23: 5007-5018. High EGFR gene copy number can predict for increased sensitivity to lapatinib and capecitabine. See Fabi et al. (2010) J. Clin. Oncol. 28:15s (2010 ASCO Annual Meeting). High EGFR gene copy number is associated with increased sensitivity to cetuximab and panitumumab.

Copy number variations can be associated with resistance of cancer patients to certain therapeutics. For example, amplification of thymidylate synthase can result in resistance to 5-fluorouracil treatment in metastatic colorectal cancer patients. See Wang et al. (2002) PNAS USA vol. 99, pp. 16156-61.

### Systems and Methods for Sample Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. The partition can be a droplet in an emulsion. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead. A cell bead can be a biological particle and/or one or more of its macromolecular constituents encased inside of a gel or polymer matrix, such as via polymerization of a droplet containing the biological particle and precursors capable of being polymerized or gelled. Unique identifiers, such as barcodes, may be injected into the droplets previous to, subsequent to, or concurrently with droplet generation, such as via a microcapsule (e.g., bead), as described elsewhere herein. Microfluidic channel networks (e.g., on a chip) can be utilized to generate partitions as described herein. Alternative mechanisms may also be employed in the partitioning of individual biological particles, including porous membranes through which aqueous mixtures of cells are extruded into non-aqueous fluids.

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In the case of droplets in an emulsion, allocating individual particles to discrete partitions may in one non-limiting example be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

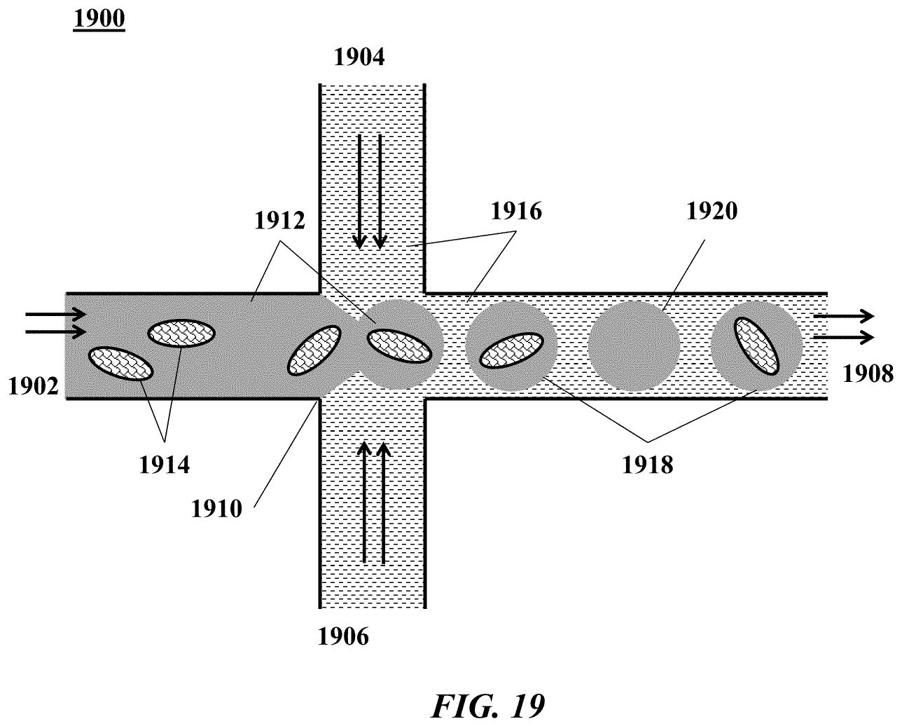

FIG. 19 shows an example of a microfluidic channel structure 1900 for partitioning individual biological particles. The channel structure 1900 can include channel segments 1902, 1904, 1906 and 1908 communicating at a channel junction 1910. In operation, a first aqueous fluid 1912 that includes suspended biological particles (or cells) 1914 may be transported along channel segment 1902 into junction 1910, while a second fluid 1916 that is immiscible with the aqueous fluid 1912 is delivered to the junction 1910 from each of channel segments 1904 and 1906 to create discrete droplets 1918, 1920 of the first aqueous fluid 1912 flowing into channel segment 1908, and flowing away from junction 1910. The channel segment 1908 may be fluidically coupled to an outlet reservoir where the discrete droplets can be stored and/or harvested. A discrete droplet generated may include an individual biological particle 1914 (such as droplets 1918). A discrete droplet generated may include more than one individual biological particle 1914 (not shown in FIG. 19). A discrete droplet may contain no biological particle 1914 (such as droplet 1920). Each discrete partition may maintain separation of its own contents (e.g., individual biological particle 1914) from the contents of other partitions.

The second fluid 1916 can comprise an oil, such as a fluorinated oil, that includes a fluorosurfactant for stabilizing the resulting droplets, for example, inhibiting subsequent coalescence of the resulting droplets 1918, 1920. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 1900 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 1918, containing one or more biological particles 1914, and (2) unoccupied droplets 1920, not containing any biological particles 1914. Occupied droplets 1918 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 1914) at the partitioning junction 1910, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles (e.g., in channel segment 1902), or other fluids directed into the partitioning junction (e.g., in channel segments 1904, 1906) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of singly-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

As will be appreciated, the above-described occupancy rates are also applicable to partitions that include both biological particles and additional reagents, including, but not limited to, microcapsules or beads (e.g., gel beads) carrying barcoded nucleic acid molecules (e.g., oligonucleotides). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid molecules and a biological particle.

In another aspect, in addition to or as an alternative to droplet based partitioning, biological particles may be encapsulated within a microcapsule that comprises an outer shell, layer or porous matrix in which is entrained one or more individual biological particles or small groups of biological particles. The microcapsule may include other reagents. Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Preparation of microcapsules comprising biological particles may be performed by a variety of methods. For example, air knife droplet or aerosol generators may be used to dispense droplets of precursor fluids into gelling solutions in order to form microcapsules that include individual biological particles or small groups of biological particles. Likewise, membrane based encapsulation systems may be used to generate microcapsules comprising encapsulated biological particles as described herein. Microfluidic systems of the present disclosure, such as that shown in FIG. 19, may be readily used in encapsulating cells as described herein. In particular, and with reference to FIG. 19, the aqueous fluid 1912 comprising (i) the biological particles 1914 and (ii) the polymer precursor material (not shown) is flowed into channel junction 1910, where it is partitioned into droplets 1918, 1920 through the flow of non-aqueous fluid 1916. In the case of encapsulation methods, non-aqueous fluid 1916 may also include an initiator (not shown) to cause polymerization and/or crosslinking of the polymer precursor to form the microcapsule that includes the entrained biological particles. Examples of polymer precursor/initiator pairs include those described in U.S. Patent Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams 1916 in channel segments 1904 and 1906, which can initiate the copolymerization of the acrylamide and BAC into a cross-linked polymer network, or hydrogel.

Upon contact of the second fluid stream 1916 with the first fluid stream 1912 at junction 1910, during formation of droplets, the TEMED may diffuse from the second fluid 1916 into the aqueous fluid 1912 comprising the linear polyacrylamide, which will activate the crosslinking of the polyacrylamide within the droplets 1918, 1920, resulting in the formation of gel (e.g., hydrogel) microcapsules, as solid or semi-solid beads or particles entraining the cells 1914. Although described in terms of polyacrylamide encapsulation, other ‘activatable’ encapsulation compositions may also be employed in the context of the methods and compositions described herein. For example, formation of alginate droplets followed by exposure to divalent metal ions (e.g., Ca2+ ions), can be used as an encapsulation process using the described processes. Likewise, agarose droplets may also be transformed into capsules through temperature based gelling (e.g., upon cooling, etc.).

In some cases, encapsulated biological particles can be selectively releasable from the microcapsule, such as through passage of time or upon application of a particular stimulus, that degrades the microcapsule sufficiently to allow the biological particles (e.g., cell), or its other contents to be released from the microcapsule, such as into a partition (e.g., droplet). For example, in the case of the polyacrylamide polymer described above, degradation of the microcapsule may be accomplished through the introduction of an appropriate reducing agent, such as DTT or the like, to cleave disulfide bonds that cross-link the polymer matrix. See, for example, U.S. Patent Application Publication No. 2014/0378345, which is entirely incorporated herein by reference for all purposes.

The biological particle can be subjected to other conditions sufficient to polymerize or gel the precursors. The conditions sufficient to polymerize or gel the precursors may comprise exposure to heating, cooling, electromagnetic radiation, and/or light. The conditions sufficient to polymerize or gel the precursors may comprise any conditions sufficient to polymerize or gel the precursors. Following polymerization or gelling, a polymer or gel may be formed around the biological particle. The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel. The polymer or gel may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The polymer may comprise poly(acrylamide-co-acrylic acid) crosslinked with disulfide linkages. The preparation of the polymer may comprise a two-step reaction. In the first activation step, poly(acrylamide-co-acrylic acid) may be exposed to an acylating agent to convert carboxylic acids to esters. For instance, the poly(acrylamide-co-acrylic acid) may be exposed to 4-(4,6-dimethoxy-1,3,5-triazin-2-yl)-4-methylmorpholinium chloride (DMTMM). The polyacrylamide-co-acrylic acid may be exposed to other salts of 4-(4,6-dimethoxy-1,3,5-triazin-2-yl)-4-methylmorpholinium. In the second cross-linking step, the ester formed in the first step may be exposed to a disulfide crosslinking agent. For instance, the ester may be exposed to cystamine (2,2′-dithiobis(ethylamine)). Following the two steps, the biological particle may be surrounded by polyacrylamide strands linked together by disulfide bridges. In this manner, the biological particle may be encased inside of or comprise a gel or matrix (e.g., polymer matrix) to form a “cell bead.” A cell bead can contain biological particles (e.g., a cell) or macromolecular constituents (e.g., RNA, DNA, proteins, etc.) of biological particles. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles.

Encapsulated biological particles can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli. In such cases, encapsulation may allow for longer incubation than partitioning in emulsion droplets, although in some cases, droplet partitioned biological particles may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles may constitute the partitioning of the biological particles into which other reagents are co-partitioned. Alternatively or in addition, encapsulated biological particles may be readily deposited into other partitions (e.g., droplets) as described above. Beads

A partition may comprise one or more unique identifiers, such as barcodes. Barcodes may be previously, subsequently or concurrently delivered to the partitions that hold the compartmentalized or partitioned biological particle. For example, barcodes may be injected into droplets previous to, subsequent to, or concurrently with droplet generation. The delivery of the barcodes to a particular partition allows for the later attribution of the characteristics of the individual biological particle to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead. Beads are described in further detail below.

In some cases, barcoded nucleic acid molecules can be initially associated with the microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

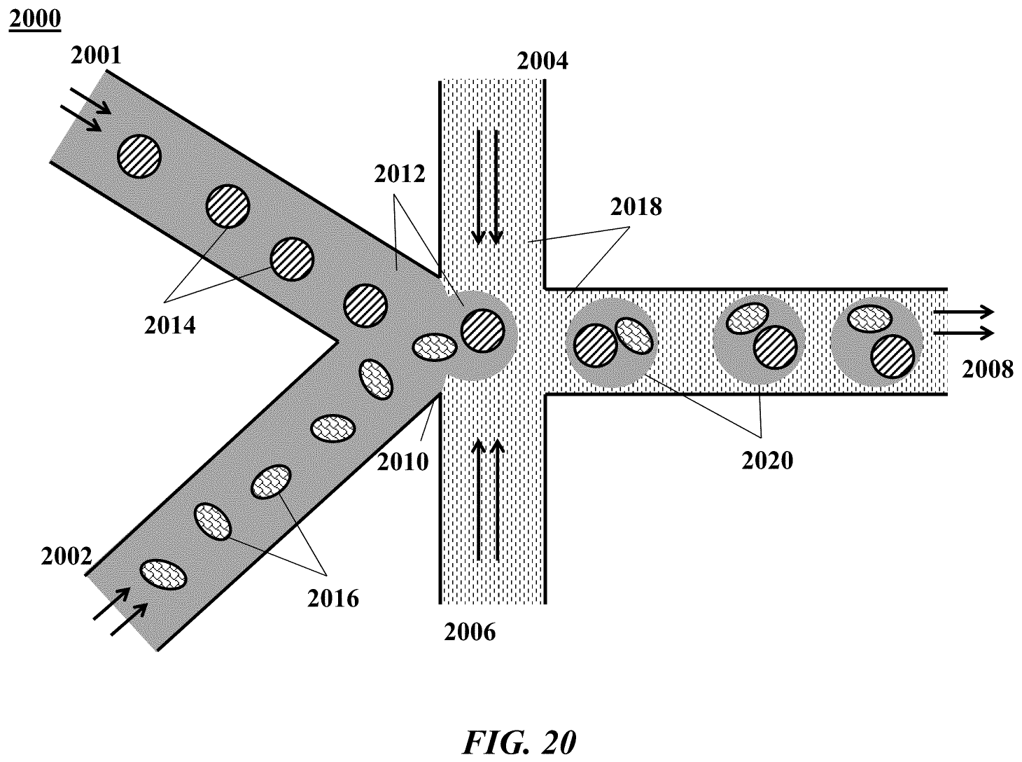

FIG. 20 shows an example of a microfluidic channel structure 2000 for delivering barcode carrying beads to droplets. The channel structure 2000 can include channel segments 2001, 2002, 2004, 2006 and 2008 communicating at a channel junction 2010. In operation, the channel segment 2001 may transport an aqueous fluid 2012 that includes a plurality of beads 2014 (e.g., with nucleic acid molecules, oligonucleotides, molecular tags) along the channel segment 2001 into junction 2010. The plurality of beads 2014 may be sourced from a suspension of beads. For example, the channel segment 2001 may be connected to a reservoir comprising an aqueous suspension of beads 2014. The channel segment 2002 may transport the aqueous fluid 2012 that includes a plurality of biological particles 2016 along the channel segment 2002 into junction 2010. The plurality of biological particles 2016 may be sourced from a suspension of biological particles. For example, the channel segment 2002 may be connected to a reservoir comprising an aqueous suspension of biological particles 2016. In some instances, the aqueous fluid 2012 in either the first channel segment 2001 or the second channel segment 2002, or in both segments, can include one or more reagents, as further described below. A second fluid 2018 that is immiscible with the aqueous fluid 2012 (e.g., oil) can be delivered to the junction 2010 from each of channel segments 2004 and 2006. Upon meeting of the aqueous fluid 2012 from each of channel segments 2001 and 2002 and the second fluid 2018 from each of channel segments 2004 and 2006 at the channel junction 2010, the aqueous fluid 2012 can be partitioned as discrete droplets 2020 in the second fluid 2018 and flow away from the junction 2010 along channel segment 2008. The channel segment 2008 may deliver the discrete droplets to an outlet reservoir fluidly coupled to the channel segment 2008, where they may be harvested.

As an alternative, the channel segments 2001 and 2002 may meet at another junction upstream of the junction 2010. At such junction, beads and biological particles may form a mixture that is directed along another channel to the junction 2010 to yield droplets 2020. The mixture may provide the beads and biological particles in an alternating fashion, such that, for example, a droplet comprises a single bead and a single biological particle.

Beads, biological particles and droplets may flow along channels at substantially regular flow profiles (e.g., at regular flow rates). Such regular flow profiles may permit a droplet to include a single bead and a single biological particle. Such regular flow profiles may permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that may be used to provide such regular flow profiles are provided in, for example, U.S. Patent Publication No. 2015/0292988, which is entirely incorporated herein by reference.

The second fluid 2018 can comprise an oil, such as a fluorinated oil, that includes a fluorosurfactant for stabilizing the resulting droplets, for example, inhibiting subsequent coalescence of the resulting droplets 2020.

A discrete droplet that is generated may include an individual biological particle 2016. A discrete droplet that is generated may include a barcode or other reagent carrying bead 2014. A discrete droplet generated may include both an individual biological particle and a barcode carrying bead, such as droplets 2020. In some instances, a discrete droplet may include more than one individual biological particle or no biological particle. In some instances, a discrete droplet may include more than one bead or no bead. A discrete droplet may be unoccupied (e.g., no beads, no biological particles).

Beneficially, a discrete droplet partitioning a biological particle and a barcode carrying bead may effectively allow the attribution of the barcode to macromolecular constituents of the biological particle within the partition. The contents of a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 2000 may have other geometries. For example, a microfluidic channel structure can have more than one channel junctions. For example, a microfluidic channel structure can have 2, 3, 4, or 5 channel segments each carrying beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

A bead may be porous, non-porous, solid, semi-solid, semi-fluidic, fluidic, and/or a combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds, thioether bonds, or carbon-heteroatom bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R1 primer sequence for Illumina sequencing. In some cases, the primer can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

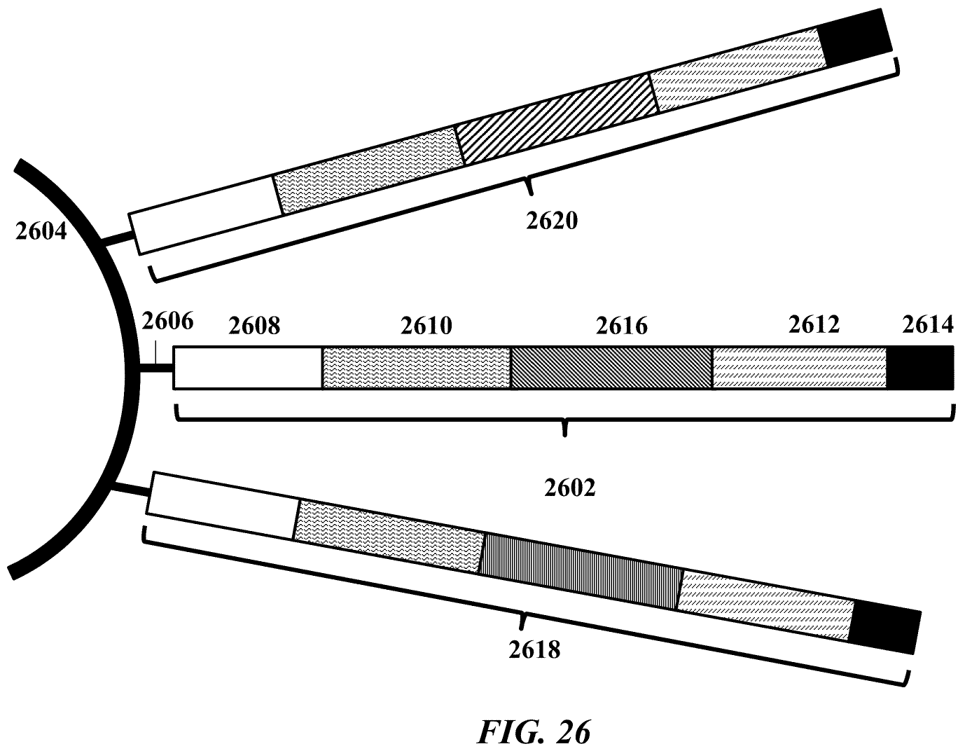

FIG. 26 illustrates an example of a barcode carrying bead. A nucleic acid molecule 802, such as an oligonucleotide, can be coupled to a bead 2604 by a releasable linkage 2606, such as, for example, a disulfide linker. The same bead 2604 may be coupled (e.g., via releasable linkage) to one or more other nucleic acid molecules 2618, 2620. The nucleic acid molecule 2602 may be or comprise a barcode. As noted elsewhere herein, the structure of the barcode may comprise a number of sequence elements. The nucleic acid molecule 2602 may comprise a functional sequence 2608 that may be used in subsequent processing. For example, the functional sequence 2608 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 2602 may comprise a barcode sequence 2610 for use in barcoding the sample (e.g., DNA, RNA, protein, etc.). In some cases, the barcode sequence 2610 can be bead-specific such that the barcode sequence 2610 is common to all nucleic acid molecules (e.g., including nucleic acid molecule 2602) coupled to the same bead 2604. Alternatively or in addition, the barcode sequence 2610 can be partition-specific such that the barcode sequence 2610 is common to all nucleic acid molecules coupled to one or more beads that are partitioned into the same partition. The nucleic acid molecule 2602 may comprise a specific priming sequence 2612, such as an mRNA specific priming sequence (e.g., poly-dT sequence), a targeted priming sequence, and/or a random priming sequence. The nucleic acid molecule 2602 may comprise an anchoring sequence 2614 to ensure that the specific priming sequence 2612 hybridizes at the sequence end (e.g., of the mRNA). For example, the anchoring sequence 2614 can include a random short sequence of nucleotides, such as a 1-mer, 2-mer, 3-mer or longer sequence, which can ensure that a poly-dT segment is more likely to hybridize at the sequence end of the poly-A tail of the mRNA.

The nucleic acid molecule 2602 may comprise a unique molecular identifying sequence 2616 (e.g., unique molecular identifier (UMI)). In some cases, the unique molecular identifying sequence 2616 may comprise from about 5 to about 8 nucleotides. Alternatively, the unique molecular identifying sequence 2616 may compress less than about 5 or more than about 8 nucleotides. The unique molecular identifying sequence 2616 may be a unique sequence that varies across individual nucleic acid molecules (e.g., 2602, 2618, 2620, etc.) coupled to a single bead (e.g., bead 2604). In some cases, the unique molecular identifying sequence 2616 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 26 shows three nucleic acid molecules 2602, 2618, 2620 coupled to the surface of the bead 2604, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 2608, 2610, 2612, etc.) and variable or unique sequence segments (e.g., 2616) between different individual nucleic acid molecules coupled to the same bead.

In operation, a biological particle (e.g., cell, DNA, RNA, etc.) can be co-partitioned along with a barcode bearing bead 2604. The barcoded nucleic acid molecules 2602, 2618, 2620 can be released from the bead 2604 in the partition. By way of example, in the context of analyzing sample RNA, the poly-dT segment (e.g., 2612) of one of the released nucleic acid molecules (e.g., 2602) can hybridize to the poly-A tail of a mRNA molecule. Reverse transcription may result in a cDNA transcript of the mRNA, but which transcript includes each of the sequence segments 2608, 2610, 2616 of the nucleic acid molecule 2602. Because the nucleic acid molecule 2602 comprises an anchoring sequence 2614, it will more likely hybridize to and prime reverse transcription at the sequence end of the poly-A tail of the mRNA. Within any given partition, all of the cDNA transcripts of the individual mRNA molecules may include a common barcode sequence segment 2610. However, the transcripts made from the different mRNA molecules within a given partition may vary at the unique molecular identifying sequence 2612 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-dT primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photo-sensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may include, but are not limited to pH-mediated changes to the integrity of a component within the bead, degradation of a component of a bead via cleavage of cross-linked bonds, and depolymerization of a component of a bead.

In some embodiments, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead. In some cases, any combination of stimuli may trigger degradation of a bead. For example, a change in pH may enable a chemical agent (e.g., DTT) to become an effective reducing agent.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

Although FIG. 19 and FIG. 20 have been described in terms of providing substantially singly occupied partitions, above, in certain cases, it may be desirable to provide multiply occupied partitions, e.g., containing two, three, four or more cells and/or microcapsules (e.g., beads) comprising barcoded nucleic acid molecules (e.g., oligonucleotides) within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction (e.g., junction 2010). In such cases, the flow and frequency of the different beads into the channel or junction may be controlled to provide for a certain ratio of microcapsules from each source, while ensuring a given pairing or combination of such beads into a partition with a given number of biological particles (e.g., one biological particle and one bead per partition).

The partitions described herein may comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with microcapsules, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions. Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone such as through an additional channel or channels upstream of the channel junction. In accordance with other aspects, additionally or alternatively, biological particles may be partitioned along with other reagents, as will be described further below.

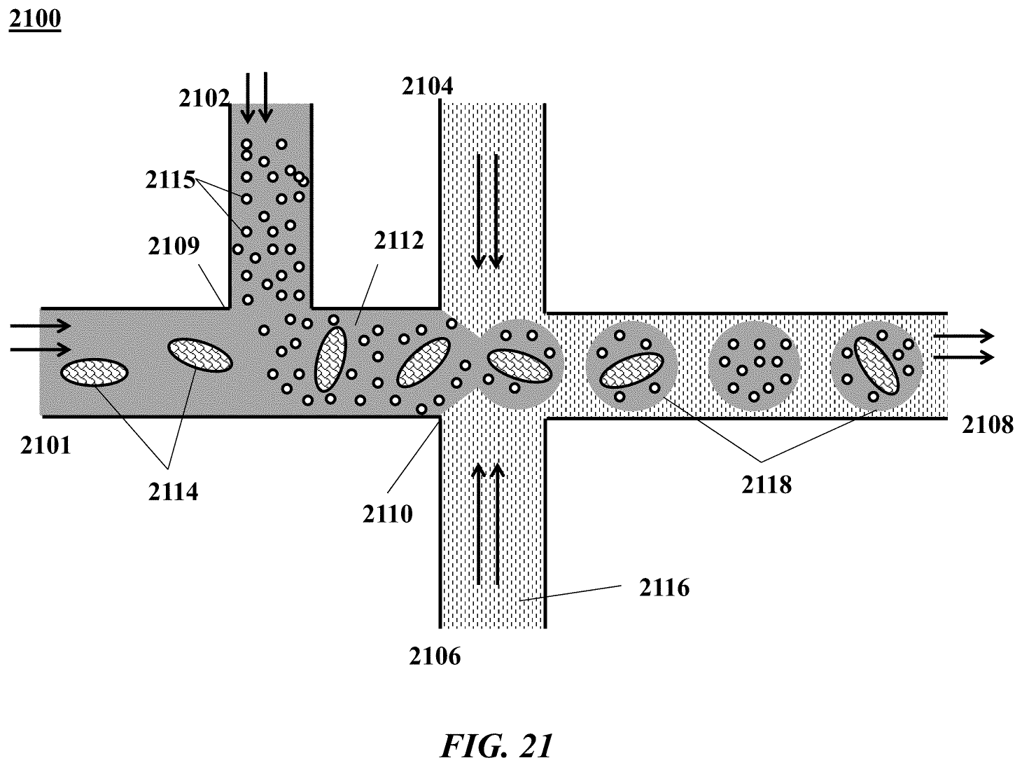

FIG. 21 shows an example of a microfluidic channel structure 2100 for co-partitioning biological particles and reagents. The channel structure 2100 can include channel segments 2101, 2102, 2104, 2106 and 2108. Channel segments 2101 and 2102 communicate at a first channel junction 2109. Channel segments 2102, 2104, 2106, and 2108 communicate at a second channel junction 2110.

In an example operation, the channel segment 2101 may transport an aqueous fluid 2112 that includes a plurality of biological particles 2114 along the channel segment 2101 into the second junction 2110. As an alternative or in addition to, channel segment 2101 may transport beads (e.g., gel beads). The beads may comprise barcode molecules.

For example, the channel segment 2101 may be connected to a reservoir comprising an aqueous suspension of biological particles 2114. Upstream of, and immediately prior to reaching, the second junction 2110, the channel segment 2101 may meet the channel segment 2102 at the first junction 2109. The channel segment 2102 may transport a plurality of reagents 2115 (e.g., lysis agents) suspended in the aqueous fluid 2112 along the channel segment 2102 into the first junction 2109. For example, the channel segment 2102 may be connected to a reservoir comprising the reagents 2115. After the first junction 2109, the aqueous fluid 2112 in the channel segment 2101 can carry both the biological particles 2114 and the reagents 2115 towards the second junction 2110. In some instances, the aqueous fluid 2112 in the channel segment 2101 can include one or more reagents, which can be the same or different reagents as the reagents 2115. A second fluid 2116 that is immiscible with the aqueous fluid 2112 (e.g., oil) can be delivered to the second junction 2110 from each of channel segments 2104 and 2106. Upon meeting of the aqueous fluid 2112 from the channel segment 2101 and the second fluid 2116 from each of channel segments 2104 and 2106 at the second channel junction 2110, the aqueous fluid 2112 can be partitioned as discrete droplets 2118 in the second fluid 2116 and flow away from the second junction 2110 along channel segment 2108. The channel segment 2108 may deliver the discrete droplets 2118 to an outlet reservoir fluidly coupled to the channel segment 2108, where they may be harvested.

The second fluid 2116 can comprise an oil, such as a fluorinated oil, that includes a fluorosurfactant for stabilizing the resulting droplets, for example, inhibiting subsequent coalescence of the resulting droplets 2118.

A discrete droplet generated may include an individual biological particle 2114 and/or one or more reagents 2115. In some instances, a discrete droplet generated may include a barcode carrying bead (not shown), such as via other microfluidics structures described elsewhere herein. In some instances, a discrete droplet may be unoccupied (e.g., no reagents, no biological particles).

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 2100 may have other geometries. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the biological particles to cause the release of the biological particles' contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles, the biological particles may be exposed to an appropriate stimulus to release the biological particles or their contents from a co-partitioned microcapsule. For example, in some cases, a chemical stimulus may be co-partitioned along with an encapsulated biological particle to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative aspects, this may be a different and non-overlapping stimulus, in order to allow an encapsulated biological particle to be released into a partition at a different time from the release of nucleic acid molecules into the same partition.

Additional reagents may also be co-partitioned with the biological particles, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 , 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 , 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle or groups of biological particles with the unique identifiers, such as described above (with reference to FIG. 20). In some aspects, the unique identifiers are provided in the form of nucleic acid molecules (e.g., oligonucleotides) that comprise nucleic acid barcode sequences that may be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides into partitions, e.g., droplets within microfluidic systems.

In an example, microcapsules, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules form the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

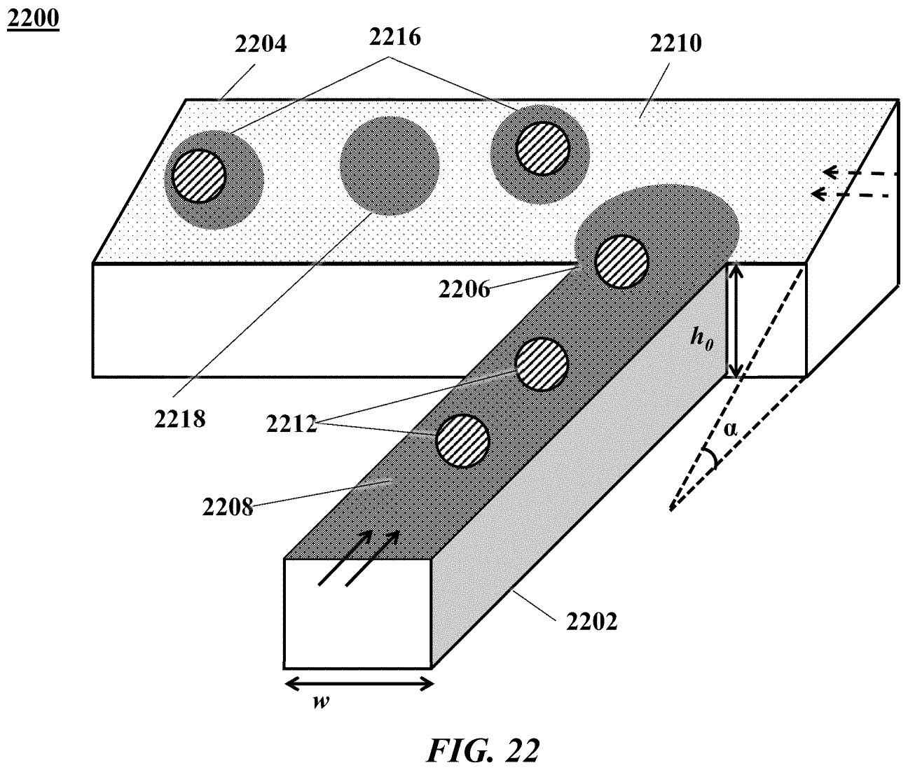

FIG. 22 shows an example of a microfluidic channel structure for the controlled partitioning of beads into discrete droplets. A channel structure 2200 can include a channel segment 2202 communicating at a channel junction 2206 (or intersection) with a reservoir 2204. The reservoir 2204 can be a chamber. Any reference to “reservoir,” as used herein, can also refer to a “chamber.” In operation, an aqueous fluid 2208 that includes suspended beads 2212 may be transported along the channel segment 2202 into the junction 2206 to meet a second fluid 2210 that is immiscible with the aqueous fluid 2208 in the reservoir 2204 to create droplets 2216, 2218 of the aqueous fluid 2208 flowing into the reservoir 2204. At the junction 2206 where the aqueous fluid 2208 and the second fluid 2210 meet, droplets can form based on factors such as the hydrodynamic forces at the junction 2206, flow rates of the two fluids 2208, 2210, fluid properties, and certain geometric parameters (e.g., w, h₀, α, etc.) of the channel structure 2200. A plurality of droplets can be collected in the reservoir 2204 by continuously injecting the aqueous fluid 2208 from the channel segment 2202 through the junction 2206.

A discrete droplet generated may include a bead (e.g., as in occupied droplets 2216). Alternatively, a discrete droplet generated may include more than one bead. Alternatively, a discrete droplet generated may not include any beads (e.g., as in unoccupied droplet 2218). In some instances, a discrete droplet generated may contain one or more biological particles, as described elsewhere herein. In some instances, a discrete droplet generated may comprise one or more reagents, as described elsewhere herein.

In some instances, the aqueous fluid 2208 can have a substantially uniform concentration or frequency of beads 2212. The beads 2212 can be introduced into the channel segment 2202 from a separate channel (not shown in FIG. 22). The frequency of beads 2212 in the channel segment 2202 may be controlled by controlling the frequency in which the beads 2212 are introduced into the channel segment 2202 and/or the relative flow rates of the fluids in the channel segment 2202 and the separate channel. In some instances, the beads can be introduced into the channel segment 2202 from a plurality of different channels, and the frequency controlled accordingly.

In some instances, the aqueous fluid 2208 in the channel segment 2202 can comprise biological particles (e.g., described with reference to FIGS. 19 and 20). In some instances, the aqueous fluid 2208 can have a substantially uniform concentration or frequency of biological particles. As with the beads, the biological particles can be introduced into the channel segment 2202 from a separate channel. The frequency or concentration of the biological particles in the aqueous fluid 2208 in the channel segment 2202 may be controlled by controlling the frequency in which the biological particles are introduced into the channel segment 2202 and/or the relative flow rates of the fluids in the channel segment 2202 and the separate channel. In some instances, the biological particles can be introduced into the channel segment 2202 from a plurality of different channels, and the frequency controlled accordingly. In some instances, a first separate channel can introduce beads and a second separate channel can introduce biological particles into the channel segment 2202. The first separate channel introducing the beads may be upstream or downstream of the second separate channel introducing the biological particles.

The second fluid 2210 can comprise an oil, such as a fluorinated oil, that includes a fluorosurfactant for stabilizing the resulting droplets, for example, inhibiting subsequent coalescence of the resulting droplets.

In some instances, the second fluid 2210 may not be subjected to and/or directed to any flow in or out of the reservoir 2204. For example, the second fluid 2210 may be substantially stationary in the reservoir 2204. In some instances, the second fluid 2210 may be subjected to flow within the reservoir 2204, but not in or out of the reservoir 2204, such as via application of pressure to the reservoir 2204 and/or as affected by the incoming flow of the aqueous fluid 2208 at the junction 2206. Alternatively, the second fluid 2210 may be subjected and/or directed to flow in or out of the reservoir 2204. For example, the reservoir 2204 can be a channel directing the second fluid 2210 from upstream to downstream, transporting the generated droplets.

The channel structure 2200 at or near the junction 2206 may have certain geometric features that at least partly determine the sizes of the droplets formed by the channel structure 2200. The channel segment 2202 can have a height, h₀ and width, w, at or near the junction 2206. By way of example, the channel segment 2202 can comprise a rectangular cross-section that leads to a reservoir 2204 having a wider cross-section (such as in width or diameter). Alternatively, the cross-section of the channel segment 2202 can be other shapes, such as a circular shape, trapezoidal shape, polygonal shape, or any other shapes. The top and bottom walls of the reservoir 2204 at or near the junction 2206 can be inclined at an expansion angle, α. The expansion angle, α, allows the tongue (portion of the aqueous fluid 2208 leaving channel segment 2202 at junction 2206 and entering the reservoir 2204 before droplet formation) to increase in depth and facilitate decrease in curvature of the intermediately formed droplet. Droplet size may decrease with increasing expansion angle. The resulting droplet radius, R_{d}, may be predicted by the following equation for the aforementioned geometric parameters of h₀, w, and α:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 μm, h=25 μm, and α=5°, the predicted droplet size is 123 μm. In another example, for a channel structure with w=28 μm, h=28 μm, and α=7°, the predicted droplet size is 124 μm.

In some instances, the expansion angle, α, may be between a range of from about 0.5° to about 4°, from about 0.1° to about 10° , or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher.

In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 2208 entering the junction 2206 can be between about 0.04 microliters (μL)/minute (min) and about 40 μL/min. In some instances, the flow rate of the aqueous fluid 2208 entering the junction 2206 can be between about 0.01 microliters (μL)/minute (min) and about 100 μL/min. Alternatively, the flow rate of the aqueous fluid 2208 entering the junction 2206 can be less than about 0.01 μL/min. Alternatively, the flow rate of the aqueous fluid 2208 entering the junction 2206 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 2208 entering the junction 2206.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 2206) between aqueous fluid 2208 channel segments (e.g., channel segment 2202) and the reservoir 2204. Alternatively or in addition, the throughput of droplet generation can be increased by increasing the flow rate of the aqueous fluid 2208 in the channel segment 2202.

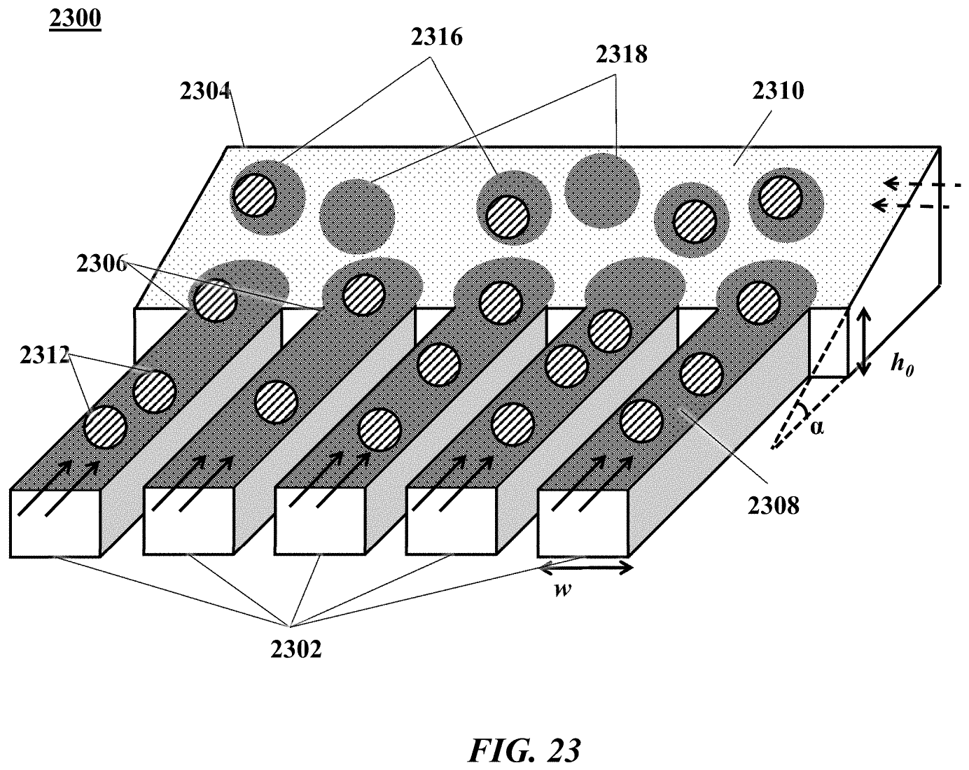

FIG. 23 shows an example of a microfluidic channel structure for increased droplet generation throughput. A microfluidic channel structure 2300 can comprise a plurality of channel segments 2302 and a reservoir 2304. Each of the plurality of channel segments 2302 may be in fluid communication with the reservoir 2304. The channel structure 2300 can comprise a plurality of channel junctions 2306 between the plurality of channel segments 2302 and the reservoir 2304. Each channel junction can be a point of droplet generation. The channel segment 2202 from the channel structure 2200 in FIG. 22 and any description to the components thereof may correspond to a given channel segment of the plurality of channel segments 2302 in channel structure 2300 and any description to the corresponding components thereof. The reservoir 2304 from the channel structure 2200 and any description to the components thereof may correspond to the reservoir 2304 from the channel structure 2300 and any description to the corresponding components thereof.

Each channel segment of the plurality of channel segments 2302 may comprise an aqueous fluid 2308 that includes suspended beads 2312. The reservoir 2304 may comprise a second fluid 2310 that is immiscible with the aqueous fluid 2308. In some instances, the second fluid 2310 may not be subjected to and/or directed to any flow in or out of the reservoir 2304. For example, the second fluid 2310 may be substantially stationary in the reservoir 2304. In some instances, the second fluid 2310 may be subjected to flow within the reservoir 2304, but not in or out of the reservoir 2304, such as via application of pressure to the reservoir 2304 and/or as affected by the incoming flow of the aqueous fluid 2308 at the junctions. Alternatively, the second fluid 2310 may be subjected and/or directed to flow in or out of the reservoir 2304. For example, the reservoir 2304 can be a channel directing the second fluid 2310 from upstream to downstream, transporting the generated droplets.

In operation, the aqueous fluid 2308 that includes suspended beads 2312 may be transported along the plurality of channel segments 2302 into the plurality of junctions 2306 to meet the second fluid 2310 in the reservoir 2304 to create droplets 2316, 2318. A droplet may form from each channel segment at each corresponding junction with the reservoir 2304. At the junction where the aqueous fluid 2308 and the second fluid 2310 meet, droplets can form based on factors such as the hydrodynamic forces at the junction, flow rates of the two fluids 2308, 2310, fluid properties, and certain geometric parameters (e.g., w, h₀, α, etc.) of the channel structure 2300, as described elsewhere herein. A plurality of droplets can be collected in the reservoir 2304 by continuously injecting the aqueous fluid 2308 from the plurality of channel segments 2302 through the plurality of junctions 2306. Throughput may significantly increase with the parallel channel configuration of channel structure 2300. For example, a channel structure having five inlet channel segments comprising the aqueous fluid 2308 may generate droplets five times as frequently than a channel structure having one inlet channel segment, provided that the fluid flow rate in the channel segments are substantially the same. The fluid flow rate in the different inlet channel segments may or may not be substantially the same. A channel structure may have as many parallel channel segments as is practical and allowed for the size of the reservoir. For example, the channel structure may have at least about 2, 3, 4, 5, 6, 7, 8, 9, 10, 20, 30, 40, 50, 60, 70, 80, 90, 100, 150, 500, 250, 300, 350, 400, 450, 500, 600, 700, 800, 900, 1000, 1500, 5000 or more parallel or substantially parallel channel segments.

The geometric parameters, w, h₀, and α, may or may not be uniform for each of the channel segments in the plurality of channel segments 2302. For example, each channel segment may have the same or different widths at or near its respective channel junction with the reservoir 2304. For example, each channel segment may have the same or different height at or near its respective channel junction with the reservoir 2304. In another example, the reservoir 2304 may have the same or different expansion angle at the different channel junctions with the plurality of channel segments 2302. When the geometric parameters are uniform, beneficially, droplet size may also be controlled to be uniform even with the increased throughput. In some instances, when it is desirable to have a different distribution of droplet sizes, the geometric parameters for the plurality of channel segments 2302 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

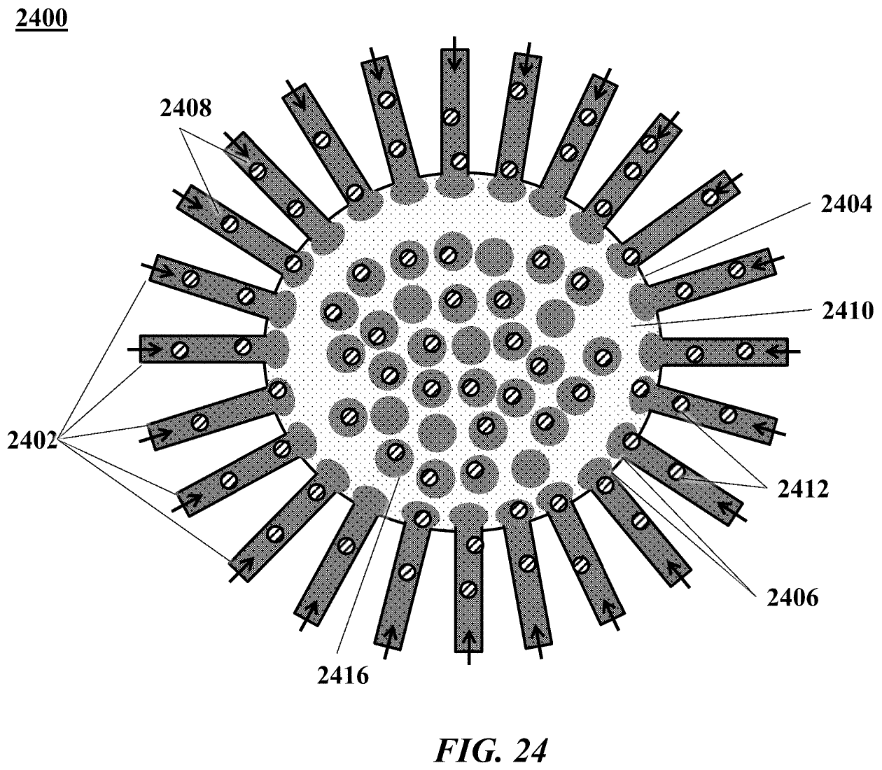

FIG. 24 shows another example of a microfluidic channel structure for increased droplet generation throughput. A microfluidic channel structure 2400 can comprise a plurality of channel segments 2402 arranged generally circularly around the perimeter of a reservoir 2404. Each of the plurality of channel segments 2402 may be in fluid communication with the reservoir 2404. The channel structure 2400 can comprise a plurality of channel junctions 2406 between the plurality of channel segments 2402 and the reservoir 2404. Each channel junction can be a point of droplet generation. The channel segment 2202 from the channel structure 2200 in FIG. 22 and any description to the components thereof may correspond to a given channel segment of the plurality of channel segments 2402 in channel structure 2400 and any description to the corresponding components thereof. The reservoir 2204 from the channel structure 2200 and any description to the components thereof may correspond to the reservoir 2404 from the channel structure 2400 and any description to the corresponding components thereof.

Each channel segment of the plurality of channel segments 2402 may comprise an aqueous fluid 2408 that includes suspended beads 2412. The reservoir 2404 may comprise a second fluid 2410 that is immiscible with the aqueous fluid 2408. In some instances, the second fluid 2410 may not be subjected to and/or directed to any flow in or out of the reservoir 2404. For example, the second fluid 2410 may be substantially stationary in the reservoir 2404. In some instances, the second fluid 2410 may be subjected to flow within the reservoir 2404, but not in or out of the reservoir 2404, such as via application of pressure to the reservoir 2404 and/or as affected by the incoming flow of the aqueous fluid 2408 at the junctions. Alternatively, the second fluid 2410 may be subjected and/or directed to flow in or out of the reservoir 2404. For example, the reservoir 2404 can be a channel directing the second fluid 2410 from upstream to downstream, transporting the generated droplets.

In operation, the aqueous fluid 2408 that includes suspended beads 2412 may be transported along the plurality of channel segments 2402 into the plurality of junctions 2406 to meet the second fluid 2410 in the reservoir 2404 to create a plurality of droplets 2416. A droplet may form from each channel segment at each corresponding junction with the reservoir 2404. At the junction where the aqueous fluid 2408 and the second fluid 2410 meet, droplets can form based on factors such as the hydrodynamic forces at the junction, flow rates of the two fluids 2408, 2410, fluid properties, and certain geometric parameters (e.g., widths and heights of the channel segments 2402, expansion angle of the reservoir 2404, etc.) of the channel structure 2400, as described elsewhere herein. A plurality of droplets can be collected in the reservoir 2404 by continuously injecting the aqueous fluid 2408 from the plurality of channel segments 2402 through the plurality of junctions 2406. Throughput may significantly increase with the substantially parallel channel configuration of the channel structure 2400. A channel structure may have as many substantially parallel channel segments as is practical and allowed for by the size of the reservoir. For example, the channel structure may have at least about 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, 1500, 5000 or more parallel or substantially parallel channel segments. The plurality of channel segments may be substantially evenly spaced apart, for example, around an edge or perimeter of the reservoir. Alternatively, the spacing of the plurality of channel segments may be uneven.

The reservoir 2404 may have an expansion angle, α (not shown in FIG. 24) at or near each channel junction. Each channel segment of the plurality of channel segments 2402 may have a width, w, and a height, h₀, at or near the channel junction. The geometric parameters, w, hₒ, and α, may or may not be uniform for each of the channel segments in the plurality of channel segments 2402. For example, each channel segment may have the same or different widths at or near its respective channel junction with the reservoir 2404. For example, each channel segment may have the same or different height at or near its respective channel junction with the reservoir 2404.

The reservoir 2404 may have the same or different expansion angle at the different channel junctions with the plurality of channel segments 2402. For example, a circular reservoir (as shown in FIG. 24) may have a conical, dome-like, or hemispherical ceiling (e.g., top wall) to provide the same or substantially same expansion angle for each channel segments 2402 at or near the plurality of channel junctions 2406. When the geometric parameters are uniform, beneficially, resulting droplet size may be controlled to be uniform even with the increased throughput. In some instances, when it is desirable to have a different distribution of droplet sizes, the geometric parameters for the plurality of channel segments 2402 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size. The beads and/or biological particle injected into the droplets may or may not have uniform size.

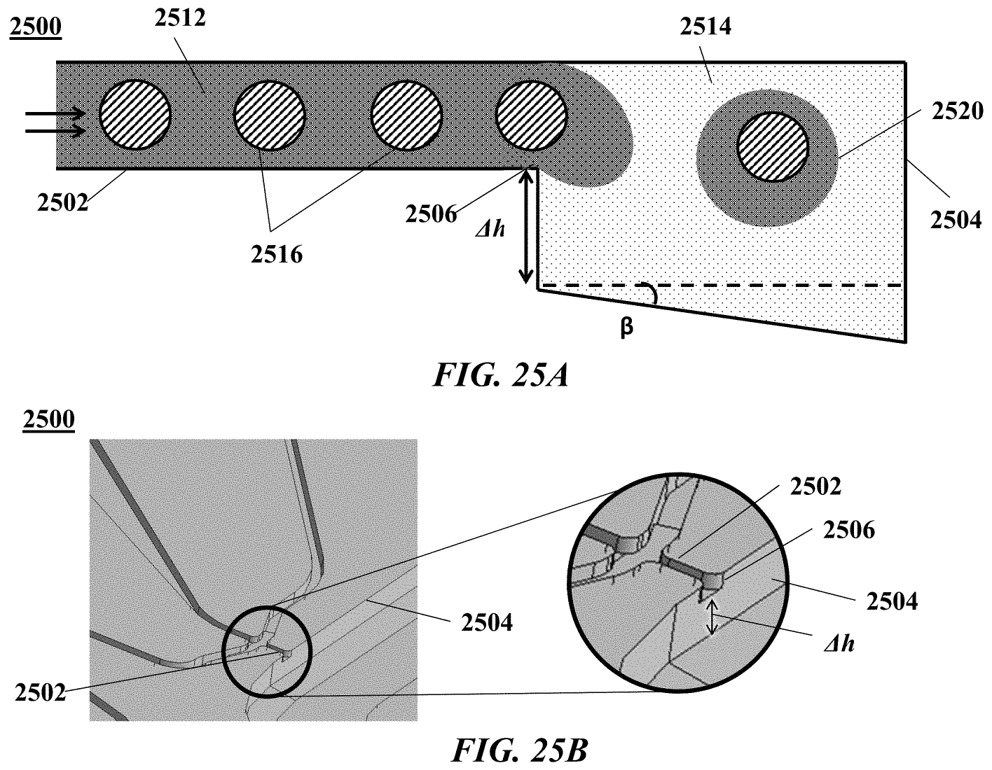

FIG. 25A shows a cross-section view of another example of a microfluidic channel structure with a geometric feature for controlled partitioning. A channel structure 2500 can include a channel segment 2502 communicating at a channel junction 2506 (or intersection) with a reservoir 2504. In some instances, the channel structure 2500 and one or more of its components can correspond to the channel structure 1900 and one or more of its components. FIG. 25B shows a perspective view of the channel structure 2500 of FIG. 25A.

An aqueous fluid 2512 comprising a plurality of particles 2516 may be transported along the channel segment 2502 into the junction 2506 to meet a second fluid 2514 (e.g., oil, etc.) that is immiscible with the aqueous fluid 2512 in the reservoir 2504 to create droplets 2520 of the aqueous fluid 2512 flowing into the reservoir 2504. At the junction 2506 where the aqueous fluid 2512 and the second fluid 2514 meet, droplets can form based on factors such as the hydrodynamic forces at the junction 2506, relative flow rates of the two fluids 2512, 2514, fluid properties, and certain geometric parameters (e.g., Δh, etc.) of the channel structure 2500. A plurality of droplets can be collected in the reservoir 2504 by continuously injecting the aqueous fluid 2512 from the channel segment 2502 at the junction 2506.

A discrete droplet generated may comprise one or more particles of the plurality of particles 2516. As described elsewhere herein, a particle may be any particle, such as a bead, cell bead, gel bead, biological particle, macromolecular constituents of biological particle, or other particles. Alternatively, a discrete droplet generated may not include any particles.

In some instances, the aqueous fluid 2512 can have a substantially uniform concentration or frequency of particles 2516. As described elsewhere herein (e.g., with reference to FIG. 22), the particles 2516 (e.g., beads) can be introduced into the channel segment 2502 from a separate channel (not shown in FIGS. 25A-25B). The frequency of particles 2516 in the channel segment 2502 may be controlled by controlling the frequency in which the particles 2516 are introduced into the channel segment 2502 and/or the relative flow rates of the fluids in the channel segment 2502 and the separate channel. In some instances, the particles 2516 can be introduced into the channel segment 2502 from a plurality of different channels, and the frequency controlled accordingly. In some instances, different particles may be introduced via separate channels. For example, a first separate channel can introduce beads and a second separate channel can introduce biological particles into the channel segment 2502. The first separate channel introducing the beads may be upstream or downstream of the second separate channel introducing the biological particles.

In some instances, the second fluid 2514 may not be subjected to and/or directed to any flow in or out of the reservoir 2504. For example, the second fluid 2514 may be substantially stationary in the reservoir 2504. In some instances, the second fluid 2514 may be subjected to flow within the reservoir 2504, but not in or out of the reservoir 2504, such as via application of pressure to the reservoir 2504 and/or as affected by the incoming flow of the aqueous fluid 2512 at the junction 2506. Alternatively, the second fluid 2514 may be subjected and/or directed to flow in or out of the reservoir 2504. For example, the reservoir 2504 can be a channel directing the second fluid 2514 from upstream to downstream, transporting the generated droplets.

The channel structure 2500 at or near the junction 2506 may have certain geometric features that at least partly determine the sizes and/or shapes of the droplets formed by the channel structure 2500. The channel segment 2502 can have a first cross-section height, h₁, and the reservoir 2504 can have a second cross-section height, h₂. The first cross-section height, h₁, and the second cross-section height, h₂, may be different, such that at the junction 2506, there is a height difference of Δh. The second cross-section height, h₂, may be greater than the first cross-section height, h₁. In some instances, the reservoir may thereafter gradually increase in cross-section height, for example, the more distant it is from the junction 2506. In some instances, the cross-section height of the reservoir may increase in accordance with expansion angle, β, at or near the junction 2506. The height difference, Δh, and/or expansion angle, β, can allow the tongue (portion of the aqueous fluid 2512 leaving channel segment 2502 at junction 2506 and entering the reservoir 2504 before droplet formation) to increase in depth and facilitate decrease in curvature of the intermediately formed droplet. For example, droplet size may decrease with increasing height difference and/or increasing expansion angle.

The height difference, Δh, can be at least about 1 μm. Alternatively, the height difference can be at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 μm or more. Alternatively, the height difference can be at most about 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1 μm or less. In some instances, the expansion angle, β, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less.

In some instances, the flow rate of the aqueous fluid 2512 entering the junction 2506 can be between about 0.04 microliters (μL)/minute (min) and about 40 μL/min. In some instances, the flow rate of the aqueous fluid 2512 entering the junction 2506 can be between about 0.01 microliters (μL)/minute (min) and about 100 μL/min. Alternatively, the flow rate of the aqueous fluid 2512 entering the junction 2506 can be less than about 0.01 μL/min. Alternatively, the flow rate of the aqueous fluid 2512 entering the junction 2506 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min , 120 μL/min , 130 μL/min , 140 μL/min , 150 μL/min, or greater. At lower flow rates, such as flow rates of about less than or equal to 10 microliters/minute, the droplet radius may not be dependent on the flow rate of the aqueous fluid 2512 entering the junction 2506. The second fluid 2514 may be stationary, or substantially stationary, in the reservoir 2504. Alternatively, the second fluid 2514 may be flowing, such as at the above flow rates described for the aqueous fluid 2512.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

While FIGS. 25A and 25B illustrate the height difference, zlh, being abrupt at the junction 2506 (e.g., a step increase), the height difference may increase gradually (e.g., from about 0 μm to a maximum height difference). Alternatively, the height difference may decrease gradually (e.g., taper) from a maximum height difference. A gradual increase or decrease in height difference, as used herein, may refer to a continuous incremental increase or decrease in height difference, wherein an angle between any one differential segment of a height profile and an immediately adjacent differential segment of the height profile is greater than 90°. For example, at the junction 2506, a bottom wall of the channel and a bottom wall of the reservoir can meet at an angle greater than 90°. Alternatively or in addition, a top wall (e.g., ceiling) of the channel and a top wall (e.g., ceiling) of the reservoir can meet an angle greater than 90°. A gradual increase or decrease may be linear or non-linear (e.g., exponential, sinusoidal, etc.). Alternatively or in addition, the height difference may variably increase and/or decrease linearly or non-linearly. While FIGS. 25A and 25B illustrate the expanding reservoir cross-section height as linear (e.g., constant expansion angle, β), the cross-section height may expand non-linearly. For example, the reservoir may be defined at least partially by a dome-like (e.g., hemispherical) shape having variable expansion angles. The cross-section height may expand in any shape.

The channel networks, e.g., as described above or elsewhere herein, can be fluidly coupled to appropriate fluidic components. For example, the inlet channel segments are fluidly coupled to appropriate sources of the materials they are to deliver to a channel junction. These sources may include any of a variety of different fluidic components, from simple reservoirs defined in or connected to a body structure of a microfluidic device, to fluid conduits that deliver fluids from off-device sources, manifolds, fluid flow units (e.g., actuators, pumps, compressors) or the like. Likewise, the outlet channel segment (e.g., channel segment 2008, reservoir 2404, etc.) may be fluidly coupled to a receiving vessel or conduit for the partitioned cells for subsequent processing. Again, this may be a reservoir defined in the body of a microfluidic device, or it may be a fluidic conduit for delivering the partitioned cells to a subsequent process operation, instrument or component.

The methods and systems described herein may be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-dT sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

### Computer Systems

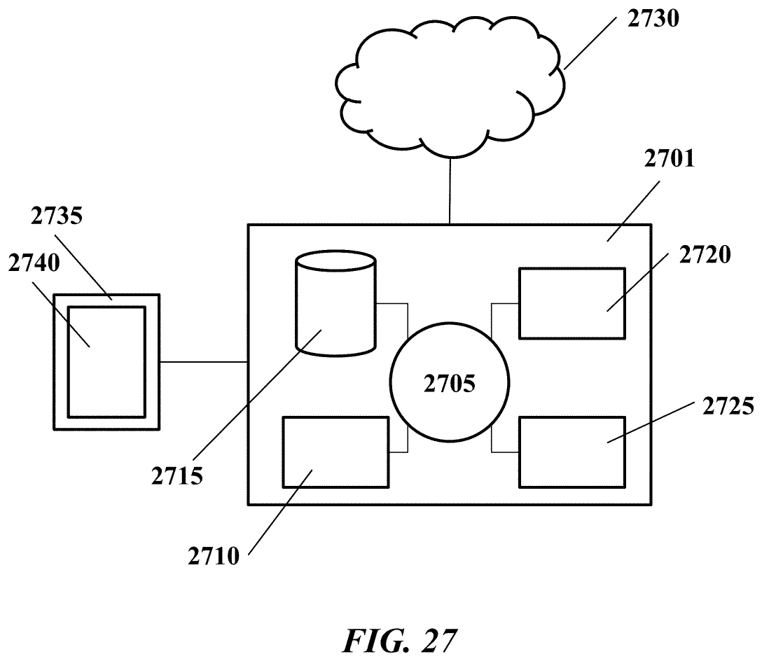

The present disclosure provides computer systems that are programmed to implement methods of the disclosure. FIG. 27 shows a computer system 2701 that is programmed or otherwise configured to for methods of nucleic acid sequencing and determination of genetic variations, storing reference nucleic acid sequences, conducting sequence analysis, and/or comparing sample and reference nucleic acid sequences as described herein. The computer system 2701 can regulate various aspects of the present disclosure, such as, for example, regulating fluid flow rate in one or more channels in a microfluidic structure. The computer system 2701 can be an electronic device of a user or a computer system that is remotely located with respect to the electronic device. The electronic device can be a mobile electronic device.

The computer system 2701 includes a central processing unit (CPU, also “processor” and “computer processor” herein) 2705, which can be a single core or multi core processor, or a plurality of processors for parallel processing. The computer system 2701 also includes memory or memory location 2710 (e.g., random-access memory, read-only memory, flash memory), electronic storage unit 2715 (e.g., hard disk), communication interface 2720 (e.g., network adapter) for communicating with one or more other systems, and peripheral devices 2725, such as cache, other memory, data storage and/or electronic display adapters. The memory 2710, storage unit 2715, interface 2720 and peripheral devices 2725 are in communication with the CPU 2705 through a communication bus (solid lines), such as a motherboard. The storage unit 2715 can be a data storage unit (or data repository) for storing data. The computer system 2701 can be operatively coupled to a computer network (“network”) 2730 with the aid of the communication interface 2720. The network 2730 can be the Internet, an interne and/or extranet, or an intranet and/or extranet that is in communication with the Internet. The network 2730 in some cases is a telecommunication and/or data network. The network 2730 can include one or more computer servers, which can enable distributed computing, such as cloud computing. The network 2730, in some cases with the aid of the computer system 2701, can implement a peer-to-peer network, which may enable devices coupled to the computer system 2701 to behave as a client or a server.

The CPU 2705 can execute a sequence of machine-readable instructions, which can be embodied in a program or software. The instructions may be stored in a memory location, such as the memory 2710. The instructions can be directed to the CPU 2705, which can subsequently program or otherwise configure the CPU 2705 to implement methods of the present disclosure. Examples of operations performed by the CPU 2705 can include fetch, decode, execute, and writeback.

The CPU 2705 can be part of a circuit, such as an integrated circuit. One or more other components of the system 2701 can be included in the circuit. In some cases, the circuit is an application specific integrated circuit (ASIC).

The storage unit 2715 can store files, such as drivers, libraries and saved programs. The storage unit 2715 can store user data, e.g., user preferences and user programs. The computer system 2701 in some cases can include one or more additional data storage units that are external to the computer system 2701, such as located on a remote server that is in communication with the computer system 2701 through an intranet or the Internet.

The computer system 2701 can communicate with one or more remote computer systems through the network 2730. For instance, the computer system 2701 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2701 via the network 2730.

Methods as described herein can be implemented by way of machine (e.g., computer processor) executable code stored on an electronic storage location of the computer system 2701, such as, for example, on the memory 2710 or electronic storage unit 2715. The machine executable or machine readable code can be provided in the form of software. During use, the code can be executed by the processor 2705. In some cases, the code can be retrieved from the storage unit 2715 and stored on the memory 2710 for ready access by the processor 2705. In some situations, the electronic storage unit 2715 can be precluded, and machine-executable instructions are stored on memory 2710.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 2701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2701 can include or be in communication with an electronic display 2735 that comprises a user interface (UI) 2740 for providing, for example, an output or readout of a nucleic acid sequencing instrument coupled to the computer system 2701. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 2705. The algorithm can, for example, perform sequencing, sequence comparison, filtering of sequencing reads for quality control, haplotype determination, RHDO, or SPRT analysis.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for nucleic acid analysis, comprising:
(a) generating a plurality of barcoded parental nucleic acid molecules in a plurality of partitions using (i) a plurality of parental nucleic acid molecules derived from a parental biological sample, and (ii) a plurality of nucleic acid barcode molecules;
(b) enriching said plurality of barcoded parental nucleic acid molecules or derivatives thereof for target nucleic acid molecules comprising one or more target regions to generate an enriched set of barcoded parental nucleic acid molecules;
(c) using said enriched set of barcoded parental nucleic acid molecules or derivatives thereof to generate parental nucleic acid sequence information comprising one or more nucleic acid sequences of said plurality of parental nucleic acid molecules;
(d) processing said parental nucleic acid sequence information to identify one or more maternal or paternal haplotype blocks from said parental biological sample; and
(e) processing cell-free nucleic acid sequence information derived from a maternal cell-free biological sample against said one or more maternal or paternal haplotype blocks, to identify one or more genomic variations in one or more fetal nucleic acid sequences of said maternal cell-free biological sample.

2. The method of claim 1, wherein said processing in (e) comprises performing a relative haplotype dosing analysis.

3. The method of claim 2, wherein performing said relative haplotype dosing analysis comprises performing a sequential probability ratio test of allelic imbalance in said cell-free nucleic acid sequence information derived from a maternal cell-free biological sample.

4. The method of claim 1, further comprising, prior to (a), generating a plurality of partitions comprising (i) said plurality of parental nucleic acid molecules, and (ii) said plurality of nucleic acid barcode molecules.

5. The method of claim 1, wherein in (c), said parental nucleic acid sequence information is generated by sequencing said enriched set of barcoded parental nucleic acid molecules or derivatives thereof.

6. The method of claim 1, wherein prior to (b), said plurality of barcoded parental nucleic acid molecules are removed or released from said plurality of partitions.

7. The method of claim 6, wherein said enriching of (b) is performed using nucleic acid capture of said one or more target regions in said plurality of barcoded parental nucleic acid molecules.

8. The method of claim 7, wherein said nucleic acid capture is exome capture.

9. The method of claim 1, wherein said enriching of (b) is performed by nucleic acid amplification of said one or more target regions in said plurality of barcoded parental nucleic acid molecules.

10. The method of claim 1, further comprising obtaining, from a subject having a fetus, a maternal biological sample, and deriving from said maternal biological sample (i) said plurality of parental nucleic acid molecules, and (ii) said maternal cell-free biological sample comprising one or more fetal nucleic acid molecules of said fetus.

11. The method of claim 10, further comprising sequencing said one or more fetal nucleic acid molecules of said maternal cell-free biological sample to generate said cell-free nucleic acid sequence information.

12. The method of claim 1, wherein in (a), said plurality of parental nucleic acid molecules is derived from a maternal biological sample, and wherein said parental nucleic acid sequence information in (d) comprises one or more haplotype blocks derived from said maternal biological sample.

13. The method of claim 12, further comprising generating paternal nucleic acid sequence information from a plurality of nucleic acid molecules derived from a paternal biological sample, and processing said paternal nucleic acid sequence information to identify one or more maternal or paternal haplotype blocks from said paternal biological sample.

14. The method of claim 1, wherein a given partition of said plurality of partitions comprises a parental nucleic acid molecule from said plurality of parental nucleic acid molecules, wherein said parental nucleic acid molecule has a length longer than 10 kilobases.

15. The method of claim 14, wherein said parental nucleic acid molecule has a length longer than 100 kilobases.

16. The method of claim 1, wherein said plurality of partitions further comprise a plurality of beads, wherein a given bead of said plurality of beads comprises a plurality of nucleic acid barcode molecules attached thereto, and wherein a given partition of said plurality of partitions further comprises a single bead.

17. The method of claim 16, wherein said plurality of partitions is a plurality of droplets or a plurality of wells.

18. A method for nucleic acid analysis, comprising:
(a) providing a plurality of parental nucleic acid molecules derived from a parental biological sample and a plurality of beads, wherein a given bead of said plurality of beads comprises a plurality of nucleic acid barcode molecules attached thereto, and wherein said plurality of nucleic acid barcode molecules comprise a sequence complementary to one or more target sequences of said plurality of parental nucleic acid molecules;
(b) generating a plurality of partitions, wherein a given partition of said plurality of partitions comprises (i) a parental nucleic acid molecule from said plurality of parental nucleic acid molecules, and (ii) a single bead from said plurality of beads;
(c) in said plurality of partitions, synthesizing a plurality of barcoded, targeted parental nucleic acid molecules using (i) parental nucleic acid molecules from said plurality of parental nucleic acid molecules, and (ii) nucleic acid barcode molecules from said plurality of nucleic acid barcode molecules, wherein said barcoded, targeted parental nucleic acid molecules comprise said one or more target sequences;
(d) using said barcoded, targeted parental nucleic acid molecules or derivatives thereof to generate parental nucleic acid sequence information comprising one or more nucleic acid sequences of said plurality of parental nucleic acid molecules;
(e) processing said parental nucleic acid sequence information to identify one or more maternal or paternal haplotype blocks from said parental biological sample; and
(f) processing cell-free nucleic acid sequence information derived from a maternal cell-free biological sample against said one or more maternal or paternal haplotype blocks, to identify one or more genomic variations in one or more fetal nucleic acid sequences of said cell-free nucleic acid sequence information.

19. The method of claim 18, wherein said processing in (f) comprises performing a relative haplotype dosing analysis.

20. The method of claim 19, wherein performing said relative haplotype dosing analysis comprises performing a sequential probability ratio test of allelic imbalance in said cell-free nucleic acid sequence information derived from a maternal cell-free biological sample.

21. The method of claim 18, wherein in (d), said parental nucleic acid sequence information is generated by sequencing said barcoded, targeted parental nucleic acid molecules or derivatives thereof.

22. The method of claim 18, further comprising obtaining, from a subject having a fetus, a maternal biological sample, and deriving from said maternal biological sample (i) said plurality of parental nucleic acid molecules, and (ii) said maternal cell-free biological sample comprising one or more fetal nucleic acid molecules of said fetus.

23. The method of claim 22, further comprising sequencing said one or more fetal nucleic acid molecules of said maternal cell-free biological sample to generate said cell-free nucleic acid sequence information.

24. The method of claim 18, wherein in (a), said plurality of parental nucleic acid molecules is derived from a maternal biological sample, and wherein said parental nucleic acid sequence information in (e) comprises one or more haplotype blocks derived from said maternal biological sample.

25. The method of claim 24, further comprising generating paternal nucleic acid sequence information from a plurality of nucleic acid molecules derived from a paternal biological sample, and processing said paternal nucleic acid sequence information to identify one or more maternal or paternal haplotype blocks from said parental biological sample.

26. The method of claim 18, wherein said parental nucleic acid molecule from said plurality of parental nucleic acid molecules has a length longer than 1 kilobase (kb).

27. The method of claim 26, wherein said parental nucleic acid molecule from said plurality of parental nucleic acid molecules has a length longer than 10 kb.

28. The method of claim 18, wherein said plurality of partitions is a plurality of droplets or a plurality of wells.

29. A method for nucleic acid analysis, comprising:
(a) generating a plurality of partitions comprising (i) a plurality of parental nucleic acid molecules derived from a parental biological sample, (ii) a plurality of nucleic acid barcode molecules, and (iii) a plurality of oligonucleotide primers, wherein said plurality of oligonucleotide primers is capable of amplifying one or more target sequences of said plurality of parental nucleic acid molecules;
(b) in said plurality of partitions, generating a plurality of amplified parental nucleic acid molecules using (i) nucleic acid molecules from said plurality of parental nucleic acid molecules, and (ii) oligonucleotide primers from said plurality of oligonucleotide primers;
(c) in said plurality of partitions, generating a plurality of barcoded, amplified parental nucleic acid molecules using (i) amplified parental nucleic acid molecules from said plurality of amplified parental nucleic acid molecules and (ii) nucleic acid barcode molecules from said plurality of nucleic acid barcode molecules;
(d) sequencing said plurality of barcoded, amplified parental nucleic acid molecules or derivatives thereof to generate parental nucleic acid sequence information comprising one or more nucleic acid sequences of said plurality of parental nucleic acid molecules;
(e) processing said parental nucleic acid sequence information to identify one or more maternal or paternal haplotype blocks from said parental biological sample; and
(f) processing cell-free nucleic acid sequence information derived from a maternal cell-free biological sample against said one or more maternal or paternal haplotype blocks, to identify one or more genomic variations in one or more fetal nucleic acid sequences of said cell-free nucleic acid sequence information.

30. The method of claim 29, wherein said processing in (f) comprises performing a relative haplotype dosing analysis.
